(19) United States
(12) Patent Application Publication
(10) Pub. No.: US 2022/0152037 A1
(43) Pub. Date: May 19, 2022

(54) USE OF GABAA RECEPTOR MODULATORS FOR TREATMENT OF PAIN

(71) Applicant: NEUROCYCLE THERAPEUTIC, INC., Cambridge, MA (US)

(72) Inventors: Matthew TOCZKO, Cambridge, MA (US); Jed Hubbs, Cambridge, MA (US)

(21) Appl. No.: 17/439,151

(22) PCT Filed: Mar. 18, 2020
(86) PCT No.: PCT/US20/23355

§ 371 Date: Sep. 14, 2021

Related U.S. Application Data
(60) Provisional application No. 62/819,794, filed on Mar. 18, 2019.

(51) Int. Cl. A61K 31/53; A61P 25/04; A61K 31/5517

(52) U.S. Cl. CPC A61K 31/53; A61K 2121/00; A61K 31/5517; A61P 25/04

(57) ABSTRACT

Disclosed herein are GABA_{A} receptor modulators and compositions comprising GABA_{A} receptor modulators for treatment of pain and associated conditions such as fibromyalgia. Also disclosed herein are methods of treating pain and associated conditions in a subject by administering a GABA_{A} receptor modulators or composition as described herein.

### CROSS-REFERENCE

This application claims benefit of U.S. Provisional Patent Application No. 62/819,794 filed on Mar. 18, 2019, which is incorporated herein by reference in its entirety.

### SUMMARY

Disclosed herein are methods of treating fibromyalgia comprising administering to a subject in need thereof a therapeutically effective amount of a compound that is a GABA_{A} receptor modulator. Also disclosed herein are compositions for use in treating fibromyalgia comprising a therapeutically effective amount of a compound that is a GABA_{A} receptor modulator. In some embodiments, the compound is a positive GABA_{A} receptor modulator. In some embodiments, the compound is a positive allosteric GABA_{A} receptor modulator. In some embodiments, the compound is an α1, α2, α3, or α5 GABA_{A} receptor modulator. In some embodiments, the compound is a positive allosteric α2 or α3 GABA_{A} receptor modulator. In some embodiments, the compound is of the general formula (1a), general formula (1b), general formula (1c), or a pharmaceutically acceptable salt or solvate thereof,

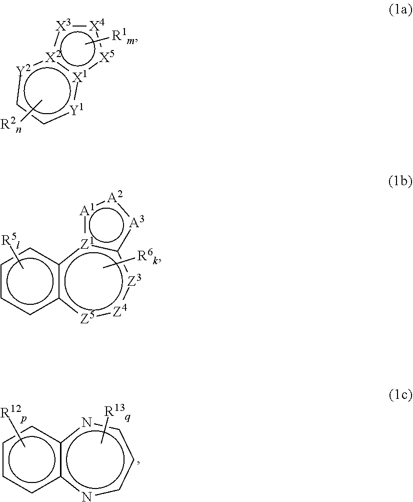

wherein
   X¹, X², X³, X⁴ and X⁵ are each independently —C, —N, —S or —O, wherein at least two of X¹, X², X³, X⁴ and X⁵ are —N,
   Y¹ and Y² are each independently —C or —N,
   m of R¹ₘ is 1, wherein R¹ is an unsubstituted phenyl, a phenyl substituted with C₁-C₄-alkyl, F, Cl, Br, I, —CN, a substituted or unsubstituted biphenyl or —(C═O)—R³, wherein R³ is a substituted or unsubstituted aryl or 5- to 6-membered heteroaryl,
      n of R²ₙ is 1 or 2, wherein each R² is independently a substituted or unsubstituted C₃-C₈ cycloalkyl, a substituted or unsubstituted C₁-C₆ alkyl, a substituted or unsubstituted C₁-C₆ alcohol, a substituted or unsubstituted 6-membered heteroaryl, a halogen, or —O—CH₂—R⁴, wherein R⁴ is a substituted or unsubstituted 5- or 6-membered heteroaryl,
   Z¹, Z³, Z⁴, and Z⁵ are each independently —C, —N, —S or —O,
   A¹, A² and A³, are each independently —C, —N, —(C═O)—O—R⁷, or

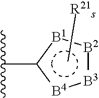

wherein
   R⁷ is alkyl,
   B¹, B², B³, and B⁴ are each independently —C, —N, or —O,
   s of R²¹ₛ is 1, 2, 3 or 4, and
   R²¹ is hydrogen or C₁-C₆ alkyl,
l of R⁵ₗ is 1 or 2, wherein each R⁵ is independently a C₁-C₄ alkinyl or a halogen,
k of R⁶ₖ is 1, 2, 3 or 4, wherein each R⁶ is independently a substituted or unsubstituted aryl, a substituted or unsubstituted heteroaryl, a substituted or unsubstituted C₁-C₃ alkyl, or hydrogen,
p of R¹² is 1 or 2, wherein each R¹² is independently a substituted or unsubstituted C₁-C₄-alkyl, I, Br, Cl or F, and
q of R¹³ is 1, 2, 3 or 4, wherein each R¹³ is independently a substituted or unsubstituted aryl, a substituted or unsubstituted heteroaryl, a substituted or unsubstituted C₁-C₃ alkyl, oxygen or hydrogen.
   In some embodiments, the compound is of the general formula (2), (3), (4), (5), (1c), (7), or a pharmaceutically acceptable salt or solvate thereof,

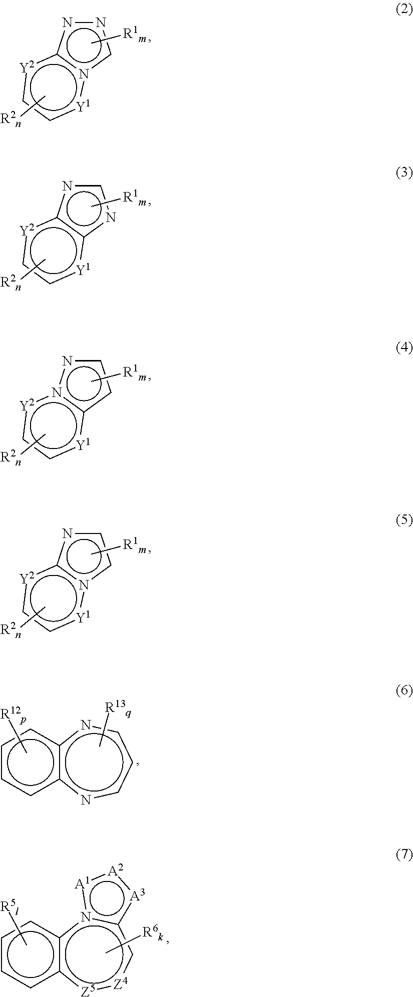

In some embodiments, the compound is of the general formula (2a), (3a), (4a), (5a), (5b), (1c), (7a), or a pharmaceutically acceptable salt or solvate thereof,

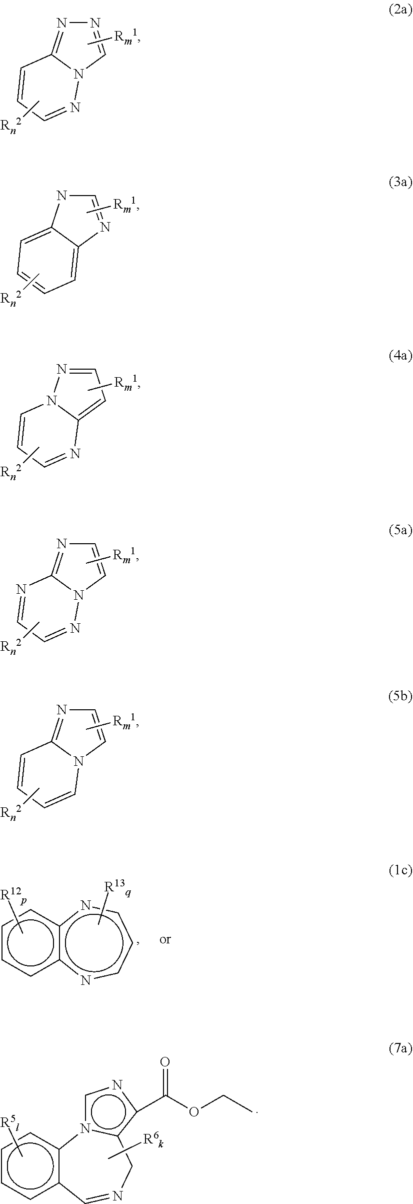

In some embodiments, m of R¹ is 1, and wherein R¹ is: an unsubstituted phenyl, a substituted phenyl comprising C₁-C₄-alkyl, F, Cl, Br, I, —CN as substituents, an unsubstituted biphenyl, a substituted biphenyl comprising at least one —CN as a substituent, a substituted biphenyl comprising at least one —CN as a substituent, or —(C═O)—R³, wherein R³ is pyridine. In some embodiments, the compound is of the general formula (2a′), (3a′), (4a′), (5a′), (5b′), (VI), (7a′), or a pharmaceutically acceptable salt or solvate thereof,

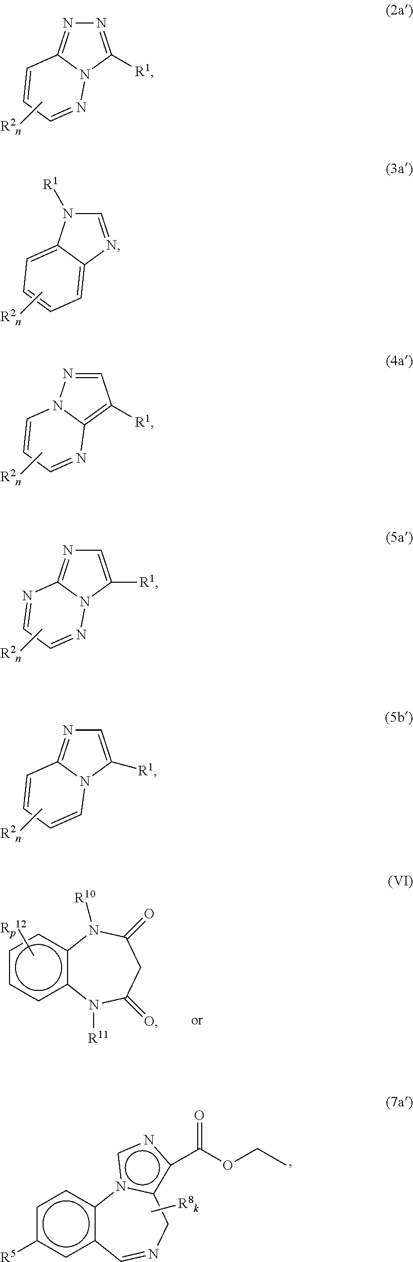

wherein
   R₁₀ is a substituted or unsubstituted aryl, a substituted or unsubstituted C₁-C₃ alkyl or hydrogen,
   R₁₁ is a substituted or unsubstituted aryl, a substituted or unsubstituted C₁-C₃ alkyl, or hydrogen, or
   p of R¹² is 1 and R¹² is I, Br, Cl or F.
      In some embodiments, the compound is of the general formula (2a″), (3a″), (4a″), (5a″), (5b″), (VIa″), (7a″), or a pharmaceutically acceptable salt or solvate thereof,

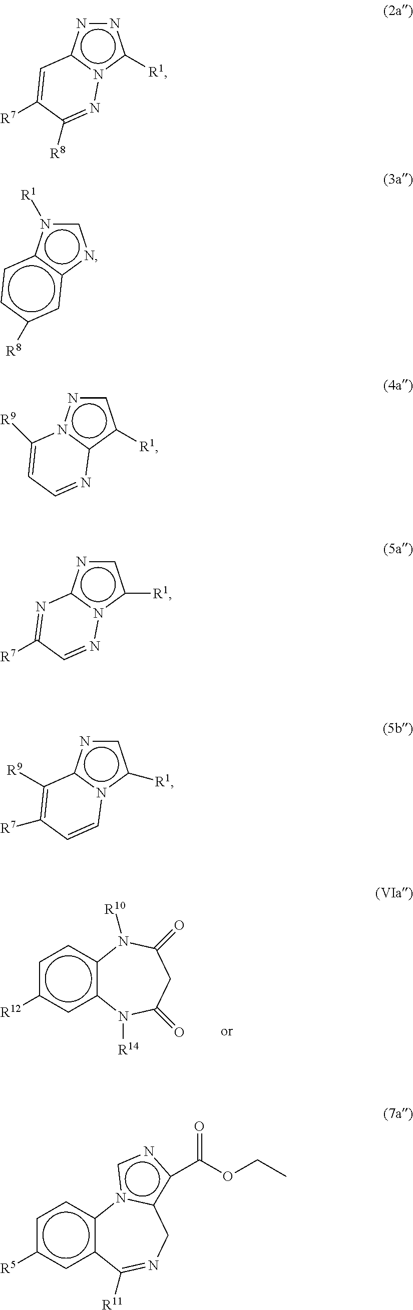

wherein R⁷ is:
   an unsubstituted C₁-C₆ alkyl,
   an unsubstituted C₃-C₈ cycloalkyl,
   an unsubstituted C₁-C₆ alcohol,
R⁸ is:
   —O—CH₂—R⁴, wherein R⁴ is a substituted or unsubstituted 5-membered heteroaryl, or
   an unsubstituted C₁-C₆ alcohol,
R⁹ is:
   an unsubstituted C₆ heteroaryl, or
   a halogen, or
   R⁹ is an unsubstituted 6-membered heteroaryl in formula (4a″) or a halogen in formula (5b″),
R¹⁰ is a C₁-C₃ alkyl or hydrogen,
R¹¹ is a substituted or unsubstituted aryl or heteroaryl,
R¹⁴ is a substituted or unsubstituted aryl or heteroaryl,
R¹² is I, Cl, Br or F, or
R⁵ is C₂ alkinyl or I.
   In some embodiments, the compound is of the general formula (8), or a pharmaceutically acceptable salt or solvate thereof:

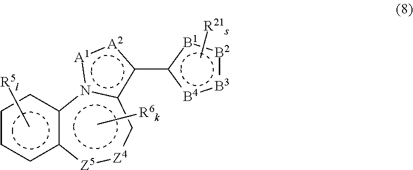

wherein
   Z⁴ and Z⁵ are independently r —C, —N, —S or —O,
   A¹ and A² are independently —C, —N or —(C═O)—O—R⁷, wherein R⁷ is alkyl,
   B¹, B², B³, and B⁴ are independently —C, —N, or —O,
   l of R⁵ₗ is 1 or 2, wherein R⁵ is C₁-C₄ alkinyl or a halogen,
   k of R⁶ₖ is 1, 2, 3 or 4, wherein each R⁶ is independently a substituted or unsubstituted aryl, a substituted or unsubstituted heteroaryl, a substituted or unsubstituted C₁-C₃ alkyl, or hydrogen, and
   s of R²¹ₛ is 1, 2, 3 or 4, wherein each R²¹ is independently hydrogen or C₁-C₆ alkyl.
      In some embodiments, the compound is of the general formula (5a″), or a pharmaceutically acceptable salt or solvate thereof:

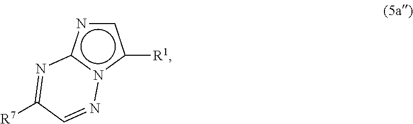

In some embodiments, R¹ is: an unsubstituted biphenyl, a substituted biphenyl comprising at least one —CN as a substituent, or a substituted biphenyl comprising at least one F as a substituent. In some embodiments, R¹ is a substituted biphenyl comprising —CN and F. In some embodiments, R⁷ is an unsubstituted C₁-C₆ alcohol. Disclosed herein are methods of treating fibromyalgia comprising administering to a subject in need thereof a therapeutically effective amount of a compound that is a GABA_{A} receptor modulator, wherein the compound is

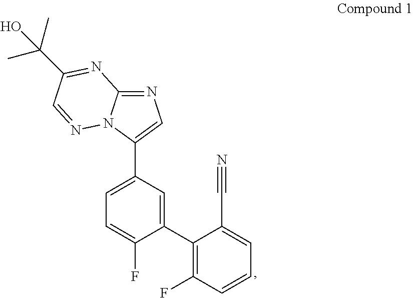 [SMILES: CC(C)(O)c1cnn2c(-c3ccc(F)c(-c4c(F)cccc4C#N)c3)cnc2n1]

or a pharmaceutically acceptable salt or solvent thereof. In some embodiments, the subject is a human. In some embodiments, the subject is a dog.

Disclosed herein are methods of treating pain in a subject comprising administering to the subject an amount of a pharmaceutical composition comprising a compound selected from the group consisting of:

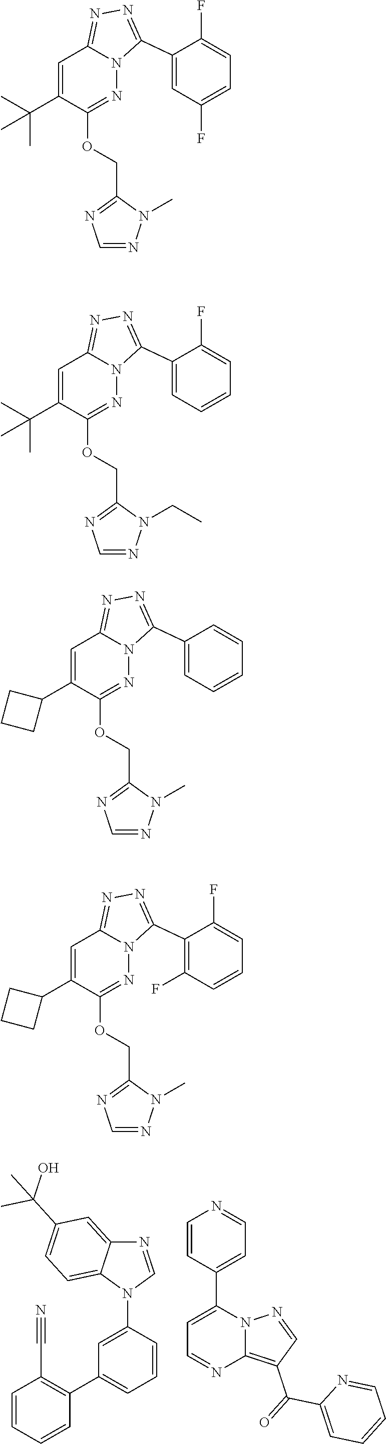

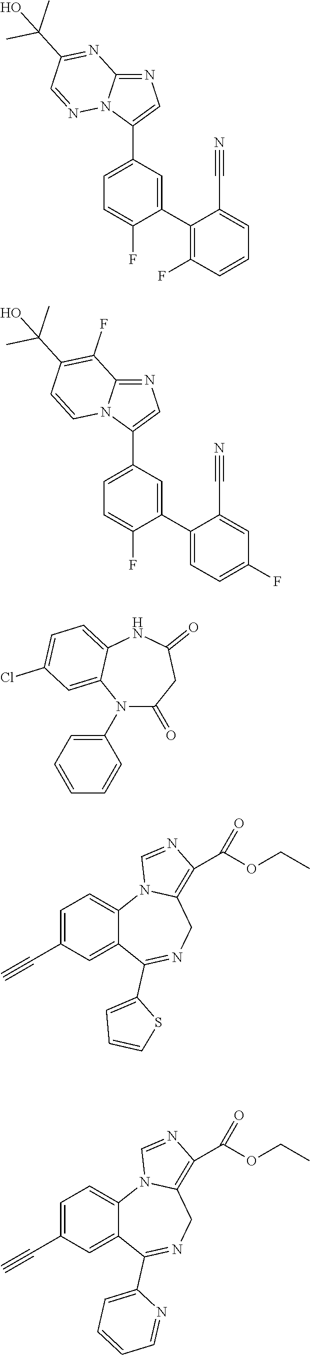

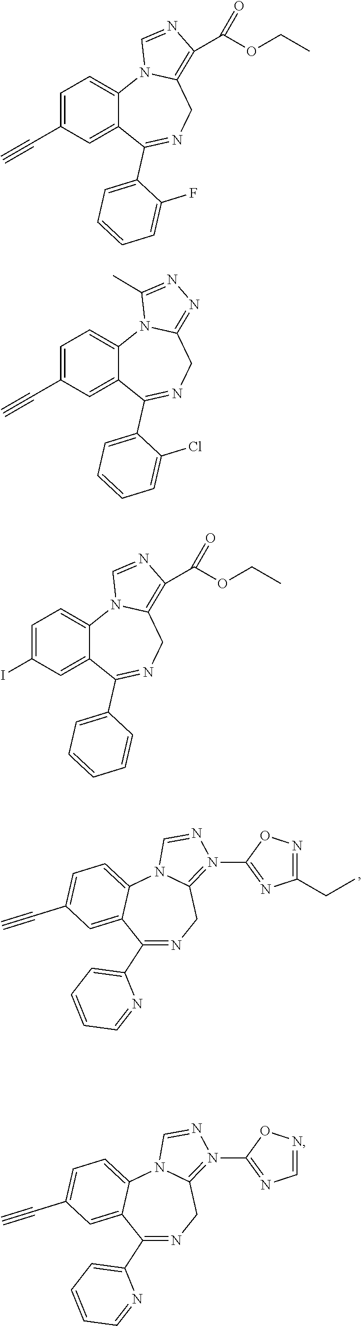

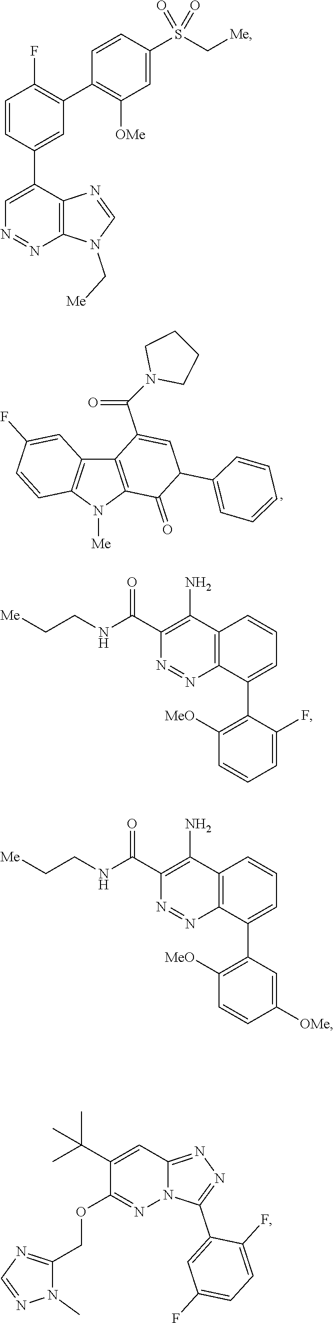 [SMILES: CCn1cnc2c(-c3ccc(F)c(-c4ccc(S(=O)(=O)CC)cc4OC)c3)cnnc21 ; Cn1c2c(c3cc(F)ccc31)C(C(=O)N1CCCC1)=CC(c1ccccc1)C2=O ; CCCNC(=O)c1nnc2c(-c3c(F)cccc3OC)cccc2c1N ; CCCNC(=O)c1nnc2c(-c3cc(OC)ccc3OC)cccc2c1N ; Cn1ncnc1COc1nn2c(-c3cc(F)ccc3F)nnc2cc1C(C)(C)C]

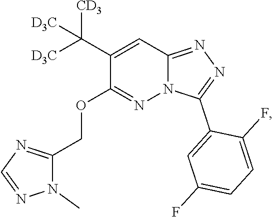 [SMILES: [2H]C([2H])([2H])C(c1cc2nnc(-c3cc(F)ccc3F)n2nc1OCc1ncnn1C)(C([2H])([2H])[2H])C([2H])([2H])[2H]]

and pharmaceutically acceptable salts or solvents thereof, wherein the amount is effective to treat the pain when the compound, or a pharmaceutically acceptable salt or solvent thereof, is administered at a dose of from about 0.001 mg/kg per day to about 3.0 mg/kg per day of a body weight of the subject. Also disclosed herein are compositions for use in treating pain comprising a compound selected from the group consisting of:

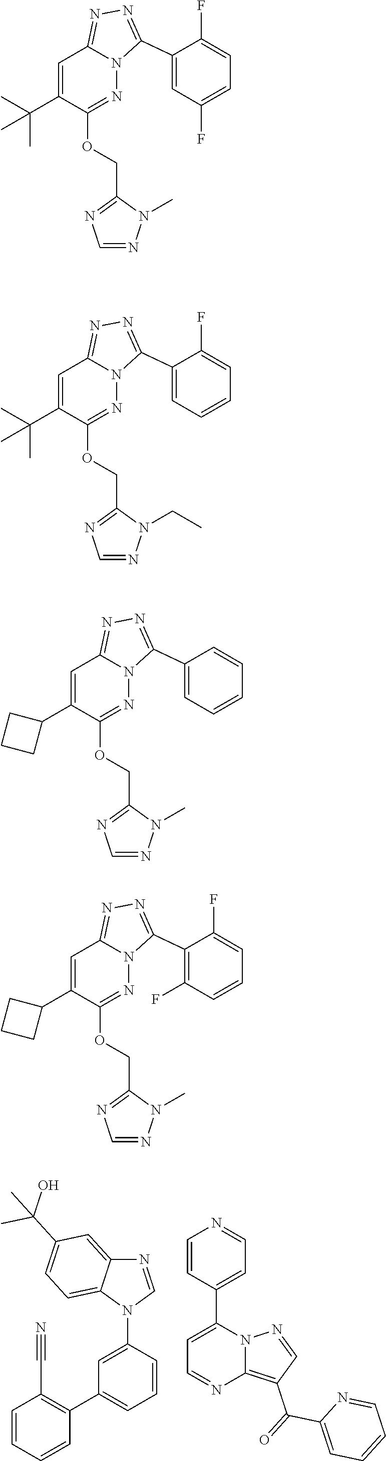

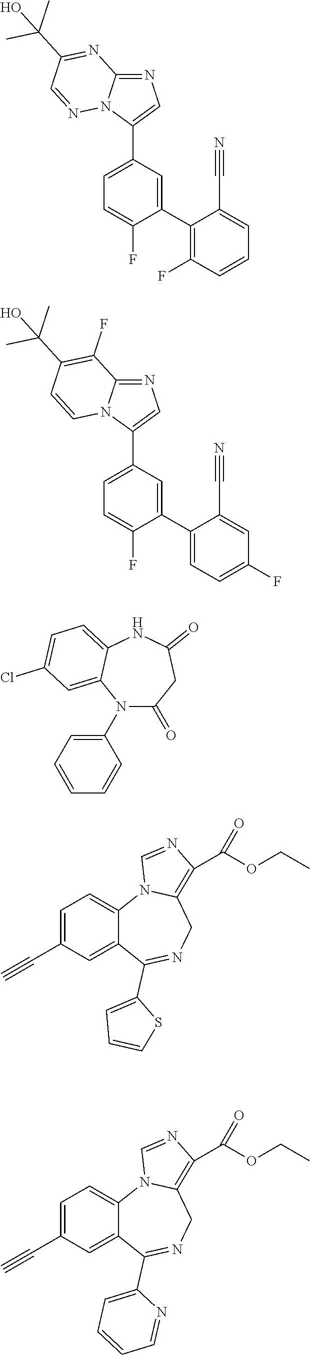

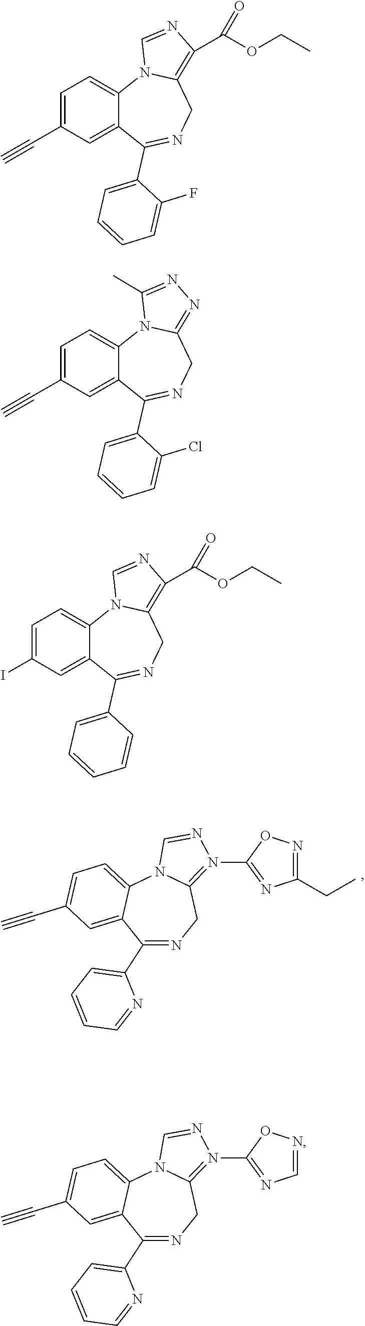

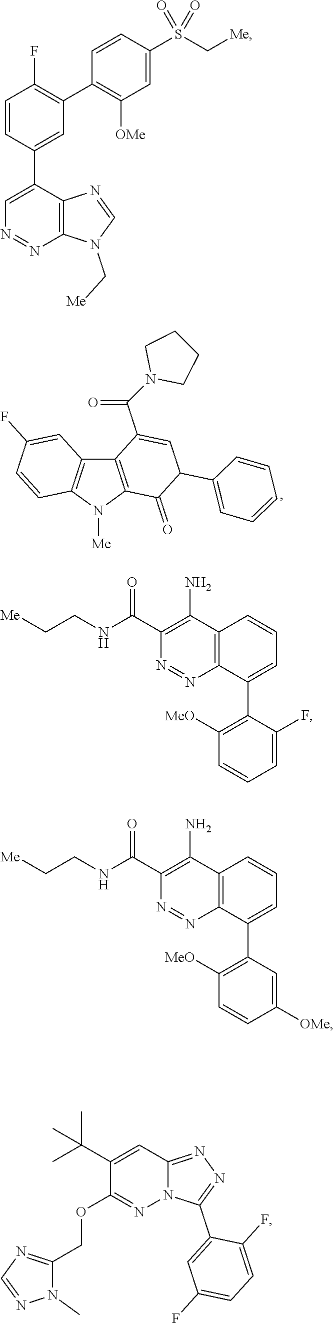 [SMILES: CCn1cnc2c(-c3ccc(F)c(-c4ccc(S(=O)(=O)CC)cc4OC)c3)cnnc21 ; Cn1c2c(c3cc(F)ccc31)C(C(=O)N1CCCC1)=CC(c1ccccc1)C2=O ; CCCNC(=O)c1nnc2c(-c3c(F)cccc3OC)cccc2c1N ; CCCNC(=O)c1nnc2c(-c3cc(OC)ccc3OC)cccc2c1N ; Cn1ncnc1COc1nn2c(-c3cc(F)ccc3F)nnc2cc1C(C)(C)C]

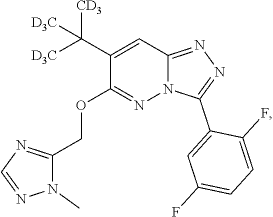 [SMILES: [2H]C([2H])([2H])C(c1cc2nnc(-c3cc(F)ccc3F)n2nc1OCc1ncnn1C)(C([2H])([2H])[2H])C([2H])([2H])[2H]]

and pharmaceutically acceptable salts or solvents thereof, wherein the compound or pharmaceutically acceptable salt or solvent thereof is present in a unit dose sufficient to deliver from about 0.001 mg/kg to about 3.0 mg/kg of the compound with respect to a body weight of a subject. In some embodiments, the compound is:

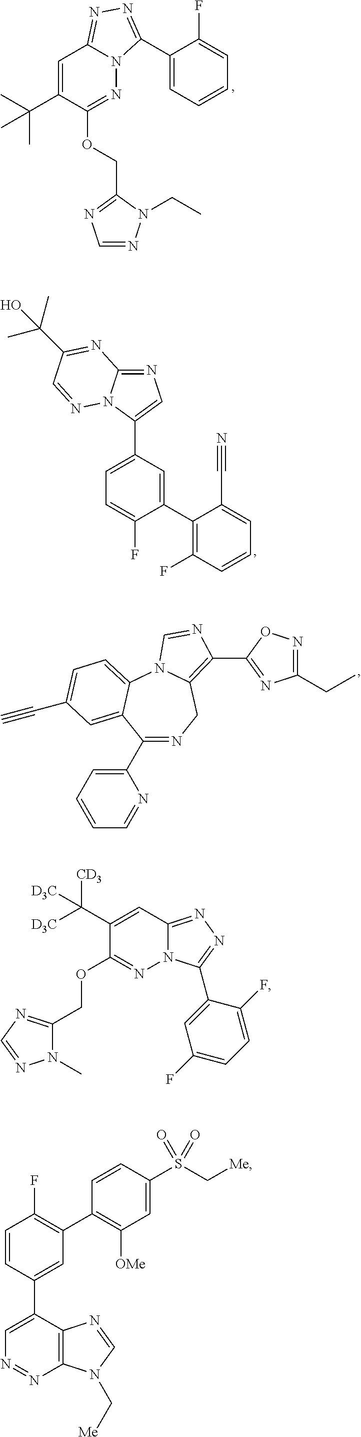 [SMILES: CCn1ncnc1COc1nn2c(-c3ccccc3F)nnc2cc1C(C)(C)C ; CC(C)(O)c1cnn2c(-c3ccc(F)c(-c4c(F)cccc4C#N)c3)cnc2n1 ; C#Cc1ccc2c(c1)C(c1ccccn1)=NCc1c(-c3nc(CC)no3)ncn1-2 ; [2H]C([2H])([2H])C(c1cc2nnc(-c3cc(F)ccc3F)n2nc1OCc1ncnn1C)(C([2H])([2H])[2H])C([2H])([2H])[2H] ; CCn1cnc2c(-c3ccc(F)c(-c4ccc(S(=O)(=O)CC)cc4OC)c3)cnnc21]

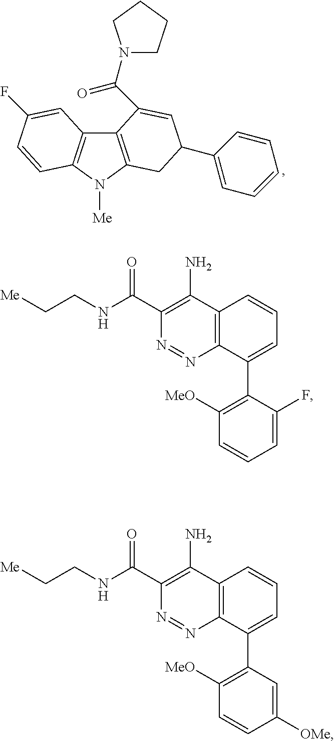 [SMILES: Cn1c2c(c3cc(F)ccc31)C(C(=O)N1CCCC1)=CC(c1ccccc1)C2 ; CCCNC(=O)c1nnc2c(-c3c(F)cccc3OC)cccc2c1N ; CCCNC(=O)c1nnc2c(-c3cc(OC)ccc3OC)cccc2c1N]

or a pharmaceutically acceptable salt or solvent thereof. In some embodiments, the compound is

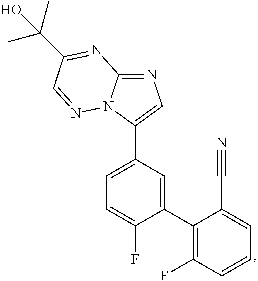 [SMILES: CC(C)(O)c1cnn2c(-c3ccc(F)c(-c4c(F)cccc4C#N)c3)cnc2n1]

or a pharmaceutically acceptable salt or solvent thereof. In some embodiments, the method comprises administering an amount of a compound, or a pharmaceutically acceptable salt or solvent thereof at a dose of at most about 0.3 mg/kg per day of a body weight of the subject. In some embodiments, the method comprises administering an amount of a compound, or a pharmaceutically acceptable salt or solvent thereof at a dose of at most about 0.03 mg/kg per day of a body weight of the subject. In some embodiments, the composition for use comprises a unit dose sufficient to deliver at most about 0.3 mg/kg per day of a body weight of a subject. In some embodiments, the composition for use comprises a unit dose sufficient to deliver at most about 0.03 mg/kg per day of a body weight of the subject. In some embodiments, the pain is related to central nervous system sensitization. In some embodiments, the pain is chronic. In some embodiments, the subject is a human. In some embodiments, the subject is a dog. In some embodiments, the pharmaceutical composition or composition for use further comprises a pharmaceutically acceptable excipient, diluent, or carrier. In some embodiments, the pharmaceutical composition or composition for use comprises a carrier, and wherein the carrier is methyl cellulose. In some embodiments, the administering comprises an oral administration. In some embodiments, the administering is performed at least once a day.

Disclosed herein are methods of treating fibromyalgia in a subject in need thereof comprising administering to the subject a pharmaceutical composition comprising a compound of:

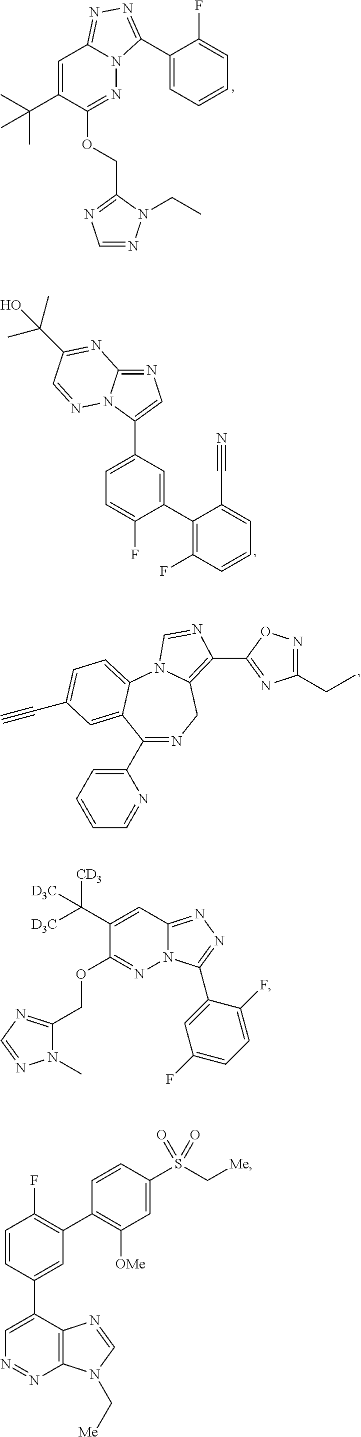 [SMILES: CCn1ncnc1COc1nn2c(-c3ccccc3F)nnc2cc1C(C)(C)C ; CC(C)(O)c1cnn2c(-c3ccc(F)c(-c4c(F)cccc4C#N)c3)cnc2n1 ; C#Cc1ccc2c(c1)C(c1ccccn1)=NCc1c(-c3nc(CC)no3)ncn1-2 ; [2H]C([2H])([2H])C(c1cc2nnc(-c3cc(F)ccc3F)n2nc1OCc1ncnn1C)(C([2H])([2H])[2H])C([2H])([2H])[2H] ; CCn1cnc2c(-c3ccc(F)c(-c4ccc(S(=O)(=O)CC)cc4OC)c3)cnnc21]

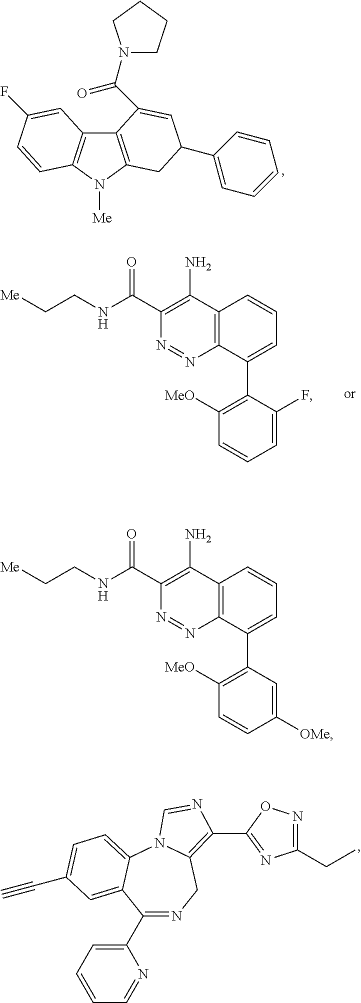 [SMILES: Cn1c2c(c3cc(F)ccc31)C(C(=O)N1CCCC1)=CC(c1ccccc1)C2 ; CCCNC(=O)c1nnc2c(-c3c(F)cccc3OC)cccc2c1N ; "or" ; CCCNC(=O)c1nnc2c(-c3cc(OC)ccc3OC)cccc2c1N ; C#Cc1ccc2c(c1)C(c1ccccn1)=NCc1c(-c3nc(CC)no3)ncn1-2]

or a pharmaceutically acceptable salt or solvent thereof, and a pharmaceutically acceptable excipient, diluent, or carrier. Also disclosed herein are compositions for use in treating fibromyalgia comprising a compound selected from the group consisting of:

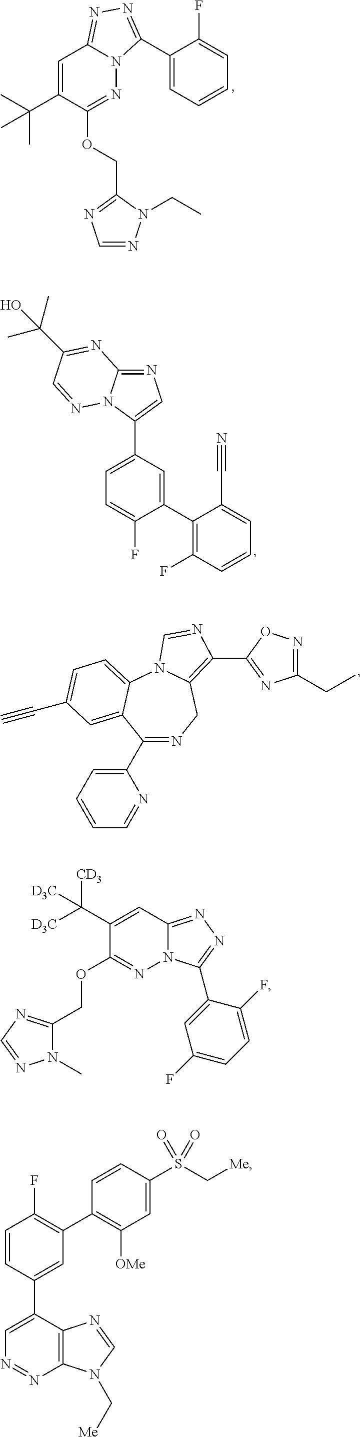 [SMILES: CCn1ncnc1COc1nn2c(-c3ccccc3F)nnc2cc1C(C)(C)C ; CC(C)(O)c1cnn2c(-c3ccc(F)c(-c4c(F)cccc4C#N)c3)cnc2n1 ; C#Cc1ccc2c(c1)C(c1ccccn1)=NCc1c(-c3nc(CC)no3)ncn1-2 ; [2H]C([2H])([2H])C(c1cc2nnc(-c3cc(F)ccc3F)n2nc1OCc1ncnn1C)(C([2H])([2H])[2H])C([2H])([2H])[2H] ; CCn1cnc2c(-c3ccc(F)c(-c4ccc(S(=O)(=O)CC)cc4OC)c3)cnnc21]

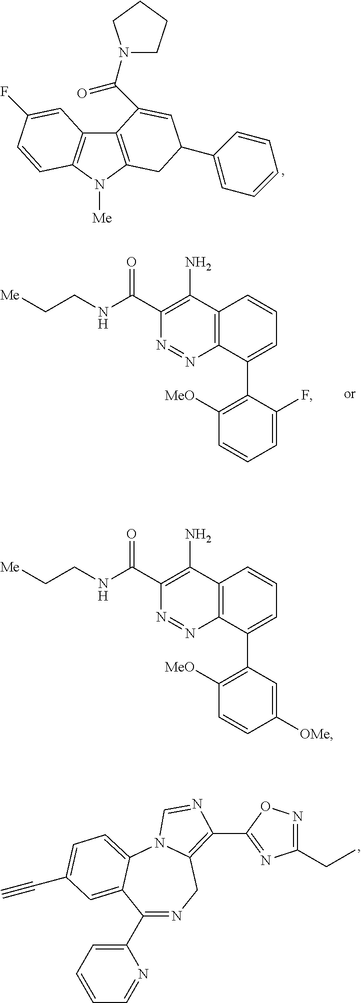 [SMILES: Cn1c2c(c3cc(F)ccc31)C(C(=O)N1CCCC1)=CC(c1ccccc1)C2 ; CCCNC(=O)c1nnc2c(-c3c(F)cccc3OC)cccc2c1N ; "or" ; CCCNC(=O)c1nnc2c(-c3cc(OC)ccc3OC)cccc2c1N ; C#Cc1ccc2c(c1)C(c1ccccn1)=NCc1c(-c3nc(CC)no3)ncn1-2]

or a pharmaceutically acceptable salt or solvent thereof, and a pharmaceutically acceptable excipient, diluent, or carrier. In some embodiments, the pharmaceutical composition or composition for use comprises

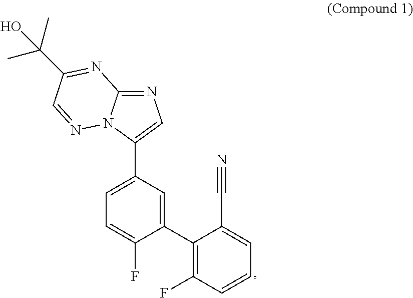 [SMILES: CC(C)(O)c1cnn2c(-c3ccc(F)c(-c4c(F)cccc4C#N)c3)cnc2n1]

or a pharmaceutically acceptable salt or solvent thereof. In some embodiments, the pharmaceutical composition or composition for use comprises

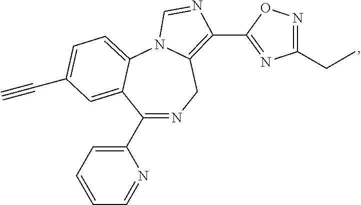 [SMILES: C#Cc1ccc2c(c1)C(c1ccccn1)=NCc1c(-c3nc(CC)no3)ncn1-2]

or a pharmaceutically acceptable salt or solvent thereof. In some embodiments, the method comprises administering the compound or a pharmaceutically acceptable salt or solvent thereof at a dose of from about 0.001 mg/kg per day to about 3.0 mg/kg per day of a body weight of the subject. In some embodiments, the method comprises administering the compound or a pharmaceutically acceptable salt or solvent thereof is administered at a dose of at most about 0.3 mg/kg per day of a body weight of the subject. In some embodiments, the compound or a pharmaceutically acceptable salt or solvent thereof is administered at a dose of at most about 0.03 mg/kg per day of a body weight of the subject. In some embodiments, the composition for use comprises a unit dose sufficient to deliver at most about 0.3 mg/kg per day of a body weight of a subject. In some embodiments, the composition for use comprises a unit dose sufficient to deliver at most about 0.03 mg/kg per day of a body weight of the subject. In some embodiments, the subject has a fibromyalgia-associated allodynia.

Disclosed herein are methods of treating fibromyalgia comprising administering to the subject a compound that is a GABA_{A} receptor modulator. Also disclosed herein are compositions for use in treating fibromyalgia comprising a compound that is a GABA_{A} receptor modulator. In some embodiments, a compound is of the general formula (1a), general formula (1b) or general formula (1c),

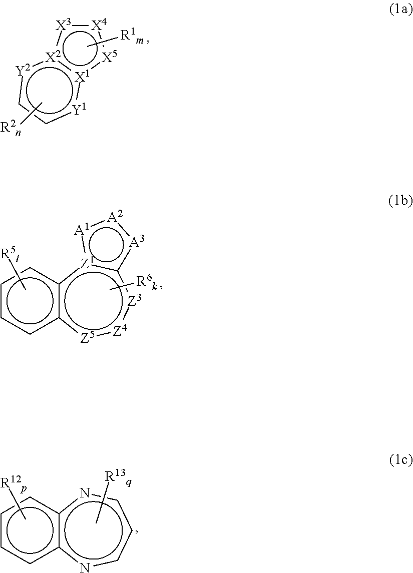

wherein X¹, X², X³, X⁴ and X⁵ are independently —C, —N, —S or —O wherein at least two of X¹, X², X³, X⁴ and X⁵ are —N, Y¹ and Y² are independently —C or —N, m of R¹ₘ is 1, wherein R¹ is an unsubstituted phenyl, a phenyl substituted with C₁-C₄-alkyl, F, Cl, Br, I, —CN, a substituted or unsubstituted biphenyl or —(C═O)—R³, wherein R³ is a substituted or unsubstituted aryl or 5- to 6-membered heteroaryl, n of R²ₙ is 1 or 2, wherein each R² is independently a substituted or unsubstituted C₃-C₈ cycloalkyl, a substituted or unsubstituted C₁-C₆ alkyl, a substituted or unsubstituted C₁-C₆ alcohol, a substituted or unsubstituted 6-membered heteroaryl, a halogen, or —O—CH₂—R⁴, wherein R⁴ is a substituted or unsubstituted 5- or 6-membered heteroaryl, Z¹, Z³, Z⁴, and Z⁵ are independently —C, —N, —S or —O, A¹, and A² and A³, are independently —C, —N, —(C═O)—O—R⁷, or

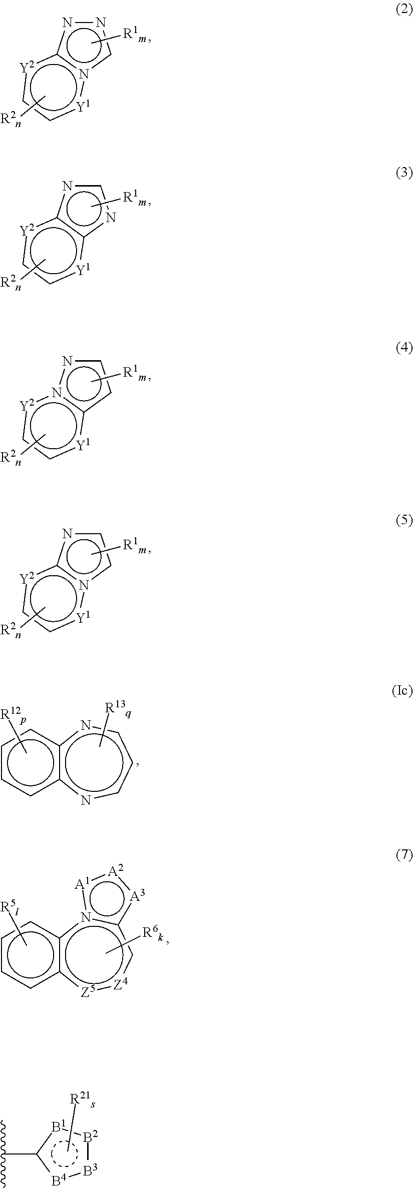

wherein R⁷ is alkyl, B¹, B², B³, and B⁴ are independently —C, —N, or —O, s of R²¹ₛ is 1, 2, 3 or 4, and R²¹ is independently hydrogen or C₁-C₆ alkyl, l of R⁵ₗ is 1 or 2, wherein each R⁵ is independently a C₁-C₄ alkinyl or a halogen, k of R⁶ₖ is 1, 2, 3 or 4, wherein each R⁶ is independently a substituted or unsubstituted aryl, a substituted or unsubstituted heteroaryl, a substituted or unsubstituted C₁-C₃ alkyl, or hydrogen, p of R¹² is 1 or 2, wherein each R¹² is independently a substituted or unsubstituted C₁-C₄-alkyl, I, Br, Cl or F, and q of R¹³ is 1, 2, 3 or 4, wherein each R¹³ is independently a substituted or unsubstituted aryl, a substituted or unsubstituted heteroaryl, a substituted or unsubstituted C₁-C₃ alkyl, oxygen or hydrogen. In some embodiments, a compound is of the general formula (2), (3), (4), (5), (1c) or (7), In some embodiments, a compound is of the general formula (2a), (3a), (4a), (5a), (5b), (1c) or (7a)

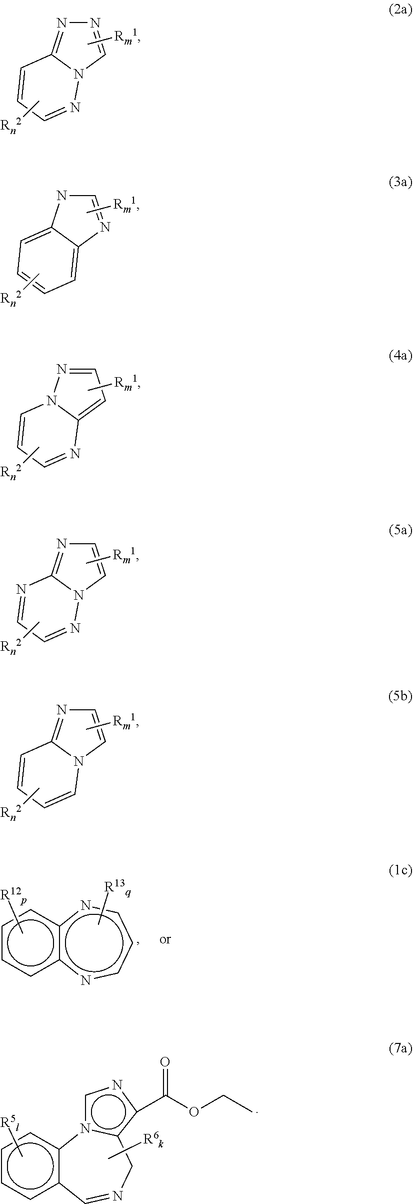

In some embodiments, m of R¹ is 1, and R¹ is: an unsubstituted phenyl, a substituted phenyl comprising C₁-C₄-alkyl, F, Cl, Br, I, or —CN as substituents, an unsubstituted biphenyl, a substituted biphenyl comprising at least one —CN as a substituent, a substituted biphenyl comprising at least one —CN as a substituent, or —(C═O)—R³, wherein R³ is pyridine. In some embodiments, a compound is of the general formula (2a′), (3a′), (4a′), (5a′), (5b′), (VI) or (7a′),

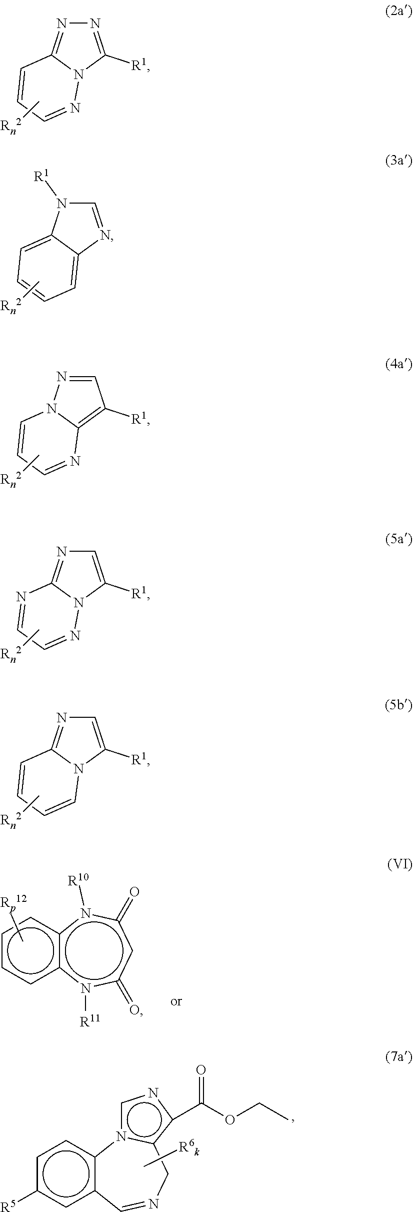

wherein R₁₀ is a substituted or unsubstituted aryl, a substituted or unsubstituted C₁-C₃ alkyl or hydrogen, R₁₁ is a substituted or unsubstituted aryl, a substituted or unsubstituted C₁-C₃ alkyl, or hydrogen, or p of R¹² is 1 and R¹² is I, Br, Cl or F. In some embodiments, a compound is of the general formula (2a″), (3a″), (4a″), (5a″), (5b″), (VIa″) or (7a″),

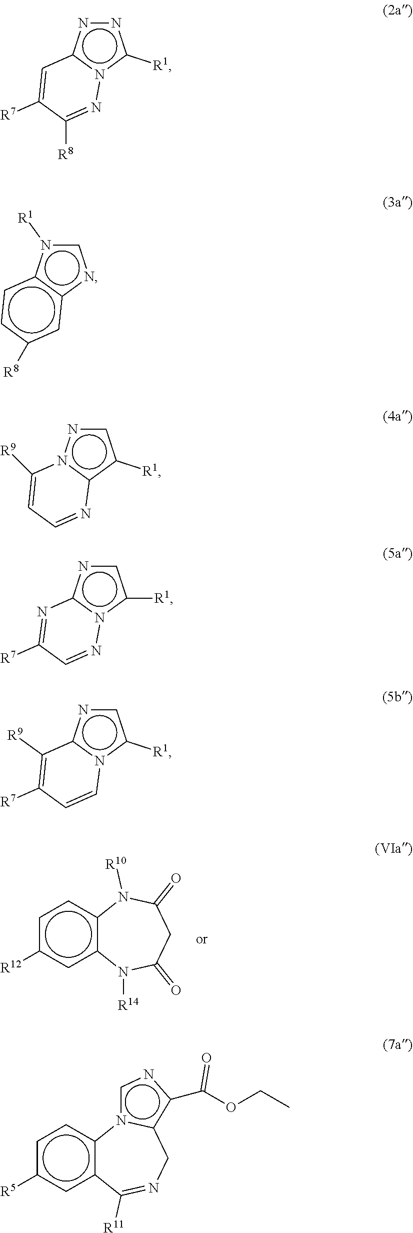

wherein R⁷ is: an unsubstituted C₁-C₆ alkyl, an unsubstituted C₃-C₈ cycloalkyl, an unsubstituted C₁-C₆ alcohol, R⁸ is: —O—CH₂—R⁴, wherein R⁴ is a substituted or unsubstituted 5-membered heteroaryl, or an unsubstituted C₁-C₆ alcohol, R⁹ is: an unsubstituted C₆ heteroaryl, or a halogen, or R⁹ is an unsubstituted 6-membered heteroaryl in formula (4a″) or a halogen in formula (5b″), R¹⁰ is a C₁-C₃ alkyl or hydrogen, R¹¹ is a substituted or unsubstituted aryl or heteroaryl, R¹⁴ is a substituted or unsubstituted aryl or heteroaryl, R¹² is I, Cl, Br or F, or R⁵ is C₂ alkinyl or I. In some embodiments, a compound is of the general formula (8):

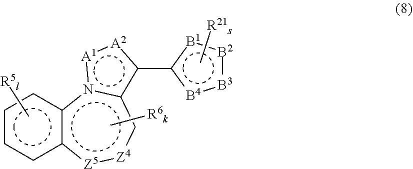

wherein Z⁴ and Z⁵ are independently —C, —N, —S or —O, A¹ and A² are independently —C, —N or —(C═O)—O—R⁷, wherein R⁷ is alkyl, B¹, B², B³, and B⁴ are independently —C, —N, or —O, l of R⁵ₗ is 1 or 2, wherein R⁵ is C₁-C₄ alkinyl or a halogen, k of R⁶ₖ is 1, 2, 3 or 4, wherein each R⁶ is independently a substituted or unsubstituted aryl, a substituted or unsubstituted heteroaryl, a substituted or unsubstituted C₁-C₃ alkyl, or hydrogen, and s of R²¹ₛ is 1, 2, 3 or 4, wherein each R²¹ is independently hydrogen or C₁-C₆ alkyl. In some embodiments, a compound is an α1, α2, α3, or α5 GABA_{A} receptor modulator. In some embodiments, a compound is a positive, allosteric α2 or α3 GABA_{A} receptor modulator. In some embodiments, a subject is a human. In some embodiments, a subject is a dog.

Also disclosed herein are methods of treating pain in a subject comprising administering to the subject a compound of the general formula (1a), general formula (1b) or general formula (1c),

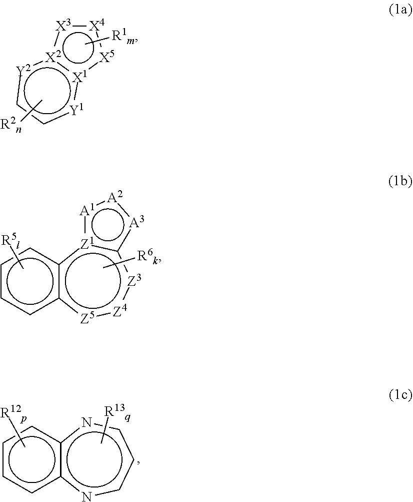

wherein X¹, X², X³, X⁴ and X⁵ are independently —C, —N, —S or —O wherein at least two of X¹, X², X³, X⁴ and X⁵ are —N, Y¹ and Y² are independently —C or —N, m of R¹ₘ is 1, wherein R¹ is an unsubstituted phenyl, a phenyl substituted with C₁-C₄-alkyl, F, Cl, Br, I, —CN, a substituted or unsubstituted biphenyl or —(C═O)—R³, wherein R³ is a substituted or unsubstituted aryl or 5- to 6-membered heteroaryl, n of R²ₙ is 1 or 2, wherein each R² is independently a substituted or unsubstituted C₃-C₈ cycloalkyl, a substituted or unsubstituted C₁-C₆ alkyl, a substituted or unsubstituted C₁-C₆ alcohol, a substituted or unsubstituted 6-membered heteroaryl, a halogen, or —O—CH₂—R⁴, wherein R⁴ is a substituted or unsubstituted 5- or 6-membered heteroaryl, Z¹, Z³, Z⁴, and Z⁵ are independently —C, —N, —S or —O, A¹, and A² and A³, are independently —C, —N, —(C═O)—O—R⁷, or

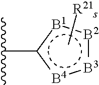

wherein R⁷ is alkyl, B¹, B², B³, and B⁴ are independently —C, —N, or —O, s of R²¹ₛ is 1, 2, 3 or 4, and R²¹ is independently hydrogen or C₁-C₆ alkyl, l of R⁵ₗ is 1 or 2, wherein each R⁵ is independently a C₁-C₄ alkinyl or a halogen, k of R⁶ₖ is 1, 2, 3 or 4, wherein each R⁶ is independently a substituted or unsubstituted aryl, a substituted or unsubstituted heteroaryl, a substituted or unsubstituted C₁-C₃ alkyl, or hydrogen, p of R¹² is 1 or 2, wherein each R¹² is independently a substituted or unsubstituted C₁-C₄-alkyl, I, Br, Cl or F, and q of R¹³ is 1, 2, 3 or 4, wherein each R¹³ is independently a substituted or unsubstituted aryl, a substituted or unsubstituted heteroaryl, a substituted or unsubstituted C₁-C₃ alkyl, oxygen or hydrogen. Also disclosed herein are compositions for use treating pain comprising the compound. In some embodiments, a compound is of the general formula (2), (3), (4), (5), (1c) or (7),

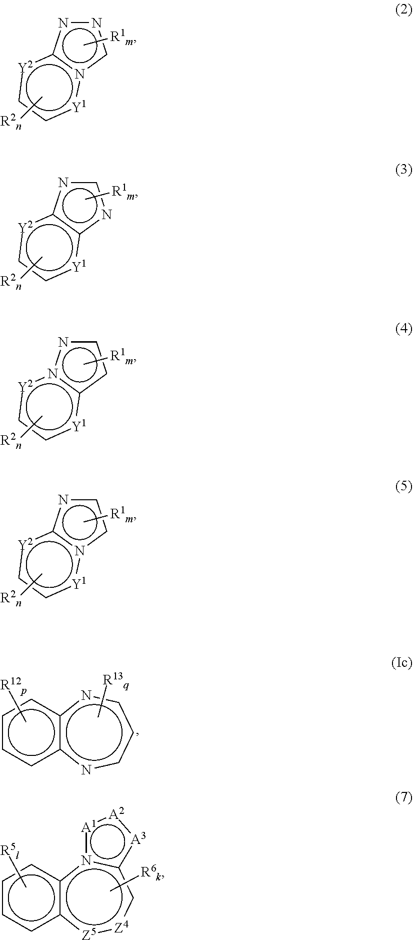

In some embodiments, a compound is of the general formula (2a), (3a), (4a), (5a), (5b), (1c) or (7a)

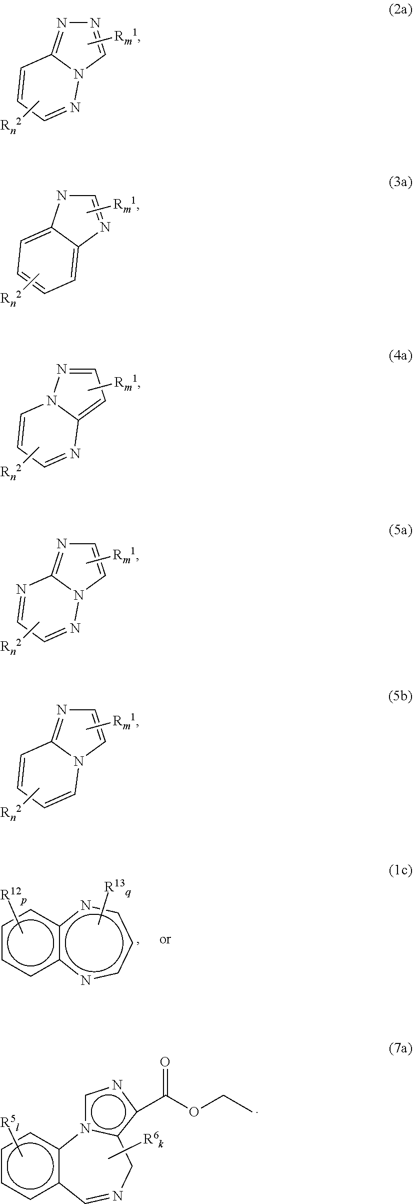

In some embodiments, m of R¹ is 1, and R¹ is: an unsubstituted phenyl, a substituted phenyl comprising C₁-C₄-alkyl, F, Cl, Br, I, or —CN as substituents, an unsubstituted biphenyl, a substituted biphenyl comprising at least one —CN as a substituent, a substituted biphenyl comprising at least one —F as a substituent, or —(C═O)—R³, wherein R³ is pyridine. In some embodiments, a compound is of the general formula (2a′), (3a′), (4a′), (5a′), (5b′), (VI) or (7a′),

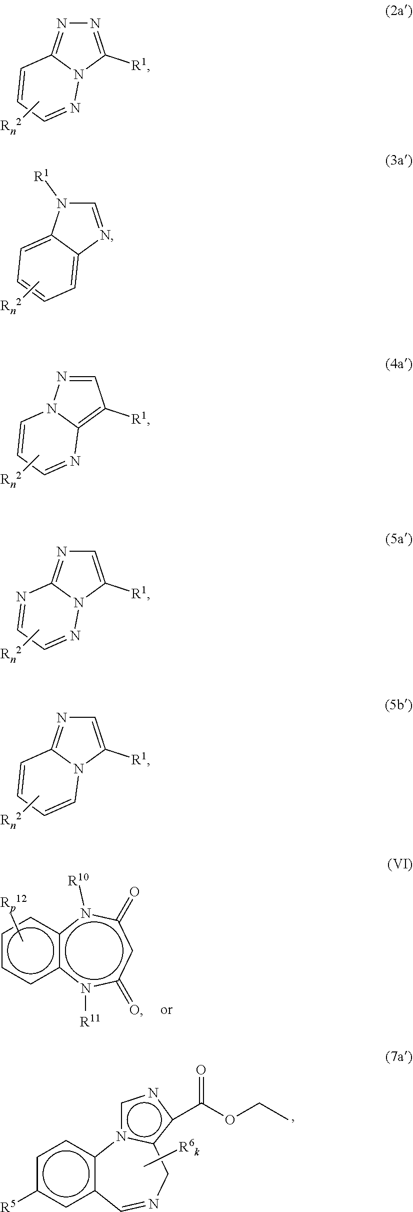

wherein R₁₀ is a substituted or unsubstituted aryl, a substituted or unsubstituted C₁-C₃ alkyl or hydrogen, R₁₁ is a substituted or unsubstituted aryl, a substituted or unsubstituted C₁-C₃ alkyl, or hydrogen, or p of R¹² is 1 and R¹² is I, Br, Cl or F. In some embodiments, a compound is of the general formula (2a″), (3a″), (4a″), (5a″), (5b″), (VIa″) or (7a″),

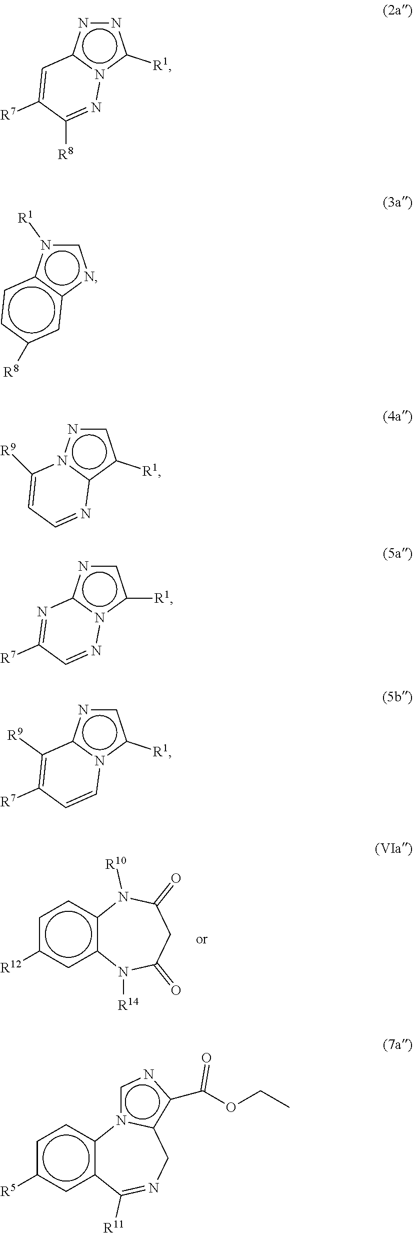

wherein R⁷ is: an unsubstituted C₁-C₆ alkyl, an unsubstituted C₃-C₈ cycloalkyl, an unsubstituted C₁-C₆ alcohol, R⁸ is: —O—CH₂—R⁴, wherein R⁴ is a substituted or unsubstituted 5-membered heteroaryl, or an unsubstituted C₁-C₆ alcohol, R⁹ is: an unsubstituted C₆ heteroaryl, or a halogen, or R⁹ is an unsubstituted 6-membered heteroaryl in formula (4a″) or a halogen in formula (5b″), R¹⁰ is a C₁-C₃ alkyl or hydrogen, R¹¹ is a substituted or unsubstituted aryl or heteroaryl, R¹⁴ is a substituted or unsubstituted aryl or heteroaryl, R¹² is I, Cl, Br or F, or R⁵ is C₂ alkinyl or I. In some embodiments, a compound is of the general formula (8):

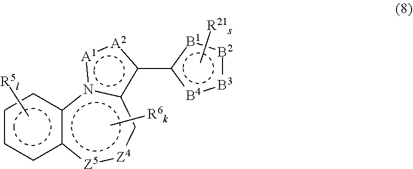

wherein Z⁴ and Z⁵ are independently —C, —N, —S or —O, A¹ and A² are independently —C, —N or —(C═O)—O—R⁷, wherein R⁷ is alkyl, B¹, B², B³, and B⁴ are independently —C, —N, or —O, l of R⁵ₗ is 1 or 2, wherein R⁵ is C₁-C₄ alkinyl or a halogen, k of R⁶ₖ is 1, 2, 3 or 4, wherein each R⁶ is independently a substituted or unsubstituted aryl, a substituted or unsubstituted heteroaryl, a substituted or unsubstituted C₁-C₃ alkyl, or hydrogen, and s of R²¹ₛ is 1, 2, 3 or 4, wherein each R²¹ is independently hydrogen or C₁-C₆ alkyl. In some embodiments, a compound is an α1, α2, α3, or α5 GABA_{A} receptor modulator. In some embodiments, a compound is a positive, allosteric α2 or α3 GABA_{A} receptor modulator. In some embodiments, a subject is a human. In some embodiments, a subject is a dog.

Also disclosed herein are methods of treating allodynia in a subject comprising administering to the subject a compound of the general formula (1a), general formula (1b) or general formula (1c),

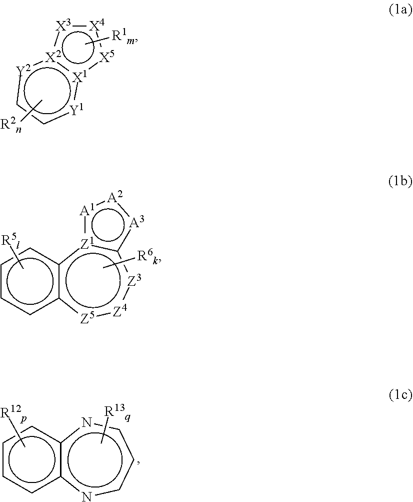

wherein X¹, X², X³, X⁴ and X⁵ are independently —C, —N, —S or —O wherein at least two of X¹, X², X³, X⁴ and X⁵ are —N, Y¹ and Y² are independently —C or —N, m of R¹ₘ is 1, wherein R¹ is an unsubstituted phenyl, a phenyl substituted with C₁-C₄-alkyl, F, Cl, Br, I, —CN, a substituted or unsubstituted biphenyl or —(C═O)—R³, wherein R³ is a substituted or unsubstituted aryl or 5- to 6-membered heteroaryl, n of R²ₙ is 1 or 2, wherein each R² is independently a substituted or unsubstituted C₃-C₈ cycloalkyl, a substituted or unsubstituted C₁-C₆ alkyl, a substituted or unsubstituted C₁-C₆ alcohol, a substituted or unsubstituted 6-membered heteroaryl, a halogen, or —O—CH₂—R⁴, wherein R⁴ is a substituted or unsubstituted 5- or 6-membered heteroaryl, Z¹, Z³, Z⁴, and Z⁵ are independently —C, —N, —S or —O, A¹, and A² and A³, are independently —C, —N, —(C═O)—O—R⁷, or

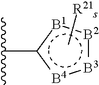

wherein R⁷ is alkyl, B¹, B², B³, and B⁴ are independently —C, —N, or —O, s of R²¹ₛ is 1, 2, 3 or 4, and R²¹ is independently hydrogen or C₁-C₆ alkyl, l of R⁵ₗ is 1 or 2, wherein each R⁵ is independently a C₁-C₄ alkinyl or a halogen, k of R⁶ₖ is 1, 2, 3 or 4, wherein each R⁶ is independently a substituted or unsubstituted aryl, a substituted or unsubstituted heteroaryl, a substituted or unsubstituted C₁-C₃ alkyl, or hydrogen, p of R¹² is 1 or 2, wherein each R¹² is independently a substituted or unsubstituted C₁-C₄-alkyl, I, Br, Cl or F, and q of R¹³ is 1, 2, 3 or 4, wherein each R¹³ is independently a substituted or unsubstituted aryl, a substituted or unsubstituted heteroaryl, a substituted or unsubstituted C₁-C₃ alkyl, oxygen or hydrogen. Also disclosed herein are compositions for use in treating allodynia comprising the compound. In some embodiments, a compound is of the general formula (2), (3), (4), (5), (1c) or (7),

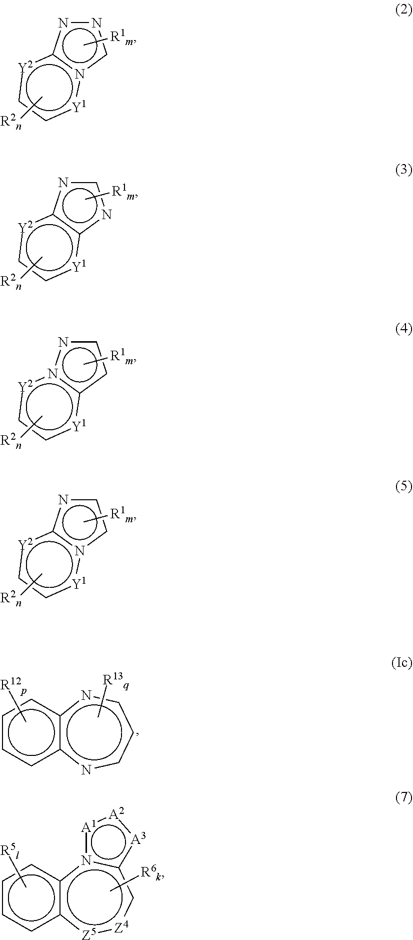

In some embodiments, a compound is of the general formula (2a), (3a), (4a), (5a), (5b), (1c) or (7a)

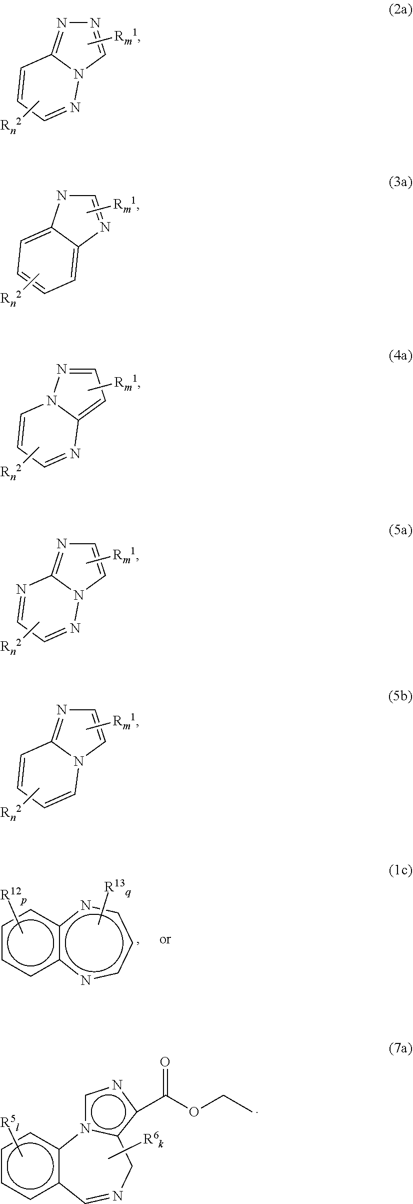

In some embodiments, m of R′ is 1, and R′ is: an unsubstituted phenyl, a substituted phenyl comprising C₁-C₄-alkyl, F, Cl, Br, I, or —CN as substituents, an unsubstituted biphenyl, a substituted biphenyl comprising at least one —CN as a substituent, a substituted biphenyl comprising at least one —F as a substituent, or —(C═O)—R³, wherein R³ is pyridine. In some embodiments, a compound is of the general formula (2a′), (3a′), (4a′), (5a′), (5b′), (VI) or (7a′),

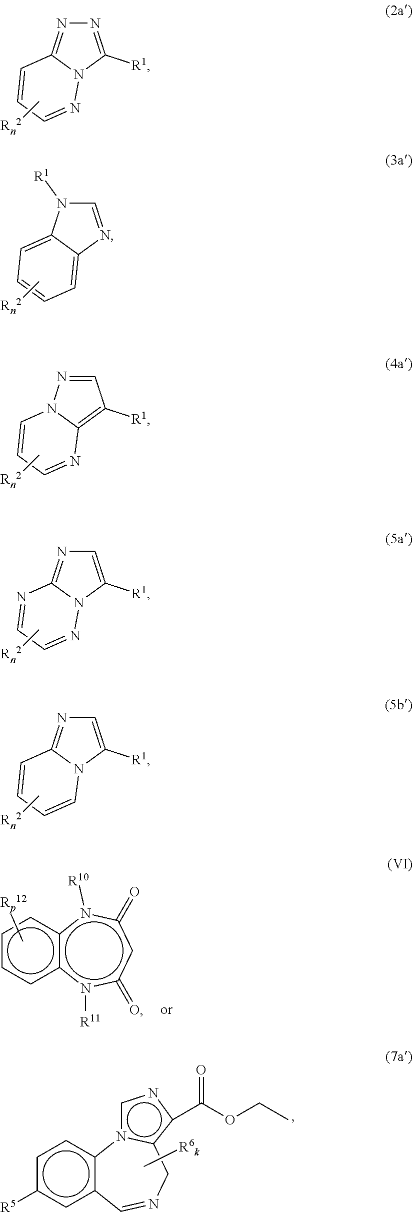

wherein R₁₀ is a substituted or unsubstituted aryl, a substituted or unsubstituted C₁-C₃ alkyl or hydrogen, R₁₁ is a substituted or unsubstituted aryl, a substituted or unsubstituted C₁-C₃ alkyl, or hydrogen, or p of R¹² is 1 and R¹² is I, Br, Cl or F. In some embodiments, a compound is of the general formula (2a″), (3a″), (4a″), (5a″), (5b″), (VIa″) or (7a″),

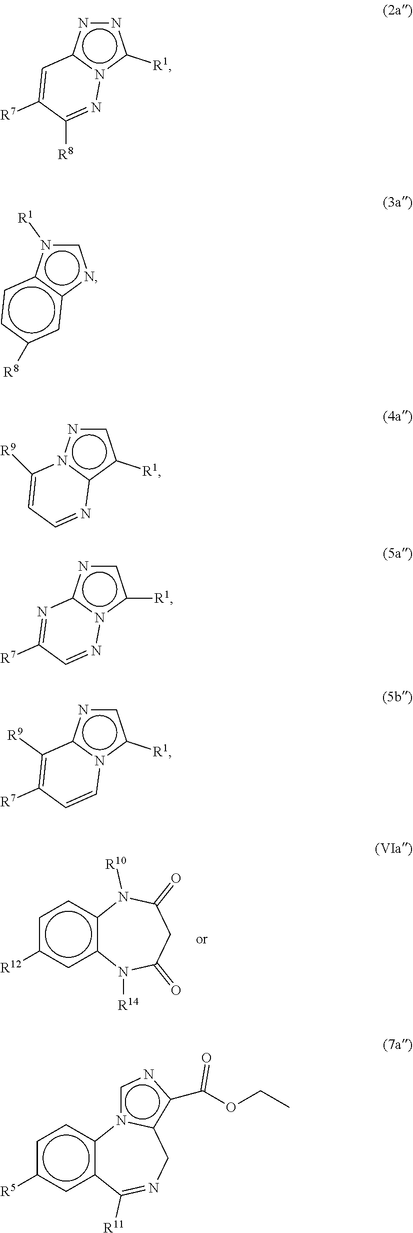

wherein R⁷ is: an unsubstituted C₁-C₆ alkyl, an unsubstituted C₃-C₈ cycloalkyl, an unsubstituted C₁-C₆ alcohol, R⁸ is: —O—CH₂—R⁴, wherein R⁴ is a substituted or unsubstituted 5-membered heteroaryl, or an unsubstituted C₁-C₆ alcohol, R⁹ is: an unsubstituted C₆ heteroaryl, or a halogen, or R⁹ is an unsubstituted 6-membered heteroaryl in formula (4a″) or a halogen in formula (5b″), R¹⁰ is a C₁-C₃ alkyl or hydrogen, R¹¹ is a substituted or unsubstituted aryl or heteroaryl, R¹⁴ is a substituted or unsubstituted aryl or heteroaryl, R¹² is I, Cl, Br or F, or R⁵ is C₂ alkinyl or I. In some embodiments, a compound is of the general formula (8):

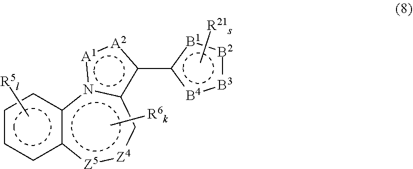

wherein Z⁴ and Z⁵ are independently —C, —N, —S or —O, A¹ and A² are independently —C, —N or —(C═O)—O—R⁷, wherein R⁷ is alkyl, B¹, B², B³, and B⁴ are independently —C, —N, or —O, l of R⁵ₗ is 1 or 2, wherein R⁵ is C₁-C₄ alkinyl or a halogen, k of R⁶ₖ is 1, 2, 3 or 4, wherein each R⁶ is independently a substituted or unsubstituted aryl, a substituted or unsubstituted heteroaryl, a substituted or unsubstituted C₁-C₃ alkyl, or hydrogen, and s of R²¹ₛ is 1, 2, 3 or 4, wherein each R²¹ is independently hydrogen or C₁-C₆ alkyl. In some embodiments, a compound is an α1, α2, α3, or α5 GABA_{A} receptor modulator. In some embodiments, a compound is a positive, allosteric α2 or α3 GABA_{A} receptor modulator. In some embodiments, a subject is a human. In some embodiments, a subject is a dog.

Also disclosed herein are methods of treating pain in a subject comprising administering to the subject an amount of a pharmaceutical composition comprising a compound selected from the group consisting of:

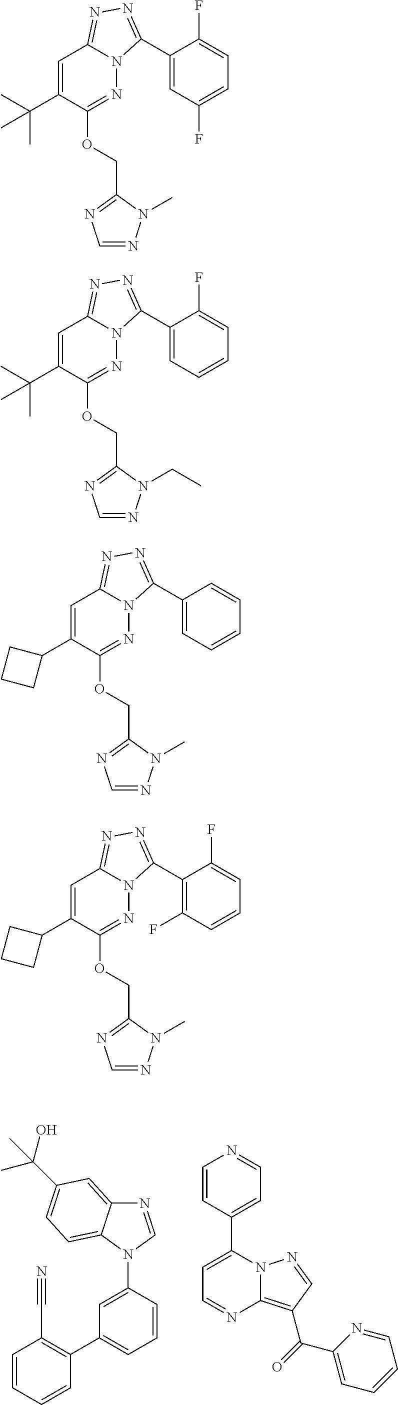

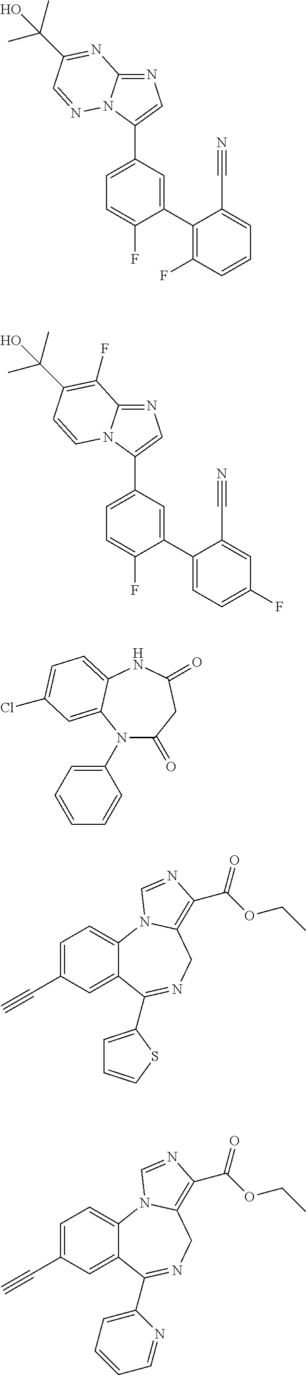

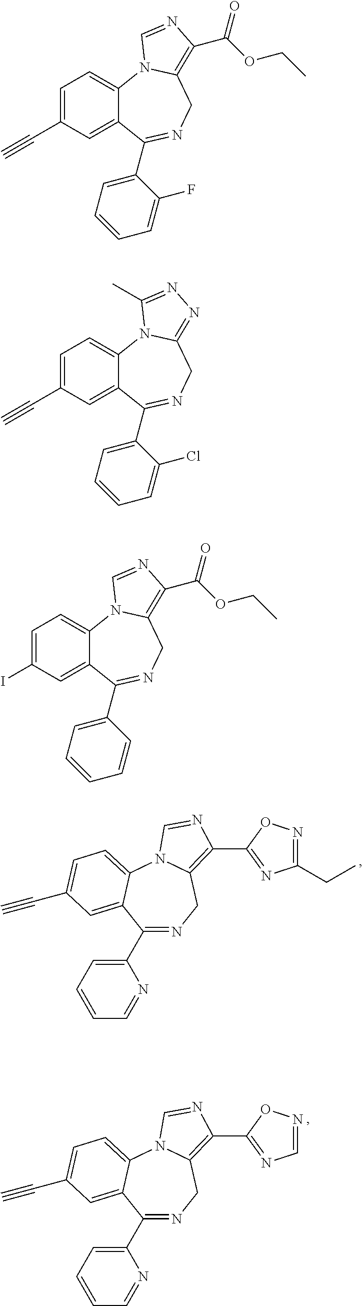

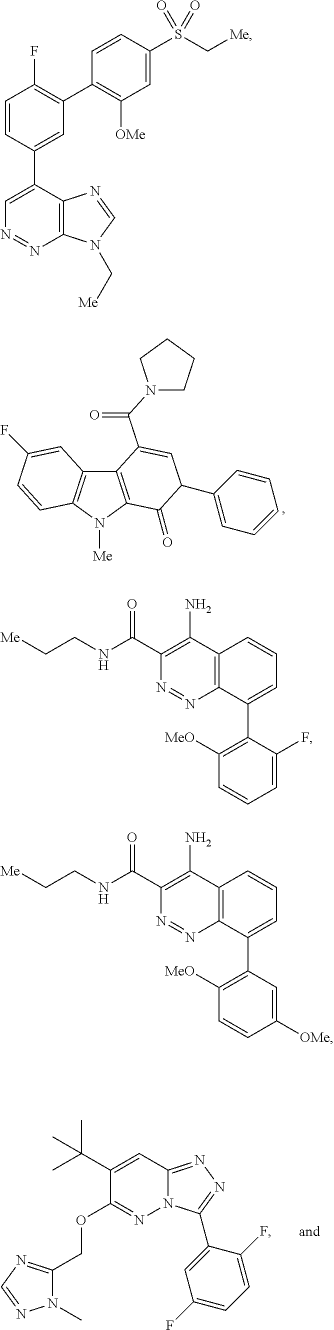 [SMILES: CCn1cnc2c(-c3ccc(F)c(-c4ccc(S(=O)(=O)CC)cc4OC)c3)cnnc21 ; Cn1c2c(c3cc(F)ccc31)C(C(=O)N1CCCC1)=CC(c1ccccc1)C2=O ; CCCNC(=O)c1nnc2c(-c3c(F)cccc3OC)cccc2c1N ; CCCNC(=O)c1nnc2c(-c3cc(OC)ccc3OC)cccc2c1N ; Cn1ncnc1COc1nn2c(-c3cc(F)ccc3F)nnc2cc1C(C)(C)C ; "and"]

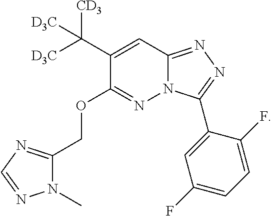 [SMILES: [2H]C([2H])([2H])C(c1cc2nnc(-c3cc(F)ccc3F)n2nc1OCc1ncnn1C)(C([2H])([2H])[2H])C([2H])([2H])[2H]]

Also disclosed herein are compositions for use in treating pain comprising the compound. In some embodiments, a compound is:

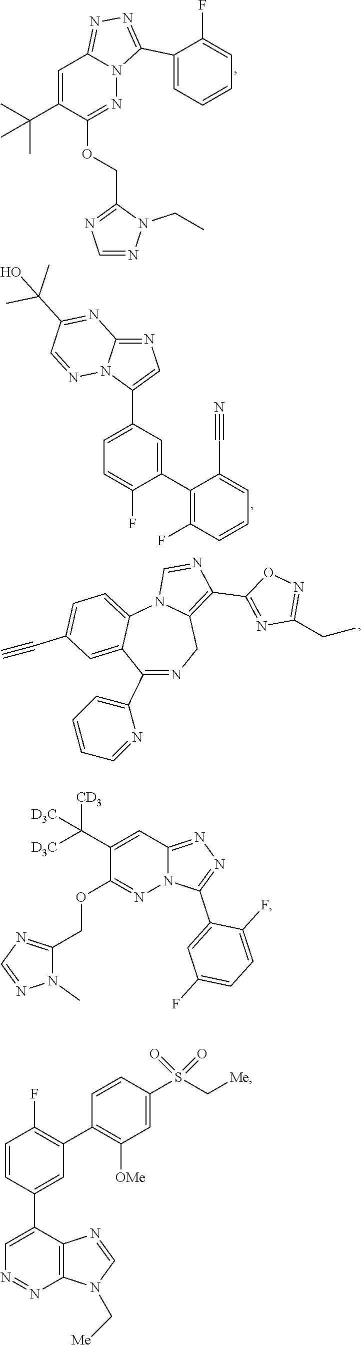

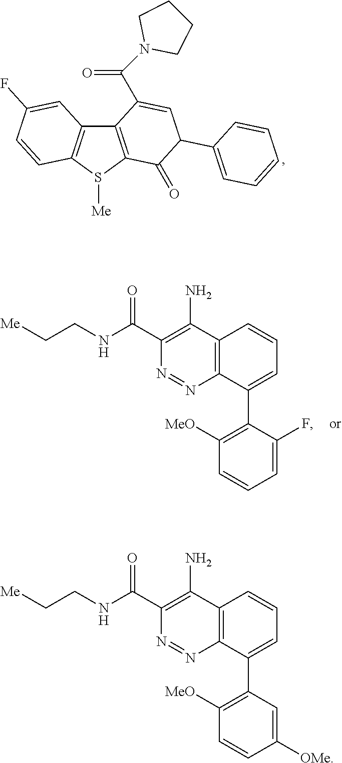

In some embodiments, an amount is effective to treat the pain when administered at a dose of from about 0.3 mg/kg per day to about 3.0 mg/kg per day of a body weight of a subject. In some embodiments, pain is related to central nervous system sensitization. In some embodiments, pain is chronic. In some embodiments, a subject is a human. In some embodiments, a subject is a dog. In some embodiments, a pharmaceutical composition or composition for use further comprises a pharmaceutically acceptable excipient, diluent, or carrier. In some embodiments, a pharmaceutical composition or composition for use comprises a carrier, wherein the carrier is methyl cellulose. In some embodiments, an administering comprises an oral administration. In some embodiments, an administering is performed at least once a day.

Also disclosed herein are methods of treating fibromyalgia, allodynia, hypersensitivity, or pain associated with central nervous system sensitization in a subject in need thereof comprising administering to the subject a pharmaceutical composition comprising a compound of:

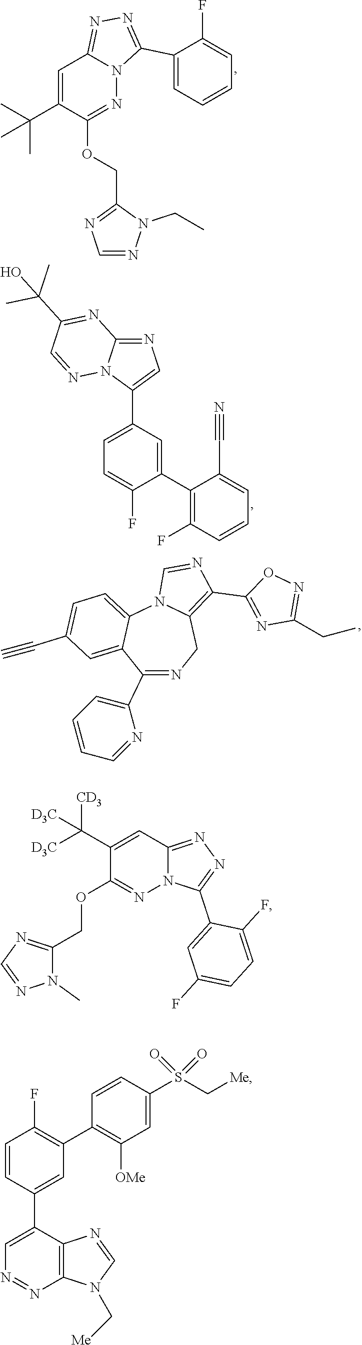

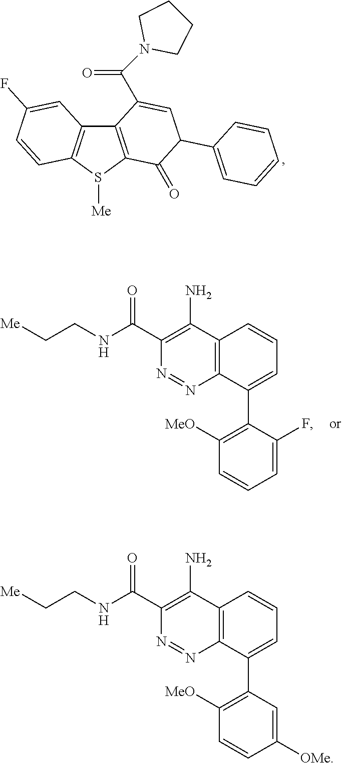

Also disclosed herein are compositions for use in treating fibromyalgia, allodynia, hypersensitivity, or pain comprising the compound.

Further described herein are GABA_{A} receptor modulators or pharmaceutical compositions comprising the modulators for use in the treatment of fibromyalgia, allodynia, hypersensitivity, or pain associated with central nervous system sensitization in a subject in need thereof. In some embodiments, described herein are compounds, pharmaceutically acceptable salts or solvates thereof, and pharmaceutical compositions comprising the compounds, salts, or solvates, for use in the treatment of fibromyalgia, allodynia, hypersensitivity, or pain associated with central nervous system sensitization in a subject in need thereof. In some embodiments, described herein is Compound 1, or a pharmaceutically acceptable salt or solvate thereof, for use in the treatment of fibromyalgia.

### BRIEF DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which the principles of exemplary embodiments are utilized, and the accompanying drawings of which:

FIG. 1 depicts development of reserpine-induced mechanical allodynia in an animal model. Mean±SEM ipsilateral and contralateral paw response thresholds were plotted following reserpine injection in vehicle treated animals. All animals received 0.5% methylcellulose (5 mL/kg) via oral gavage (n=10). The legend “++” refers to a p value of less than 0.01. The p value is predictive of the probability of the null hypothesis occurring.

FIG. 2 depicts the effect of GABA_{A} receptor modulators on reserpine induced persistent pain. Mean±SEM mean response thresholds following reserpine induced persistent pain in vehicle and treated animals during the pharmacological assessment period (day 0). All animals received 0.5% methylcellulose (5 mL/kg) or a GABA_{A} receptor modulator (Compound 1, 0.3 or 3 mg/kg) via oral gavage (n=10). The legend “++” refers to a p value of less than 0.01; and the legend “+” refers to a p value of less than 0.05. The p value is predictive of the probability of the null hypothesis occurring.

FIG. 3 depicts the effect of pregabalin on reserpine-induced persistent pain. Mean±SEM ipsilateral and contralateral paw response thresholds following reserpine injection in vehicle and positive control-treated animals. All animals received 0.5% methylcellulose (5 mL/kg) or pregabalin (30 mg/kg) via oral gavage (n=10). The legend “+,” “++,” “+++,” and “++++” refer to a p value of less than 0.05, less than 0.01, less than 0.001, and less than 0.0001, respectively. The p value is predictive of the probability of the null hypothesis occurring.

FIG. 4 depicts exemplary GABA_{A} receptor modulators.

### DETAILED DESCRIPTION

### Definitions

The term “GABA receptor” refers to receptors of the inhibitory neurotransmitter gamma-aminobutyric acid (GABA). GABA_{A} receptors (ionotropic receptors) are ligand-gated ion channels and GABA_{B} receptors, also known as metabotropic receptors, are G protein-coupled receptors. GABA_{A} receptors are the most common and most important inhibitory receptors within the central nervous system. GABA_{A} receptors comprise five subunits that are grouped in eight classes: α₁₋₆, β₁₋₃, γ₁₋₃, δ, ε, π, θ and ρ₁₋₃. The majority of GABA_{A} receptors comprise two α, two β and one γ subunit. Compounds such as benzodiazepines can bind to sites distinct from the endogenous ligand GABA_{A} For example, a benzodiazepine can bind to a binding site situated between the α and γ subunits. A compound described herein can be an allosteric modulator of a GABA_{A} receptor. The different types of a subunits confer different properties to the GABA_{A} receptor. Whereas the α1 subunit is among other functions responsible for the sedative effect of benzodiazepines, the α2 subunit is connected, among other things, to the anxiolytic function of the receptors and the α3 subunit confers, among other things, the muscle relaxing properties of the GABA_{A} receptor.

As used herein, the term “GABA_{A} receptor modulator” refers to a substance that modulates the activities of GABA_{A} receptors, provided that a GABA_{A} receptor modulator is not gamma-aminobutyric acid. A positive GABA_{A} receptor modulator increases the activity of GABA_{A} receptor protein. A negative GABA_{A} receptor modulator decreases the activity of GABA_{A} receptor protein.

The term “allosteric modulator” refers to a substance that indirectly modulates the effects of an agonist at a receptor. A positive allosteric modulator induces an amplification of the agonist's effect, without having an effect by itself in the absence of the agonist. An allosteric modulator can bind to a site distinct from the agonist's binding site (allosteric). A negative allosteric modulator reduces the agonist's effect at a receptor, without having an effect by itself in the absence of the agonist.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value.

The term “subject”, “patient” or “individual” as used herein encompasses a mammal and a non-mammal. A mammal can be any member of the Mammalian class, including but not limited to a human, a non-human primates such as a chimpanzee, an ape or other monkey species; a farm animal such as cattle, a horse, a sheep, a goat, a swine; a domestic animal such as a rabbit, a dog (or a canine), and a cat (or a feline); a laboratory animal including a rodent, such as a rat, a mouse and a guinea pig, and the like. A non-mammal can include a bird, a fish and the like. In some embodiments, a subject can be a mammal. In some embodiments, a subject can be a human. In some instances, a human can be an adult. In some instances, a human can be a child. In some instances, a human can be age 0-17 years old. In some instances, a human can be age 18-130 years old. In some instances, a subject can be a male. In some instances, a subject can be a female. In some instances, a subject can be diagnosed with, or can be suspected of having, a condition or disease. In some instances a disease or condition can be pain or a condition associated with pain. A subject can be a patient. A subject can be an individual. In some instances, a subject, patient or individual can be used interchangeably.

The terms “treat,” “treating”, “treatment,” “ameliorate” or “ameliorating” and other grammatical equivalents as used herein, include alleviating, or abating a disease or condition symptoms, inhibiting a disease or condition, e.g., arresting the development of a disease or condition, relieving a disease or condition, causing regression of a disease or condition, relieving a condition caused by the disease or condition, or stopping symptoms of a disease or condition.

A “C₁-C₆ alkyl” refers to a saturated linear or branched hydrocarbon having 1, 2, 3, 4, 5 or 6 carbon atoms, wherein one carbon-carbon bond may be unsaturated and one CH₂ moiety may be exchanged for oxygen (ether bridge). Non-limiting examples for a C₁-C₄ alkyl are methyl, ethyl, propyl, prop-2-enyl, n-butyl, 2-methylpropyl, tert-butyl, but-3-enyl, prop-2-inyl and but-3-inyl.

The term “aryl” refers to a cyclic aromatic C₅-C₁₀ hydrocarbon. Examples of aryl can include, without being restricted to, phenyl, naphthyl and heteroaryl. A heteroaryl can refer to an aryl that comprises one or several nitrogen, oxygen and/or sulfur atoms. Examples for heteroaryl can include, without being restricted to, pyrrole, thiophene, furan, imidazole, pyrazole, thiazole, oxazole, pyridine, pyrimidine, thiazin, quinoline, benzofuran and indole. An aryl or a heteroaryl can be substituted by one or more alkyl groups. The term “aryl” can refer to a hydrocarbon with alternating double and single bonds between the carbon atoms forming a ring structure (in the following an “aromatic hydrocarbon”).

The term “heteroaryl” refers to aryl compounds in which at least one ring carbon atom is replaced with an oxygen, a nitrogen or a sulfur atom. The aromatic hydrocarbon may be neutral or charged. Examples of aryl or hetero aryl groups can include benzene, pyridine, pyrrole or cyclopenta-1,3-diene-anion. Aryl or hetero aryl groups as used herein may optionally include further substituent groups.

The term “substituted,” “substituent” or the like, unless otherwise indicated, refers to the replacement of one or more hydrogen radicals in a given structure with the radical of a specified substituent including, but not limited to: halo, alkyl, alkenyl, alkynyl, aryl, heterocyclyl, thiol, alkylthio, oxo, thioxy, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and an aliphatic group. It is understood that the substituent may be further substituted.

The term “unsubstituted” means that the specified group bears no substituents. The term “optionally substituted” means that the specified group is unsubstituted or substituted by one or more substituents, independently chosen from the group of possible substituents. When indicating the number of substituents, the term “one or more” means from one substituent to the highest possible number of substitution, i.e. replacement of one hydrogen up to replacement of all hydrogens by substituents.

As used herein, C₁-Cₓ (or C₁₋ₓ) includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

Conditions

Disclosed herein are compounds for treatment of pain and associated conditions with heightened pain response. Such conditions can be chronic or acute. In some cases, a condition described herein can be triggered by external stimuli such as psychological stress, trauma, infections, or can be genetically inheritable. A condition can result from a sensitization of secondary pain neurons mediated by increased release of pro-inflammatory cytokines or nitric oxide by glial cells. In some cases, altered dopaminergic or noradrenergic signaling can produce a condition described herein. Some subjects suffering from a condition described herein may have increased levels of plasma cortisol, a decreased vasoconstriction response, decreased N-acetylaspartic acid, chronic inflammation due to increased levels of pro-inflammatory cytokines, heightened sensory sensations, disrupted sleep, insomnia, depression, numbness, concentration or memory problems, anxiety, morning stiffness, tingling in extremities, headaches, irritable bowel syndrome, painful urination, fatigue, and combinations thereof.

Compounds described herein (e.g. GABA_{A} receptor modulators) can be used to at least partially ameliorate conditions associated with pain. In some cases, a condition that is associated with pain can include acute pain, addiction, advanced prostate cancer, AIDS-related pain, allodynia, ankylosing spondylitis, arachnoiditis, arthritis, arthrofibrosis, ataxic cerebral palsy, autoimmune atrophic gastritis, autoimmune diseases, avascular necrosis, back pain, Behcet's Disease (Syndrome), breakthrough pain, burning mouth syndrome, bursitis, CADASIL, cancer pain, carpal tunnel, Cauda Equina Syndrome, central pain syndrome, cerebral palsy, cerebrospinal fluid leaks, cervical stenosis, Charcot-Marie-Tooth Disease, chronic fatigue syndrome, chronic functional abdominal pain, chronic pain, chronic pancreatitis, coccyx, collapsed lung (pneumothorax), complementary and alternative medicine, complex regional pain syndrome, corneal neuropathic pain, Crohn's Disease, degenerative disc disease, depression, Dercum's Disease, dermatomyositis, diabetic peripheral neuropathy, dystonia, Ehlers-Danlos Syndrome, endometriosis, Eosinophilia-Myalgia Syndrome, erythromelalgia Failed Back Surgery Syndrome (FBSS), fibromyalgia, gout, headaches, herniated disc, hydrocephalus, intercostal neuraligia, interstitial cystitis, irritable bowel syndrome (IBS), Juvenile Dermatositis, injury, leg pain, Loin Pain-Haematuria Syndrome, lupus, Lyme Disease, medullary sponge kidney, meralgia paresthetica, mesothelioma, migraine, mitochondrial disorders, multiple sclerosis, musculoskeletal pain, myofascial pain, myositis, neck pain, neuropathic pain, occipital neuralgia, osteoarthritis, Paget's Disease, Parkinson's Disease, Parsonage Turner Syndrome, pelvic pain, peripheral neuropathy, phantom limb pain, pinched nerve, polycystic kidney disease, polymyalgia rhuematica, polymyositis, porphyria, post herniorraphy pain syndrome, post mastectomy pain syndrome, post stroke pain, post thorocotomy pain syndrome, postherpetic neuralgia, post-polio syndrome, post-traumatic stress disorder (PTSD), primary lateral sclerosis, psoriatic arthritis, pudendal neuralgia, radiculopathy, Raynaud's Disease, restless leg syndrome, rheumatoid arthritis, sacroiliac joint dysfunction, sarcoidosis, Scheuemann's Kyphosis Disease, sciatica, scoliosis, shingles (Herpes Zoster), sickle cell anemia, Sjogren's Syndrome, sleep apnea, spasmodic torticollis, sphincter of oddi dysfunction, spinal cerebellum ataxia, spinal cord injury, spinal stenosis, syringomyelia, tarlov cysts, Tethered Cord Syndrome, Thoracic Outlet Syndrome, TMJ, transverse myelitis, trigeminal neuralgia, trigger points, ulcerative colitis, vascular pain, visceral hypersensitivity, vulvodynia, and whiplasharthritis.

In some cases, a condition can be a hypersensitivity reaction. A hypersensitivity reaction can be a type I, type II, type III, or type IV hypersensitivity reaction. A type I hypersensitivity reaction can include anaphylaxis, a drug reaction, a food allergy, an insect venon allergy, and the like. A type II hypersensitivity reaction can include an acute hemolytic transfusion reaction, autoimmune hemolytic anemia, bullous pemphigoid drug-induced neutropenia, goodpasture syndrome, Graves disease, hemolytic disease of the fetus or newborn, immune thrombocytopenia, hyperacute transplant rejection, myasthenia gravis, pernicious anemia, pemphigus vulgaris, rheumatic fever, and the like. A type III hypersensitivity reaction can include arthus reaction, drug-induced hypersensitivity vasculitis, hypersensitivity pneumonitis, polyarteritis nodosa, poststreptococcal plomerulonephritis, lupus nephritis, serum sickness, systemic lupus erythematosus, and the like. A type IV hypersensitivity reaction can include acute or chronic transplant rejection, contact dermatitis, Stevens-Johnson Syndrome, Graft-versus-host disease, Guillain-Barre syndrome, Hashimoto's thyroiditis, multiple sclerosis, rheumatoid arthritis, type-1 diabetes mellitus, and the like.

In some cases, a condition associated with pain treatable with a compound described herein is fibromyalgia. A subject suffering from fibromyalgia that is treatable with a compound described herein may have additional associated conditions, such as depression, anxiety, migraine, allodynia, pelvic pain, headache, IBS, TMJ, tinnitus, gastrointestinal reflux disease, high blood pressure, or other conditions associated with pain as described herein.

In some cases, a condition associated with pain treatable with a compound described herein is allodynia. Allodynia can occur with or without other conditions as described herein such as fibromyalgia. In some embodiments, the condition is fibromyalgia-associated allodynia. In some embodiments, the fibromyalgia-associated allodynia is different from allodynia associated with other indications. In some embodiments, the fibromyalgia-associated allodynia is resistant to therapies that are effective in treating other types of allodynia.

Compounds

Disclosed herein are compounds for treatment of pain and associated conditions. In some cases, a compound for treatment of pain and associated conditions can be a GABA_{A} receptor modulator. In certain embodiments, the compound is a positive allosteric α2 and/or α3 GABA_{A} receptor modulator.

A GABA_{A} receptor modulator that can be used in the treatment of pain and associated conditions can be a compound of general formula (1a), (1b), (1c), or a pharmaceutically acceptable salt or solvate thereof:

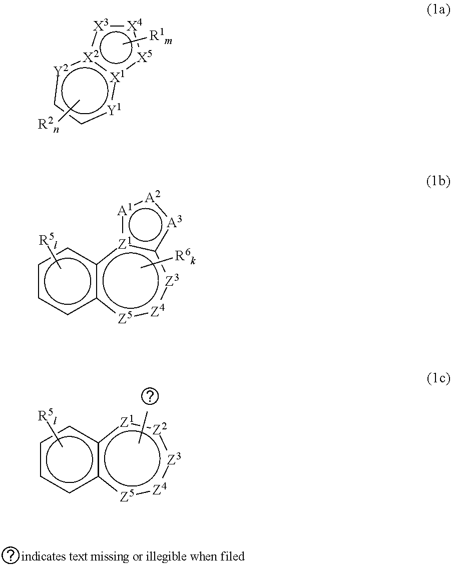

where
X¹, X², X³, X⁴ and X⁵ are independently from each other —C, —N, —S or —O wherein at least two of X¹, X², X³, X⁴ and X⁵ are —N,
Y¹ and Y² are independently from each other —C or —N,
m of R¹ₘ is 1,
R¹ is a substituted or unsubstituted C₆ aryl or —(C═O)—R³, with R³ being a substituted or unsubstituted C₆ heteroaryl,
n of R²ₙ is 1 or 2,
each R² independently from any other R² is a substituted or unsubstituted C₃-C₈ cycloalkyl, a substituted or unsubstituted C₁-C₆ alkyl, a substituted or unsubstituted C₁-C₆ alcohol, a substituted or unsubstituted C₆ heteroaryl, a halogen, in some instances —F, or —O—CH₂—R⁴, with R⁴ being a substituted or unsubstituted C₄ heteroaryl,
Z¹, Z², Z³, Z⁴ and Z⁵ are independently of each other —C, —N, —S or —O,
A¹, and A² and A³, are independently of each other —C, —N, —(C═O)—O—R⁷, or

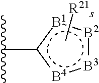

where R⁷ is alkyl,
B¹, B², B³, and B⁴ are independently of each other —C, —N, or —O,
s of R²¹ₛ is 1, 2, 3 or 4, and
each R²¹ is independently hydrogen or C₁-C₆ alkyl,
l of R⁵ₗ is 1 or 2,
each R⁵ is independently from each other a C₁-C₄ alkinyl or a halogen, in some instances —Cl,
k of R⁶ₖ is 1, 2, 3 or 4, in some instances 1 or 2,
each R⁶ is independently from each other a substituted or unsubstituted aryl, a substituted or unsubstituted heteroaryl, a substituted or unsubstituted C₁-C₃ alkyl, oxygen or hydrogen.

In certain embodiments a compound comprising the general formula (1a) is provided, wherein X¹, X², X³, X⁴ and X⁵ are independently from each other —C, —N, —S or —O wherein at least two of X¹, X², X³, X⁴ and X⁵ are —N, Y¹ and Y² are independently from each other —C or —N, m of R¹ₘ is 1, R¹ is a substituted or unsubstituted C₆ aryl or —(C═O)—R³, with R³ being a substituted or unsubstituted C₆ heteroaryl, n of R²ₙ is 1 or 2, each R² independently from any other R² is a substituted or unsubstituted C₃-C₈ cycloalkyl, a substituted or unsubstituted C₁-C₆ alkyl, a substituted or unsubstituted C₁-C₆ alcohol, a substituted or unsubstituted C₆ heteroaryl, a halogen, in some instances —F, or —O—CH₂—R⁴, with R⁴ being a substituted or unsubstituted C₄ heteroaryl.

In certain embodiments, a compound comprising the general formula (1b) is provided, wherein Z³, Z⁴ and Z⁵ are independently of each other —C, —N, —S or —O, A¹, A² and A³, are independently of each other —C, —N or —(C═O)—O—R⁷, with R⁷ being an alkyl, l of R⁵ₗ is 1 or 2, each R⁵ is independently from each other a C₁-C₄ alkinyl or a halogen, in some instances —Cl, k of R⁶ₖ is 1, 2, 3 or 4, in some instances 1 or 2, each R⁶ is independently from each other a substituted or unsubstituted aryl, a substituted or unsubstituted heteroaryl, a substituted or unsubstituted C₁-C₃ alkyl, oxygen or hydrogen.

In certain embodiments, a compound comprising the general formula (1c) is provided, wherein Z¹, Z², Z³, Z⁴ and Z⁵ are independently of each other —C, —N, —S or —O, l of R⁵ₗ is 1 or 2, each R⁵ is independently from each other a C₁-C₄ alkinyl or a halogen, in some instances —Cl, k of R⁶ₖ is 1, 2, 3 or 4, in some instances 1 or 2, each R⁶ is independently from each other a substituted or unsubstituted aryl, a substituted or unsubstituted heteroaryl, a substituted or unsubstituted C₁-C₃ alkyl, oxygen or hydrogen.

In certain embodiments, the compound comprises the general formula (1a), general formula (1b) or general formula (1c), wherein X¹, X², X³, X⁴ and X⁵ are independently from each other —C, —N, wherein at least two of X¹, X², X³, X⁴ and X⁵ are —N, Y¹ and Y² are independently from each other —C or —N, m of R¹ₘ is 1, R¹ is a substituted or unsubstituted C₆ aryl or —(C═O)—R³, with R³ being a substituted or unsubstituted C₆ heteroaryl, n of R²ₙ is 1 or 2, each R² independently from any other R² is a substituted or unsubstituted C₃-C₈ cycloalkyl, a substituted or unsubstituted C₁-C₆ alkyl, a substituted or unsubstituted C₁-C₆ alcohol, a substituted or unsubstituted C₆ heteroaryl, a halogen, in some instances —F, or —O—CH₂—R⁴, with R⁴ being a substituted or unsubstituted C₄ heteroaryl, Z¹, Z², Z³, Z⁴ and Z⁵ are independently of each other —C, —N or —O, A¹, A² and A³, are independently of each other —C, —N or —(C═O)—O—R⁷, with R⁷ being an alkyl, l of R⁵ₗ is 1 or 2, each R⁵ is independently from each other a C₁-C₄ alkinyl or a halogen, in some instances —Cl, k of R⁶ₖ is 1, 2, 3 or 4, each R⁶ is independently from each other a substituted or unsubstituted aryl, a substituted or unsubstituted heteroaryl, a substituted or unsubstituted C₁-C₃ alkyl, oxygen or hydrogen.

In certain embodiments a compound comprising the general formula (1a) is provided, wherein X¹, X², X³, X⁴ and X⁵ are independently from each other —C or —N, wherein at least two of X¹, X², X³, X⁴ and X⁵ are —N, Y¹ and Y² are independently from each other —C or —N, m of R¹ₘ, is 1, R¹ is a substituted or unsubstituted C₆ aryl or —(C═O)—R³, with R³ being a substituted or unsubstituted C₆ heteroaryl, n of R²ₙ is 1 or 2, each R² independently from any other R² is a substituted or unsubstituted C₃-C₈ cycloalkyl, a substituted or unsubstituted C₁-C₆ alkyl, a substituted or unsubstituted C₁-C₆ alcohol, a substituted or unsubstituted C₆ heteroaryl, a halogen, in some instances —F, or —O—CH₂—R⁴, with R⁴ being a substituted or unsubstituted C₄ heteroaryl.

In certain embodiments, a compound comprising the general formula (1b) is provided, wherein Z³, Z⁴ and Z⁵ are independently of each other —C or —N, A¹, A² and A³, are independently of each other —C, —N or —(C═O)—O—R⁷, with R⁷ being an alkyl, l of R⁵ₗ is 1 or 2, each R⁵ is independently from each other a C₁-C₄ alkinyl or a halogen, in some instances —Cl, k of R⁶ₖ is 1, 2, 3 or 4, in some instances 1 or 2, each R⁶ is independently from each other a substituted or unsubstituted aryl, a substituted or unsubstituted heteroaryl, a substituted or unsubstituted C₁-C₃ alkyl, oxygen or hydrogen.

In certain embodiments, a compound comprising the general formula (1c) is provided, wherein Z¹, Z², Z³, Z⁴ and Z⁵ are independently of each other —C, —N or —O, l of R⁵ is 1 or 2, each R⁵ is independently from each other a C₁-C₄ alkinyl or a halogen, in some instances —Cl, k of R⁶ₖ is 1, 2, 3 or 4, in some instances 1 or 2, each R⁶ is independently from each other a substituted or unsubstituted aryl, a substituted or unsubstituted heteroaryl, a substituted or unsubstituted C₁-C₃ alkyl, oxygen or hydrogen.

A GABA_{A} receptor modulator that can be used in the treatment of pain or associated conditions can be a compound of general formula (2), (3), (4), (5), (6) (7), or a pharmaceutically acceptable salt or solvate thereof,

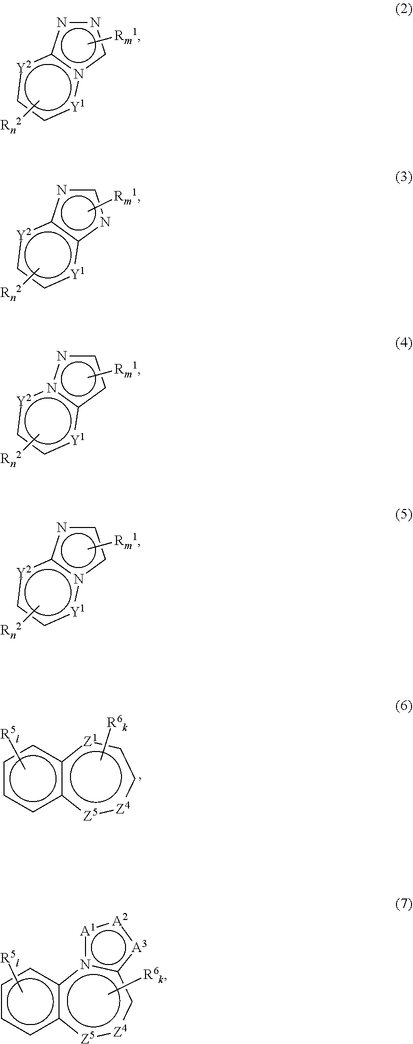

with Y¹, Y², Z¹, Z⁴, Z⁵, m of R¹ₘ, R¹, R³, n of R²ₙ, R², R⁴, l of R⁵ₗ, R⁵, k of R⁶ₖ, R⁶, A¹, A² and A³ having the same meaning as defined above.

In some embodiments, the GABA_{A} receptor modulator is a compound of formula (2), or a pharmaceutically acceptable salt or solvate thereof. In some embodiments, the GABA_{A} receptor modulator is a compound of formula (3), or a pharmaceutically acceptable salt or solvate thereof. In some embodiments, the GABA_{A} receptor modulator is a compound of formula (4), or a pharmaceutically acceptable salt or solvate thereof. In some embodiments, the GABA_{A} receptor modulator is a compound of formula (5), or a pharmaceutically acceptable salt or solvate thereof. In some embodiments, the GABA_{A} receptor modulator is a compound of formula (6), or a pharmaceutically acceptable salt or solvate thereof. In some embodiments, the GABA_{A} receptor modulator is a compound of formula (7), or a pharmaceutically acceptable salt or solvate thereof.

In certain embodiments, the compound comprises the general formula (2), (3), (4) or (5), with Y¹, Y², m of R¹ₘ, R³, n of R²ₙ, R² and R⁴ having the same meaning as defined above.

In certain embodiments, the compound comprises the general formula (6) with Z¹, Z⁴, Z⁵, l of R⁵ₗ, R⁵, k of R⁶ₖ and R⁶ having the same meaning as defined above.

In certain embodiments, the compound comprises the general formula (7) with Z⁴, Z⁵, l of R⁵ₗ, R⁵, k of R⁶ₖ, R⁶, A¹, A² and A³ having the same meaning as defined above.

A GABA_{A} receptor modulator that can be used in the treatment of pain or associated conditions can be a compound of general formula (2a), (3a), (4a), (5a), (5b), (6a), (6b), (7a), or a pharmaceutically acceptable salt or solvate thereof,

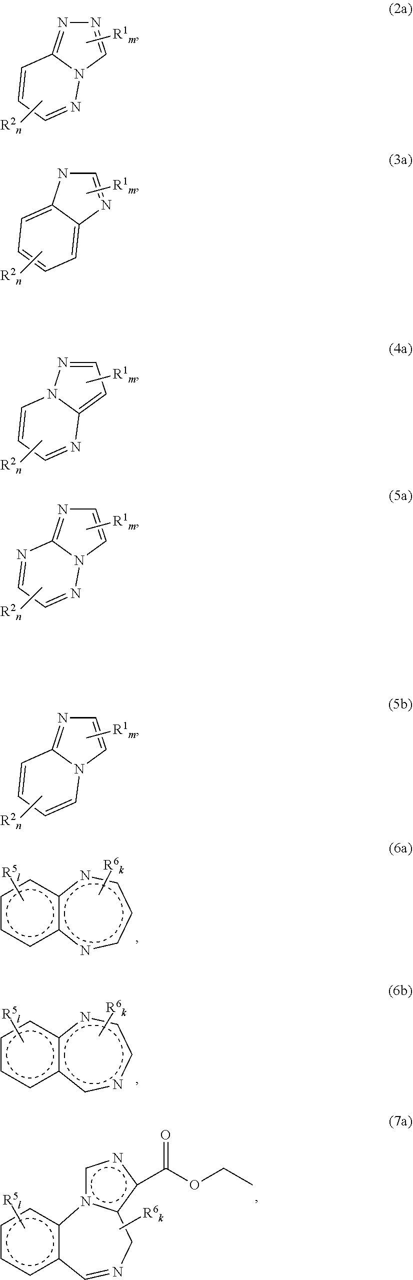

with m of R¹ₘ, R¹, R³, n of R²ₙ, R², R⁴, l of R⁵ₗ, R⁵, k of R⁶ₖ and R⁶ having the same meaning as defined above.

In some embodiments, the GABA_{A} receptor modulator is a compound of formula (2a), or a pharmaceutically acceptable salt or solvate thereof. In some embodiments, the GABA_{A} receptor modulator is a compound of formula (3a), or a pharmaceutically acceptable salt or solvate thereof. In some embodiments, the GABA_{A} receptor modulator is a compound of formula (4a), or a pharmaceutically acceptable salt or solvate thereof. In some embodiments, the GABA_{A} receptor modulator is a compound of formula (5a), or a pharmaceutically acceptable salt or solvate thereof. In some embodiments, the GABA_{A} receptor modulator is a compound of formula (5b), or a pharmaceutically acceptable salt or solvate thereof. In some embodiments, the GABA_{A} receptor modulator is a compound of formula (6a), or a pharmaceutically acceptable salt or solvate thereof. In some embodiments, the GABA_{A} receptor modulator is a compound of formula (6b), or a pharmaceutically acceptable salt or solvate thereof. In some embodiments, the GABA_{A} receptor modulator is a compound of formula (7a), or a pharmaceutically acceptable salt or solvate thereof.

In certain embodiments, the compound comprises the general formula (2a), (3a), (4a), (5a) or (5b) with m of R¹ₘ, R¹, R³, n of R²ₙ, R² and R⁴ having the same meaning as defined above.

In certain embodiments, the compound comprises the general formula (6a) or (6b) with m of l of R⁵ₗ, R⁵, k of R⁶ₖ and R⁶ having the same meaning as defined above.

In certain embodiments, the compound comprises the general formula (7a) with m of l of R⁵ₗ, R⁵, k of R⁶ₖ and R⁶ having the same meaning as defined above.

In certain embodiments, the compound comprises the general formula 1a, 1b, 1c, 2, 3, 4, 5, 6, 7, 2a, 3a, 4a, 5a, 5b, 6a, 6b or 7a, wherein m of R¹ is 1 and R¹ is: an unsubstituted phenyl, a substituted phenyl comprising at least one —F as a substituent, an unsubstituted biphenyl, a substituted biphenyl comprising at least one —CN as a substituent, in some instances on the phenyl moiety not connected to the parent moiety, or a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, or —(C═O)—R³, with R³ being pyridine, and l of R⁵ is 1 and R⁵ is Cl, Br, F, or a C₂ alkinyl.

In certain embodiments, the compound comprises the general formula 1a, 2, 3, 4, 5, 2a, 3a, 4a, 5a or 5b, wherein m of R¹ is 1 and R¹ is: an unsubstituted phenyl, a substituted phenyl comprising at least one —F as a substituent, an unsubstituted biphenyl, a substituted biphenyl comprising at least one —CN as a substituent, in some instances on the phenyl moiety not connected to the parent moiety, or a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, or —(C═O)—R³, with R³ being pyridine.

In certain embodiments, the compound comprises the general formula 1b, 7, or 7a, wherein l of R⁵ is 1 and R⁵ is Cl, Br, F, or a C₂ alkinyl.

In certain embodiments, the compound comprises the general formula 1c, 6, 6a or 6b, wherein l of R⁵ is 1 and R⁵ is Cl, Br, F, or a C₂ alkinyl.

In certain embodiments, the compound comprises the general formula 1a, 1b, 1c, 2, 3, 4, 5, 6, 7, 2a, 3a, 4a, 5s, 5b, 6a, 6b or 7a, wherein n of R² is 1 or 2 and in case of n being 2, each R² independently from each other is an unsubstituted C₃-C₈ cycloalkyl, in some instances a C₄-cycloalkyl, an unsubstituted C₁-C₆ alkyl, in some instances tert-butyl, or —O—CH₂—R⁴, with R⁴ being a substituted or unsubstituted C₄ heteroaryl, in some instances R⁴ being a substituted or unsubstituted triazole, an unsubstituted C₁-C₆ alcohol, in some instances a C₄ alcohol, a halogen, in some instances —F, in case of n being 1, R² is an unsubstituted C₁-C₆ alcohol, in some instances a C₄ alcohol, an unsubstituted C₆ heteroaryl, in some instances pyridine, and k of R⁶ is 1 or 4 and in case of k being 1, R⁶ is a substituted or unsubstituted aryl, a substituted or unsubstituted heteroaryl, in case of k being 4, each R⁶ independently from each other is a substituted or unsubstituted aryl, a substituted or unsubstituted C₁-C₃ alkyl, oxygen, or hydrogen.

In certain embodiments, the compound comprises the general formula 1a, 1b, 1c, 2, 3, 4, 5, 6, 7, 2a, 3a, 4a, 5a, 5b, 6a, 6b or 7a, wherein n of R² is 1 or 2 and in case of n being 2, each R² independently from each other is an unsubstituted C₃-C₈ cycloalkyl, in some instances a C₄-cycloalkyl, an unsubstituted C₁-C₆ alkyl, in some instances tert-butyl, or —O—CH₂—R⁴, with R⁴ being a substituted or unsubstituted C₄ heteroaryl, in some instances R⁴ being a substituted or unsubstituted triazole, an unsubstituted C₁-C₆ alcohol, in some instances a C₄ alcohol, a halogen, in some instances —F, in case of n being 1, R² is an unsubstituted C₁-C₆ alcohol, in some instances a C₄ alcohol, an unsubstituted C₆ heteroaryl, in some instances pyridine, and k of R⁶ is 1 or 4 and in case of k being 1, R⁶ is a substituted or unsubstituted aryl, a substituted or unsubstituted heteroaryl, in case of k being 4, each R⁶ independently from each other is a substituted or unsubstituted aryl, a substituted or unsubstituted C₁-C₃ alkyl, oxygen, or hydrogen.

In certain embodiments, the compound comprises the general formula 1a, 2, 3, 4, 5, 2a, 3a, 4a, 5a or 5b, wherein n of R² is 1 or 2 and in case of n being 2, each R² independently from each other is an unsubstituted C₃-C₈ cycloalkyl, in some instances a C₄-cycloalkyl, an unsubstituted C₁-C₆ alkyl, in some instances tert-butyl, or —O—CH₂—R⁴, with R⁴ being a substituted or unsubstituted C₄ heteroaryl, in some instances R⁴ being a substituted or unsubstituted triazole, an unsubstituted C₁-C₆ alcohol, in some instances a C₄ alcohol, a halogen, in some instances —F, in case of n being 1, R² is an unsubstituted C₁-C₆ alcohol, in some instances a C₄ alcohol, an unsubstituted C₆ heteroaryl, in some instances pyridine.

In certain embodiments, the compound comprises the general formula 1b, 7, or 7a, wherein k of R⁶ is 1 or 4 and in case of k being 1, R⁶ is a substituted or unsubstituted aryl, a substituted or unsubstituted heteroaryl, in case of k being 4, each R⁶ independently from each other is a substituted or unsubstituted aryl, a substituted or unsubstituted C₁-C₃ alkyl, oxygen, or hydrogen.

In certain embodiments, the compound comprises the general formula 1c, 6, 6a or 6b wherein k of R⁶ is 1 or 4 and in case of k being 1, R⁶ is a substituted or unsubstituted aryl, a substituted or unsubstituted heteroaryl, in case of k being 4, each R⁶ independently from each other is a substituted or unsubstituted aryl, a substituted or unsubstituted C₁-C₃ alkyl, oxygen, or hydrogen.

In certain embodiments, the compound comprises the general formula 1a, 1b, 1c, 2, 3, 4, 5, 6, 7, 2a, 3a, 4a, 5s, 5b, 6a, 6b or 7a, wherein n of R² is 2 and one R² is an unsubstituted C₃-C₈ cycloalkyl, in some instances a C₄-cycloalkyl, or an unsubstituted C₁-C₆ alkyl, in some instances tert-butyl, and the other R² is —O—CH₂—R⁴, with R⁴ being a substituted or unsubstituted C₄ heteroaryl, in some instances a substituted or unsubstituted triazole, or one R² is an unsubstituted C₁-C₆ alcohol, in some instances a C₄ alcohol, and the other R² is a halogen, in some instances —F, and k of R⁶ is 4 and two R⁶ are oxygen, the other R⁶ are independently from each other a substituted or unsubstituted aryl, a substituted or unsubstituted C₁-C₃ alkyl, or hydrogen.

In certain embodiments, the compound comprises the general formula 1a, 2, 3, 4, 5, 2a, 3a, 4a, 5s or 5b, wherein n of R² is 2 and one R² is an unsubstituted C₃-C₈ cycloalkyl, in some instances a C₄-cycloalkyl, or an unsubstituted C₁-C₆ alkyl, in some instances tert-butyl, and the other R² is O—CH₂—R⁴, with R⁴ being a substituted or unsubstituted C₄ heteroaryl, in some instances a substituted or unsubstituted triazole, or one R² is an unsubstituted C₁-C₆ alcohol, in some instances a C₄ alcohol, and the other R² is a halogen, in some instances —F.

In certain embodiments, the compound comprises the general formula 1b, 7, or 7a, wherein k of R⁶ is 4 and two R⁶ are oxygen, the other R⁶ are independently from each other a substituted or unsubstituted aryl, a substituted or unsubstituted C₁-C₃ alkyl, or hydrogen.

In certain embodiments, the compound comprises the general formula 1c, 6, 6a or 6b, wherein k of R⁶ is 4 and two R⁶ are oxygen, the other R⁶ are independently from each other a substituted or unsubstituted aryl, a substituted or unsubstituted C₁-C₃ alkyl, or hydrogen.

A GABA_{A} receptor modulator that can be used in the treatment of pain or an associated condition can be a compound of general formula (2a′), (3a′), (4a′), (5a′), (5b′), (6a′), (6b′), (7a′), or a pharmaceutically acceptable salt or solvate thereof,

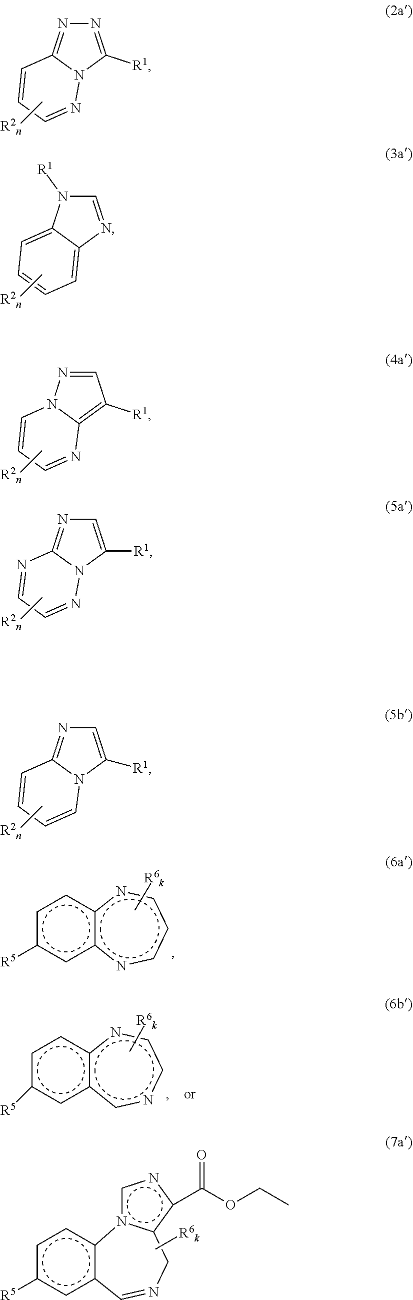

with R¹, R³ n of R²ₙ, R², R⁴, R⁵, k of R⁶ₖ and R⁶ having the same meaning as defined above.

In some embodiments, the GABA_{A} receptor modulator is a compound of formula (2a′), or a pharmaceutically acceptable salt or solvate thereof. In some embodiments, the GABA_{A} receptor modulator is a compound of formula (3a′), or a pharmaceutically acceptable salt or solvate thereof. In some embodiments, the GABA_{A} receptor modulator is a compound of formula (4a′), or a pharmaceutically acceptable salt or solvate thereof. In some embodiments, the GABA_{A} receptor modulator is a compound of formula (5a′), or a pharmaceutically acceptable salt or solvate thereof. In some embodiments, the GABA_{A} receptor modulator is a compound of formula (5b′), or a pharmaceutically acceptable salt or solvate thereof. In some embodiments, the GABA_{A} receptor modulator is a compound of formula (6a′), or a pharmaceutically acceptable salt or solvate thereof. In some embodiments, the GABA_{A} receptor modulator is a compound of formula (6b′), or a pharmaceutically acceptable salt or solvate thereof. In some embodiments, the GABA_{A} receptor modulator is a compound of formula (7a′), or a pharmaceutically acceptable salt or solvate thereof.

In certain embodiments, the compound comprises the general formula (2a′), (3a′), (4a′), (5a′) or (5b′) with R¹, R³, n of R²ₙ, R² and R⁴ having the same meaning as defined above.

In certain embodiments, the compound comprises the general formula (6a′) or (6b′) with k of R⁶ₖ and R⁶ having the same meaning as defined above.

In certain embodiments, the compound comprises the general formula (7a′) with k of R⁶ₖ and R⁶ having the same meaning as defined above.

In certain embodiments, the compound comprises the general formula (2a′), (3a′), (4a′), (5a′), (5b′), (6a′), (6b′) or (7a′), wherein R¹ is in case of formula (2a′) a substituted or unsubstituted C₆ aryl, in some instances an unsubstituted phenyl, a substituted phenyl comprising at least one —F as a substituent, in case of formula (3a′), (5a′) or (5b′), a substituted or unsubstituted biphenyl, in some instances an unsubstituted biphenyl, a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, or in case of formula (4a′), —(C═O)—R³, with R³ being a substituted or unsubstituted C₆ heteroaryl, in some instances with R³ being pyridine, R⁵ is in case of formula (6a′) or (6b′), Cl, Br or F, in case of formula (7a′) C₂ alkinyl, wherein R²ₙ and R⁶ₖ have the same meaning as defined above.

In certain embodiments, the compound comprises the general formula (2a′), (3a′), (4a′), (5a′) or (5b′) wherein R¹ is in case of formula (2a′) a substituted or unsubstituted C₆ aryl, in some instances an unsubstituted phenyl, a substituted phenyl comprising at least one —F as a substituent, in case of formula (3a′), (5a′) or (5b′), a substituted or unsubstituted biphenyl, in some instances an unsubstituted biphenyl, a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, or in case of formula (4a′), —(C═O)—R³, with R³ being a substituted or unsubstituted C₆ heteroaryl, in some instances with R³ being pyridine, wherein R²ₙ has the same meaning as defined above.

In certain embodiments, the compound comprises the general formula (6a′) or (6b′), wherein R⁵ is Cl, Br or F, wherein R⁶ₖ has the same meaning as defined above.

In certain embodiments, the compound comprises the general formula (7a′), wherein R⁵ is C² alkinyl, wherein R⁶ₖ has the same meaning as defined above.

A GABA_{A} receptor modulator that can be used in the treatment of pain or associated conditions can be a compound of general formula (2a″), (3a″), (4a″), (5a″), (5b″), (6a″), (7a″), or a pharmaceutically acceptable salt or solvate thereof,

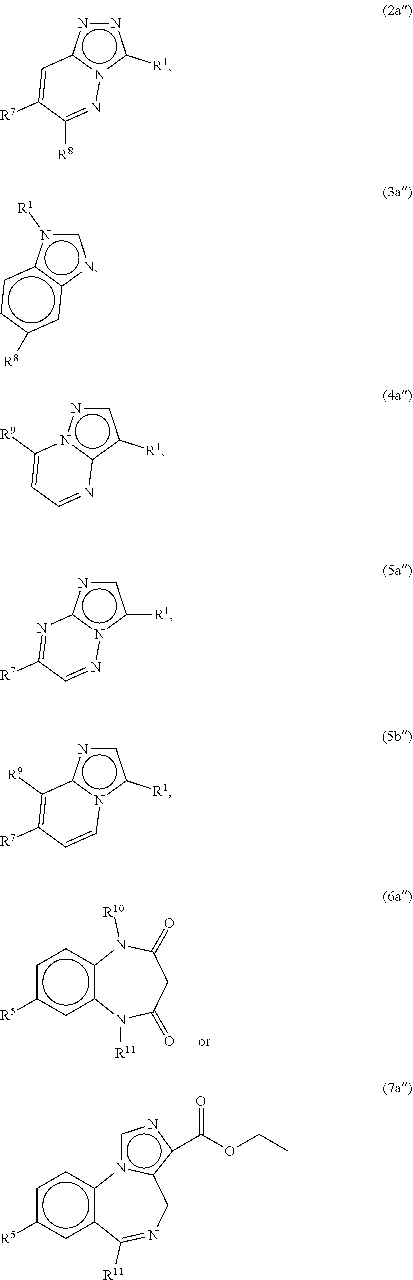

with R⁷ being an unsubstituted C₁-C₆ alkyl, in some instances tert-butyl, an unsubstituted C₃-C₈ cycloalkyl, in some instances a C₄-cycloalkyl, an unsubstituted C₁-C₆ alcohol, in some instances a C₄ alcohol, in some instances R⁷ being in case of formula (2a″) an unsubstituted C₁-C₆ alkyl, in some instances tert-butyl, or an unsubstituted C₃-C₈ cycloalkyl, in some instances a C₄-cycloalkyl, or R⁷ being in case of formula (5a″) or (5b″) an unsubstituted C₁-C₆ alcohol, in some instances a C₄ alcohol, R⁸ being —O—CH 2-R⁴, with R⁴ being a substituted or unsubstituted C₄ heteroaryl, in some instances a substituted or unsubstituted triazole, or an unsubstituted C₁-C₆ alcohol, in some instances a C₄ alcohol, in some instances R⁸ being in case of formula (2a″) —O—CH₂—R⁴, with R⁴ being a substituted or unsubstituted C₄ heteroaryl, in some instances a substituted or unsubstituted triazole, or R⁸ being in case of formula (3a″) an unsubstituted C₁-C₆ alcohol, in some instances a C₄ alcohol, R⁹ being an unsubstituted C₆ heteroaryl, in some instances pyridine, or a halogen, in some instances —F, R⁹ being in case of formula (4a″) an unsubstituted C₆ heteroaryl, in some instances pyridine, or R⁹ being in case of formula (5b″) a halogen, in some instances —F, R¹⁰ being a C₁-C₃ alkyl or hydrogen, R¹¹ being a substituted or unsubstituted aryl or a substituted or unsubstituted heteroaryl, in some instances R¹¹ being in case of formula (6a″) an substituted or unsubstituted aryl, in some instances phenyl R¹¹ being in case of formula (7a″) a substituted or unsubstituted heteroaryl, in some instances pyridine, a substituted or unsubstituted aryl, in some instances phenyl, R⁵ being in case of formula (6a″), Cl, Br or F, in case of formula (7a″) C₂ alkinyl.

In some embodiments, the GABA_{A} receptor modulator is a compound of formula (2a″), or a pharmaceutically acceptable salt or solvate thereof. In some embodiments, the GABA_{A} receptor modulator is a compound of formula (3a″), or a pharmaceutically acceptable salt or solvate thereof. In some embodiments, the GABA_{A} receptor modulator is a compound of formula (4a″), or a pharmaceutically acceptable salt or solvate thereof. In some embodiments, the GABA_{A} receptor modulator is a compound of formula (5a″), or a pharmaceutically acceptable salt or solvate thereof. In some embodiments, the GABA_{A} receptor modulator is a compound of formula (5b″), or a pharmaceutically acceptable salt or solvate thereof. In some embodiments, the GABA_{A} receptor modulator is a compound of formula (6a″), or a pharmaceutically acceptable salt or solvate thereof. In some embodiments, the GABA_{A} receptor modulator is a compound of formula (7a″), or a pharmaceutically acceptable salt or solvate thereof.

In certain embodiments, the compound comprises the general formula (2a″), (3a″), (4a″), (5a″) or (5b″) with R⁷ being an unsubstituted C₁-C₆ alkyl, in some instances tert-butyl, an unsubstituted C₃-C₈ cycloalkyl, in some instances a C₄-cycloalkyl, an unsubstituted C₁-C₆ alcohol, in some instances a C₄ alcohol, in some instances R⁷ being in case of formula (2a″) an unsubstituted C₁-C₆-alkyl, in some instances tert-butyl, or an unsubstituted C₃-C₈ cycloalkyl, in some instances a C₄-cycloalkyl, or R⁷ being in case of formula (5a″) or (5b″) an unsubstituted C₁-C₆ alcohol, in some instances a C₄ alcohol, R⁸ being —O—CH₂—R⁴, with R⁴ being a substituted or unsubstituted C₄ heteroaryl, in some instances a substituted or unsubstituted triazole, or an unsubstituted C₁-C₆ alcohol, in some instances a C₄ alcohol, in some instances R⁸ being in case of formula (2a″) —O—CH₂—R⁴, with R⁴ being a substituted or unsubstituted C₄ heteroaryl, in some instances a substituted or unsubstituted triazole, or R⁸ being in case of formula (3a″) an unsubstituted C₁-C₆ alcohol, in some instances a C₄ alcohol, R⁹ being an unsubstituted C₆ heteroaryl, in some instances pyridine, or a halogen, in some instances —F, R⁹ being in case of formula (4a″) an unsubstituted C₆ heteroaryl, in some instances pyridine, or R⁹ being in case of formula (5b″) a halogen, in some instances —F.

In certain embodiments, the compound for treatment of pain or associated conditions comprises the general formula (6a″) with R¹⁰ being a C₁-C₃ alkyl or hydrogen, R¹¹ being a substituted or unsubstituted aryl or a substituted or unsubstituted heteroaryl, in some instances R¹¹ being in case of formula (6a″) an substituted or unsubstituted aryl, in some instances phenyl R¹¹ being in case of formula (7a″) a substituted or unsubstituted heteroaryl, in some instances pyridine, R⁵ being Cl, Br or F.

In certain embodiments, the compound comprises the general formula (7a″) with R¹¹ being a substituted or unsubstituted aryl or a substituted or unsubstituted heteroaryl, in some instances R¹¹ being in case of formula (6a″) an substituted or unsubstituted aryl, in some instances phenyl R¹¹ being in case of formula (7a″) a substituted or unsubstituted heteroaryl, in some instances pyridine, R⁵ being C₂ alkinyl.

In certain embodiments, the compound comprises the general formula (2a″), (3a″), (4a″), (5a″) or (5b″) with in case of formula (2a″) R¹ being or an unsubstituted phenyl, a substituted phenyl comprising at least one —F as a substituent, R⁷ being an unsubstituted C₁-C₆ alkyl, in some instances tert-butyl, or an unsubstituted C₃-C₈ cycloalkyl, in some instances a C₄-cycloalkyl, and R⁸ being —O—CH₂—R⁴, with R⁴ being a substituted or unsubstituted C₄ heteroaryl, in some instances a substituted or unsubstituted triazole, in case of formula (3a″) R¹ being an unsubstituted biphenyl, a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, being an unsubstituted C₁-C₆ alcohol, in some instances a C₄ alcohol, in case of formula (4a″) R¹ being —(C═O)—R³, with R³ being an unsubstituted C₆ heteroaryl, in some instances R³ being pyridine, and R⁹ being an unsubstituted C₆ heteroaryl, in some instances pyridine, in case of formula (5a″) R¹ being an unsubstituted biphenyl, a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, R⁷ being an unsubstituted C₁-C₆ alcohol, in some instances a C₄ alcohol, in case of formula (5a″) R¹ being an unsubstituted biphenyl, a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, R⁷ being an unsubstituted C₁-C₆ alcohol, in some instances a C₄ alcohol, R⁹ being a halogen, in some instances —F.

A GABA_{A} receptor modulator that can be used in the treatment of pain or associated conditions can be a compound of general formula (8), or a pharmaceutically acceptable salt or solvate thereof,

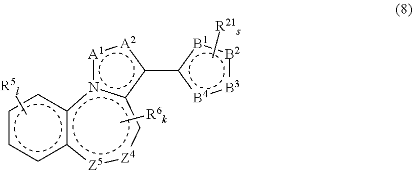

where
—Z⁴ and Z⁵ are independently of each other —C, —N, —S or —O,
A¹ and A² are independently of each other —C, —N or —(C═O)—O—R⁷, with R⁷ being an alkyl,
B¹, B², B³, and B⁴ are independently of each other —C, —N, or —O,
l of R⁵ₗ is 1 or 2,
each R⁵ is independently from each other a C₁-C₄ alkinyl or a halogen, in some instances —Cl,
k of R⁶ₖ is 1, 2, 3 or 4, in some instances 1 or 2,
each R⁶ is independently from each other a substituted or unsubstituted aryl, a substituted or unsubstituted heteroaryl, a substituted or unsubstituted C₁-C₃ alkyl, oxygen or hydrogen,
s of R²¹ₛ is 1, 2, 3 or 4, or
each R²¹ is independently hydrogen or C₁-C₆ alkyl.

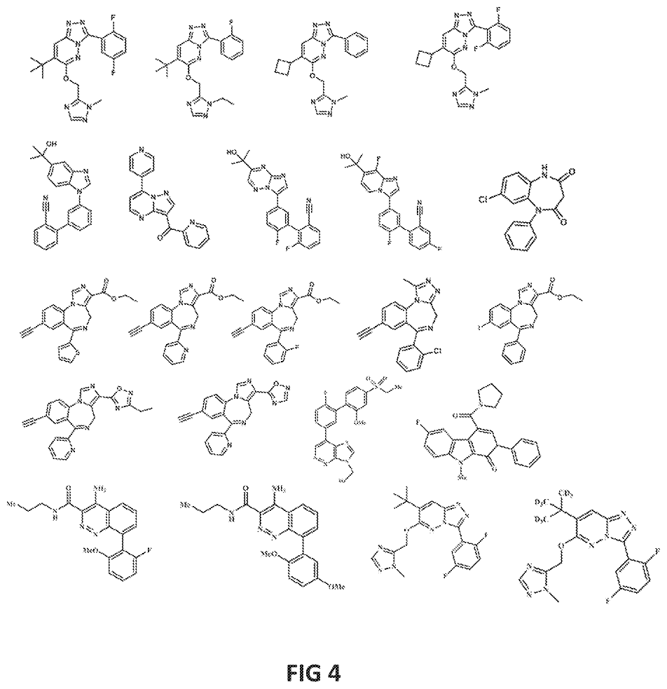

In certain embodiments, the compound is selected from the compounds depicted in FIG. 4, such as L-838417, TPA023 (MK-0777), TPA123, MRK-409(MK-0343), NS11394, Ocinaplon(DOV-273547), Compound 1, TP003, N-Desmethylclobazam 1, 2, 3, 4 and 5, Hz-166, MP-III-080, KRM-II-81, and pharmaceutically acceptable salts or solvates thereof.

According to a first subaspect of the invention, a GABA_{A} receptor modulator that can be used in the treatment of fibromyalgia and conditions associated with fibromyalgia. In some cases, a compound can be a compound of general formula (1a), general formula (1b), general formula (1c), or a pharmaceutically acceptable salt or solvate thereof,

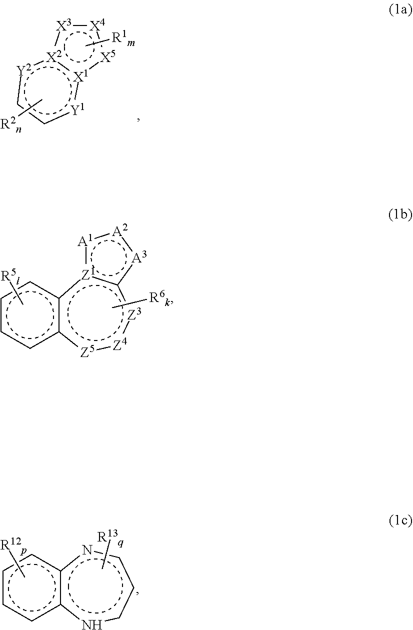

in some instances formula (1a) and (1b), more particular

(1a), wherein
X¹, X², X³, X⁴ and X⁵ are independently from each other —C, —N, —S or —O wherein at least two of X¹, X², X³, X and X⁵ are —N, and Y² are independently from each other —C or —N,
m of R¹ₘ is 1,
R¹ is an unsubstituted phenyl, a phenyl substituted with C₁-C₄-alkyl, F, Cl, Br, I, —CN, a substituted or unsubstituted biphenyl or —(C═O)—R³, with R³ being a substituted or unsubstituted aryl or 5- to 6-membered heteroaryl, in some instances a C₆ aryl or 6-membered heteroaryl, in some instances a 6-membered heteroaryl,
n of R²ₙ is 1 or 2,
each R² independently from any other R² is a substituted or unsubstituted C₃-C₈ cycloalkyl, a substituted or unsubstituted C₁-C₆ alkyl, a substituted or unsubstituted C₁-C₆ alcohol, a substituted or unsubstituted 6-membered heteroaryl, a halogen, in some instances —F, or —O—CH 2-R⁴, with R⁴ being a substituted or unsubstituted 5- or 6-membered heteroaryl, in some instances a 5-membered heteroaryl,
Z¹, Z³, Z⁴ and Z⁵ are independently of each other —C, —N, —S or —O, in some instances Z¹ is —C or —N and Z³ and Z⁴ are —C, —N, —S or —O,
A¹, and A² and A³, are independently of each other —C, —N, —(C═O)—O—R⁷, or

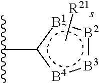

where R⁷ is alkyl,
B¹, B², B³, and B⁴ are independently of each other —C, —N, or —O,
s of R²¹ₛ is 1, 2, 3 or 4, and
each R²¹ is independently hydrogen or C₁-C₆ alkyl,
l of R⁵ₗ is 1 or 2
each R⁵ is independently from each other a C₁-C₄ alkinyl or a halogen,
k of R⁶ₖ is 1, 2, 3 or 4, in some instances 1 or 2, each R⁶ is independently from each other a substituted or unsubstituted aryl, a substituted or unsubstituted heteroaryl, a substituted or unsubstituted C₁-C₃ alkyl, oxygen or hydrogen, in some instances a substituted or unsubstituted aryl, a substituted or unsubstituted heteroaryl, a substituted or unsubstituted C₁-C₃ alkyl, in some instances a substituted or unsubstituted aryl, a substituted or unsubstituted heteroaryl,
p of R¹² is 1 or 2, in some instances 1,
R¹² is independently from each other a substituted or unsubstituted C₁-C₄-alkyl, I, Br, Cl or F,
q of R¹³ is 1, 2, 3 or 4,
R¹³ is independently from each other a substituted or unsubstituted aryl, a substituted or unsubstituted heteroaryl, a substituted or unsubstituted _{C1-C3} alkyl, oxygen or hydrogen.

In certain embodiments, a GABA_{A} receptor modulator that can be used in the treatment of fibromyalgia and associated conditions can be a compound of general formula (1a), general formula (1b) or general formula (1c), in some instances formula (1a) and (1b), more particular (1a), or a pharmaceutically acceptable salt or solvate thereof, wherein
X¹, X², X³, X⁴ and X⁵ are independently from each other —C or —N, wherein at least two of X¹, X², X³, X⁴ and X⁵ are —N,
Y¹ and Y² are independently from each other —C or —N,
m of R¹ₘ is 1,
R¹ is an unsubstituted phenyl, a phenyl substituted with C₁-C₄-alkyl, F, Cl, Br, I, —CN, a substituted or unsubstituted biphenyl or —(C═O)—R³, with R³ being a substituted or unsubstituted aryl or 5- to 6-membered heteroaryl, in some instances a C₆ aryl or 6-membered heteroaryl, in some instances a 6-membered heteroaryl,
n of R²ₙ is 1 or 2,
each R₂ independently from any other R² is a substituted or unsubstituted C₃-C₈ cycloalkyl, a substituted or unsubstituted C₁-C₆ alkyl, a substituted or unsubstituted C₁-C₆ alcohol, a substituted or unsubstituted 6-membered heteroaryl, a halogen, in some instances —F, or —O—CH₂—R⁴, with R⁴ being a substituted or unsubstituted 5- or 6-membered heteroaryl, in some instances a 5-membered heteroaryl,
Z¹, Z³, Z⁴ and Z⁵ are independently of each other —C, or —N,
A¹ and A² are independently of each other —C or —N and A³ is —(C═O)—O—R^{7′}, with R^{7′} being a C₁-C₄-alkyl, in some instances a C₁-C₂-alkyl, in some instances a C₂-alkyl,
l of R⁵ₗ is 1 or 2
each R⁵ is independently from each other a C₁-C₄ alkinyl or a halogen,
k of R⁶ₖ is 1, 2, 3 or 4, in some instances 1 or 2
each R⁶ is independently from each other a substituted or unsubstituted aryl, a substituted or unsubstituted heteroaryl, a substituted or unsubstituted C₁-C₃ alkyl, oxygen or hydrogen, in some instances a substituted or unsubstituted aryl, a substituted or unsubstituted heteroaryl, a substituted or unsubstituted C₁-C₃ alkyl, in some instances a substituted or unsubstituted aryl, a substituted or unsubstituted heteroaryl,
p of R¹² is 1,
R¹² is a substituted or unsubstituted C₁-C₄-alkyl, I, Br, Cl or F,
q of R¹³ is 1, 2, 3 or 4,
R¹³ is independently from each other a substituted or unsubstituted aryl, a substituted or unsubstituted heteroaryl, a substituted or unsubstituted C₁-C₃ alkyl, oxygen or hydrogen.

In certain embodiments, a GABA_{A} receptor modulator that can be used in the treatment of fibromyalgia and associated conditions can be a compound of general formula (2), (3), (4), (5), (1c) or (7), in some instances (2), (3), (4), (5) or (7), in some instances (2), (3), (4) or (5), or a pharmaceutically acceptable salt or solvate thereof,

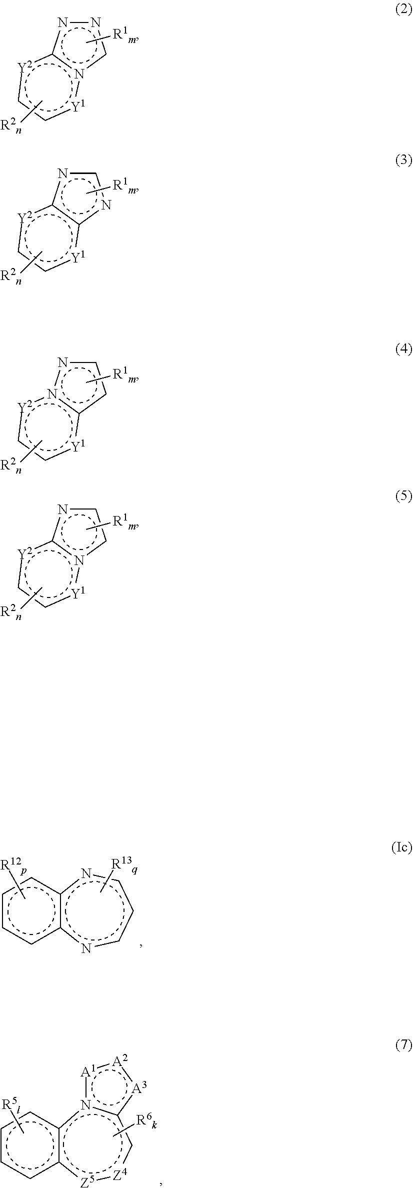

with Y¹, Y², Z⁴, Z⁵, m or R¹ₘ, R¹, R³, n of R²ₙ, R², R⁴, p of R¹², q of R¹³, R¹³, l of R⁵ₗ, R⁵, k of R⁶ₖ, R⁶, A¹, A² and A³ having the same meaning as defined above.

In certain embodiments, the compound for treatment of fibromyalgia and associated conditions comprises the general formula (2a), (3a), (4a), (5a), (5b), (1c) or (7a), in some instances (2a) (3a), (4a) (5a), (5b) or (7a), in some instances (2a), (3a) (4a), (5a) or (5b), or a pharmaceutically acceptable salt or solvate thereof,

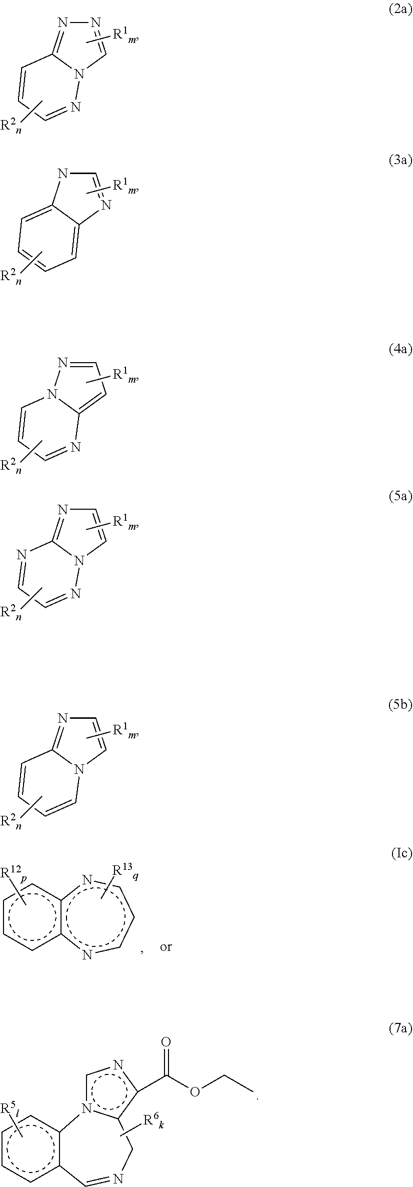

with m of R¹ₘ, R³, n of R²ₙ, R², R⁴, p of R¹², R¹², of R¹³, R¹³, l of R⁵ₗ, R⁵, k of R⁶ₖ and R⁶ having the same meaning as defined above.

In certain embodiments, m of R¹ is 1 and R¹ is: an unsubstituted phenyl, a substituted phenyl comprising C₁-C₄-alkyl, F, Cl, Br, I, —CN as substituents, wherein in some instances said substituted phenyl comprises at least one —F as a substituent, an unsubstituted biphenyl, a substituted biphenyl comprising at least one —CN as a substituent, in some instances on the phenyl moiety not connected to the parent moiety, or a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, or —(C═O)—R³, with R³ being pyridine.

In certain embodiments, m of R¹ is 1 and R¹ is a substituted phenyl comprising C₁-C₄-alkyl, F, Cl, Br, I, —CN as substituents, wherein in some instances said substituted phenyl comprises at least one —F as a substituent. The substituted phenyl is a phenyl moiety according to formula (9),

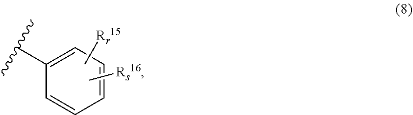

wherein r is 1, 2, 3, 4 or 5 and R¹⁵ is F and s is 0, 1, 2, 3 or 4 and R¹⁶ is C₁-C₄-alkyl, Cl, Br, I, —CN, wherein the sum of r and s does not exceed 5.

In certain embodiments, m of R¹ is 1 and R¹ is a substituted phenyl according to formula (9), wherein r is 1 or 2 and R¹⁵ is F and wherein s is 0, 1, 2, 3 or 4 (in case of r being 2, s is 0, 1, 2 or 3) and R¹⁶ is C₁-C₄-alkyl, Cl, Br, I, —CN.

In certain embodiments, m of R¹ is 1 and R¹ is a substituted phenyl according to formula (9), wherein r is 1 or 2 and R¹⁵ is F and wherein s is 0.

In certain embodiments, m of R¹ is 1 and R¹ is a substituted biphenyl comprising at least one —CN as a substituent, in some instances on the phenyl moiety not connected to the parent moiety. The substituted biphenyl can be a biphenyl moiety according to formula (10),

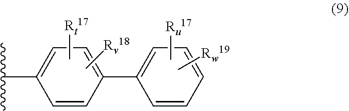

wherein t is 0, 1, 2, 3 or 4, u is 0, 1, 2, 3, 4 or 5 and R¹⁷ is —CN, wherein at least t and/or u is equal or greater 1, v is 0, 1, 2, 3 or 4 and R¹⁸ is C₁-C₄-alkyl, Cl, Br, I or —CN, and w is 0, 1, 2, 3, 4 or 5 and R¹⁹ is C₁-C₄-alkyl, Cl, Br, I or —CN, wherein the sum of t, u, v and w does not exceed 9.

In certain embodiments, m of R¹ is 1 and R¹ is a substituted biphenyl according to formula (10), wherein v and w are 0 or 1, R¹⁸ and R¹⁹ are C₁-C₄-alkyl, Cl, Br, I or —CN, t and/or u are 1 and R¹⁷ is —CN.

In certain embodiments, m of R¹ is 1 and R¹ is a substituted biphenyl according to formula (10), wherein v and w are 0, t and/or u are 1 and R¹⁷ is —CN.

In certain embodiments, m of R¹ is 1 and R¹ is a substituted biphenyl according to formula (10), wherein v, w and t are 0, u is 1 and R¹⁷ is —CN.

In certain embodiment, m of R¹ is 1 and R¹ is a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one substituent. This substituted biphenyl can be a biphenyl moiety according to formula (11),

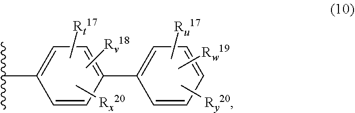

wherein t is 0, 1, 2, 3 or 4, u is 0, 1, 2, 3, 4 or 5 and R¹⁷ is —CN, wherein at least t and/or u is equal or greater 1, v is 0, 1, 2, 3 or 4 and R¹⁸ is C₁-C₄-alkyl, Cl, Br, I or —CN, w is 0, 1, 2, 3, 4 or 5 and R¹⁹ is C₁-C₄-alkyl, Cl, Br, I or —CN, x is 0, 1, 2, 3, or 4, y is 0, 1, 2, 3, 4 or 5 and R²⁰ is F, wherein at least x and/or y is equal or greater 1 wherein the sum of t, u, v, w, x and y does not exceed 9.

In certain embodiments, m of R¹ is 1 and R¹ is a substituted biphenyl according to formula (11), wherein v and w are 0, t and/or u are 1, R¹⁷ is —CN, x and/or y are 1 and R²⁰ is —F.

In certain embodiments, m of R¹ is 1 and R¹ is a substituted biphenyl according to formula (11), wherein v, w and t are 0, u is 1, R¹⁷ is —CN, x and/or y are 1 and R²⁰ is —F.

In certain embodiments, m of R¹ is 1 and R¹ is a substituted biphenyl according to formula (11), wherein v, w and t are 0, u is 1, R¹⁷ is —CN, x and y are 1 and R²⁰ is —F.

in certain embodiments, n of R² is 1 or 2 and R² is an unsubstituted C₃-C₈ cycloalkyl, in some instances a C₄-cycloalkyl, an unsubstituted C₁-C₆ alkyl, in some instances tert-butyl, or —O—CH₂—R⁴, with R⁴ being a substituted or unsubstituted 5-membered heteroaryl, in some instances R⁴ being a substituted or unsubstituted triazole, an unsubstituted C₁-C₆ alcohol, in some instances a C₃ alcohol, in some instances isopropanol, a halogen, in some instances —F, an unsubstituted 6-membered heteroaryl, in some instances pyridine.

In certain embodiments, n of R² is 2 and one R² is an unsubstituted C₃-C₈ cycloalkyl, in some instances a C₄-cycloalkyl, or an unsubstituted C₁-C₆ alkyl, in some instances tert-butyl, and the other R² is O—CH₂—R⁴, with R⁴ being a substituted or unsubstituted 5-membered heteroaryl, in some instances a substituted or unsubstituted triazole.

In certain embodiments, n of R² is 2 and one R² is an unsubstituted C₁-C₆ alcohol, in some instances a C₃ alcohol, in some instances isopropanol, and the other R² is a halogen, in some instances —F.

In certain embodiments, n of R² is 1 and R² is an unsubstituted C₁-C₆ alcohol or an unsubstituted 6-membered heteroaryl.

In certain embodiments, n of R² is 1 and R² is a C₃ alcohol or pyridine.

In certain embodiments, n of R² is 1 and R² is a C₃ alcohol.

In certain embodiments, n of R² is 1 and R² is isopropanol.

In certain embodiments, l of R⁵ is 1 and R⁵ is I, Cl, Br, F, or a C₂ alkinyl.

In certain embodiments, l of R⁵ is 1 and R⁵ is I or a C₂ alkinyl.

In certain embodiments, k of R⁶ is 1 and R⁶ is a substituted or unsubstituted aryl, a substituted or unsubstituted heteroaryl, a substituted or unsubstituted C₁-C₃ alkyl.

In certain embodiments, k of R⁶ is 1 and R⁶ is a substituted or unsubstituted C₆ aryl, a 5- to 6-membered substituted or unsubstituted heteroaryl.

In certain embodiments, k of R⁶ is 1 and R⁶ is a phenyl, a phenyl substituted with F or Cl, thiophen or pyridine.

In certain embodiments, a GABA_{A} receptor modulator that can be used in the treatment of fibromyalgia and associated conditions can be a compound of general formula (2a′), (3a′), (4a′), (5a′), (5b′), (VI) or (7a′), in some instances (2a′), (3a′), (4a′), (5a′), (5b′) or (7a′), even in some instances (2a′), (3a′), (4a′), (5a′) or (5b′), or a pharmaceutically acceptable salt or solvate thereof,

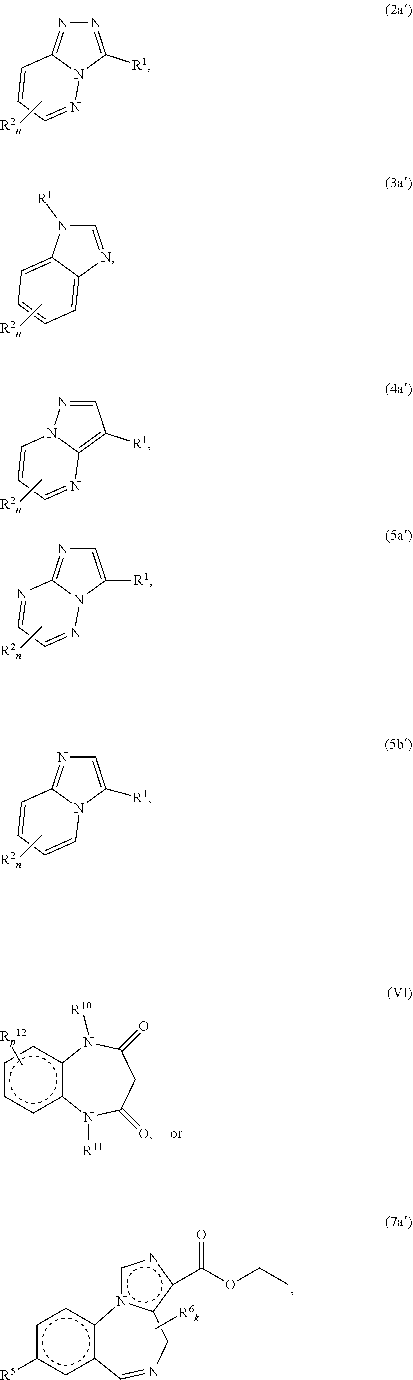

wherein
R¹⁰ is a substituted or unsubstituted aryl, a substituted or unsubstituted C₁-C₃ alkyl or hydrogen, in some instances hydrogen,
R¹¹ is a substituted or unsubstituted aryl, a substituted or unsubstituted C₁-C₃ alkyl, or hydrogen, in some instances a substituted or unsubstituted aryl, in some instances phenyl,
p of R¹² is 1 and R¹² is I, Br, Cl or F, in some instances Cl,
with R¹, R³, n of R²ₙ, R², R⁴, R¹⁰, R¹¹, p of R¹², R¹², R⁵ k of R⁶ₖ and R⁶ having the same meaning as defined above.

In certain embodiments, a GABA_{A} receptor modulator that can be used in the treatment of fibromyalgia and associated conditions can be a compound of general formula (2a′), (3a′), (4a′), (5a′), (5b′), (VI) or (7a′), in some instances (2a′), (3a′), (4a′), (5a′), (5b′) or (7a′), even in some instances (2a′), (3a′), (4a′), (5a′) or (5b′), or a pharmaceutically acceptable salt or solvate thereof, wherein
R¹ is
   in case of formula (2a′)
      a substituted or unsubstituted C₆ aryl, in some instances an unsubstituted phenyl,
      a substituted phenyl comprising C₁-C₄-alkyl, F, Cl, Br, I, —CN as substituents, wherein in some instances said substituted phenyl comprises at least one —F as a substituent,
   in case of formula (3a′), (5a′) or (5b′),
      a substituted or unsubstituted biphenyl, in some instances an unsubstituted biphenyl,
      a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, or
   in case of formula (4a′),
      —(C═O)—R³, with R³ being a substituted or unsubstituted C₆ heteroaryl, in some instances with R³ being pyridine,
R⁵ is
   in case of formula (7a′)
      C₂ alkinyl or I, in some instances a C₂ alkinyl, wherein R²ₙ, R¹⁰, R¹¹ and R¹², and R⁶ₖ have the same meaning as defined above.

In certain embodiments, a GABA_{A} receptor modulator that can be used in the treatment of fibromyalgia and associated conditions can be a compound of general formula (2a″), (3a″), (4a″), (5a″), (5b″), (Via″) or (7a″), in some instances (2a″), (3a″), (4a″), (5a″), (5b″) or (7a″), in some instances (2a″), (3a″), (4a″), (5a″) or (5b″), or a pharmaceutically acceptable salt or solvate thereof,

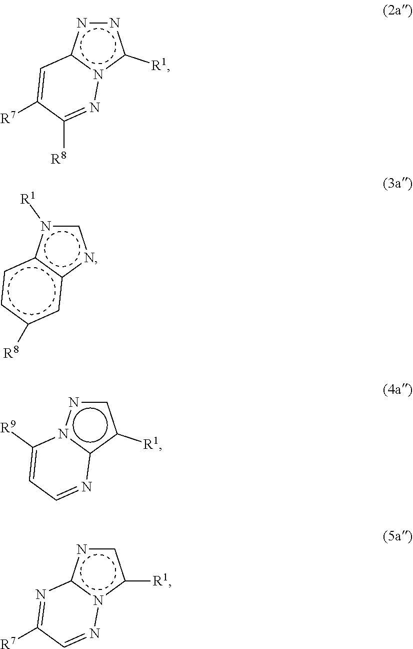

with R′ having the same meaning as defined above, and
R⁷ being
   an unsubstituted C₁-C₆ alkyl, in some instances tert-butyl,
   an unsubstituted C₃-C₈ cycloalkyl, in some instances a C₄-cycloalkyl,
   an unsubstituted C₁-C₆ alcohol, in some instances a C₃ alcohol, in some instances
      R⁷ being in case of formula (2a″)
         an unsubstituted C₁-C₆ alkyl, in some instances tert-butyl, or
         an unsubstituted C₃-C₈ cycloalkyl, in some instances a C₄-cycloalkyl, or
      R⁷ being in case of formula (5a″) or (5b″)
         an unsubstituted C₁-C₆ alcohol, in some instances a C₃ alcohol,
R⁸ being
   —O—CH₂—R⁴, with R⁴ being a substituted or unsubstituted 5-membered heteroaryl, in some instances a substituted or unsubstituted triazole, or an unsubstituted C₁-C₆ alcohol, in some instances a C₃ alcohol, in some instances
      R⁸ being in case of formula (2a″)
         —O—CH₂—R⁴, with R₄ being a substituted or unsubstituted 5-membered heteroaryl, in some instances a substituted or unsubstituted triazole, or
      R⁸ being in case of formula (3a″)
         an unsubstituted C₁-C₆ alcohol, in some instances a C₃ alcohol,
R⁹ being
   an unsubstituted C₆ heteroaryl, in some instances pyridine, or a halogen, in some instances —F,
   a halogen, in some instances —F,
      R⁹ being in case of formula (4a″)
         an unsubstituted 6-membered heteroaryl, in some instances pyridine, or
      R⁹ being in case of formula (5b″)
         a halogen, in some instances —F,
R¹⁰ being
   a C₁-C₃ alkyl or hydrogen, in some instances hydrogen
R¹¹ being a substituted or unsubstituted aryl or heteroaryl, in some instances pyridine, thiophene, a phenyl or a phenyl substituted with F or Cl,
R¹⁴ being a substituted or unsubstituted aryl or heteroaryl, in some instances a substituted or unsubstituted aryl, in some instances phenyl
R¹² being I, Cl, Br or F, in some instances Cl,
R⁵ being C₂ alkinyl or I, in some instances C₂ alkinyl.

In certain embodiments, a GABA_{A} receptor modulator that can be used in the treatment of fibromyalgia and associated conditions can be a compound of general formula (2a″), (3a″), (4a″), (5a″), (5b″), or a pharmaceutically acceptable salt or solvate thereof,

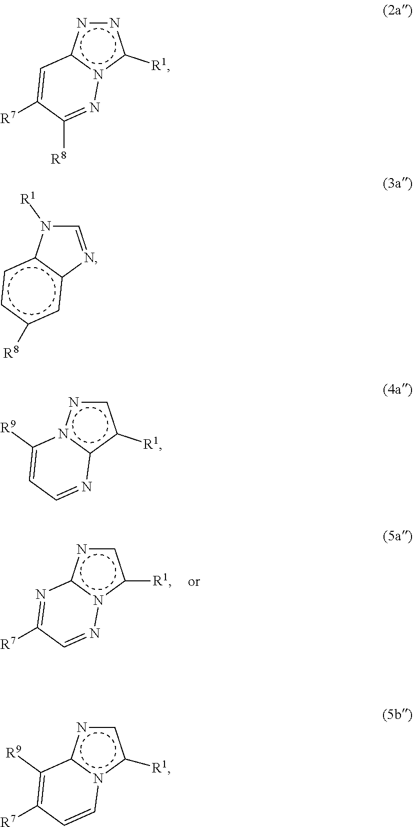

with
in case of formula (2a″)
   R¹ being
      an unsubstituted phenyl,
   a substituted phenyl comprising C₁-C₄-alkyl, F, Cl, Br, I, —CN as substituents,
   wherein in some instances said substituted phenyl comprises at least one —F as a substituent,
   R⁷ being
      an unsubstituted C₁-C₄ alkyl, in some instances tert-butyl, or
      an unsubstituted C₃-C₈ cycloalkyl, in some instances a C₄-cycloalkyl, and
   R⁸ being
      —O—CH₂—R⁴, with R⁴ being a substituted or unsubstituted 5-membered heteroaryl, in some instances a substituted or unsubstituted triazole,
in case of formula (3a″)
   R¹ being
      an unsubstituted biphenyl,
      a substituted biphenyl comprising at least one —CN as a substituent, in some instances on the phenyl moiety not connected to the parent moiety, or
      a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent,
   wherein in some instances R¹ being
      a substituted biphenyl comprising at least one —CN as a substituent, in some instances on the phenyl moiety not connected to the parent moiety,
   R⁸ being
      an unsubstituted C₁-C₆ alcohol, in some instances a C₃ alcohol, in some instances isopropanol,
in case of formula (4a″)
   R¹ being
      —(C═O)—R³, with R³ being an unsubstituted 6-membered heteroaryl, in
      particular R³ being pyridine, and
   R⁹ being
      being an unsubstituted C₆ heteroaryl, in some instances pyridine,
in case of formula (5a″)
   R¹ being
      an unsubstituted biphenyl,
      a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent,
   wherein in some instances R¹ being
      a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent,
   R⁷ being
      an unsubstituted C₁-C₆ alcohol, in some instances a C₃ alcohol, in some instances isopropanol,
in case of formula (5b″)
   R¹ being
      an unsubstituted biphenyl,
      a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent,
   wherein in some instances R¹ being
      a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent,
   R⁷ being
      an unsubstituted C₁-C₆ alcohol, in some instances a C₃ alcohol, in some instances isopropanol,
   R⁹ being
      a halogen, in some instances —F.

In certain embodiments, the compound comprises the general formula (2a″), (3a″), (5a″) or (5b″), as described above.

In certain embodiment, the compound comprises the general formula (5a″), as described above.

A GABA_{A} receptor modulator that can be used in the treatment of fibromyalgia or associated conditions can be a compound of general formula (8), or a pharmaceutically acceptable salt or solvate thereof,

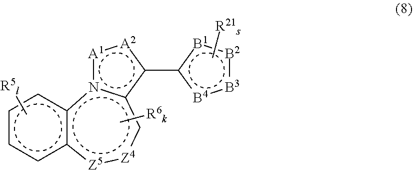

where
Z⁴ and Z⁵ are independently of each other —C, —N, —S or —O,
A¹ and A² are independently of each other —C, —N or —(C═O)—O—R⁷, with R⁷ being an alkyl,
B¹, B², B³, and B⁴ are independently of each other —C, —N, or —O,
l of R⁵ₗ is 1 or 2,
each R⁵ is independently from each other a C₁-C₄ alkinyl or a halogen, in some instances —Cl,
k of R⁶ₖ is 1, 2, 3 or 4, in some instances 1 or 2,
each R⁶ is independently from each other a substituted or unsubstituted aryl, a substituted or unsubstituted heteroaryl, a substituted or unsubstituted C₁-C₃ alkyl, oxygen or hydrogen,
s of R²¹ₛ is 1, 2, 3 or 4, or
each R²¹ is independently hydrogen or C₁-C₆ alkyl.

In some cases, a GABA_{A} receptor modulator that can be used in the treatment of pain and associated conditions can be a compound of formula (12), formula (13), formula (14), formula (15), formula (16), or formula (17), or a pharmaceutically acceptable salt or solvate thereof:

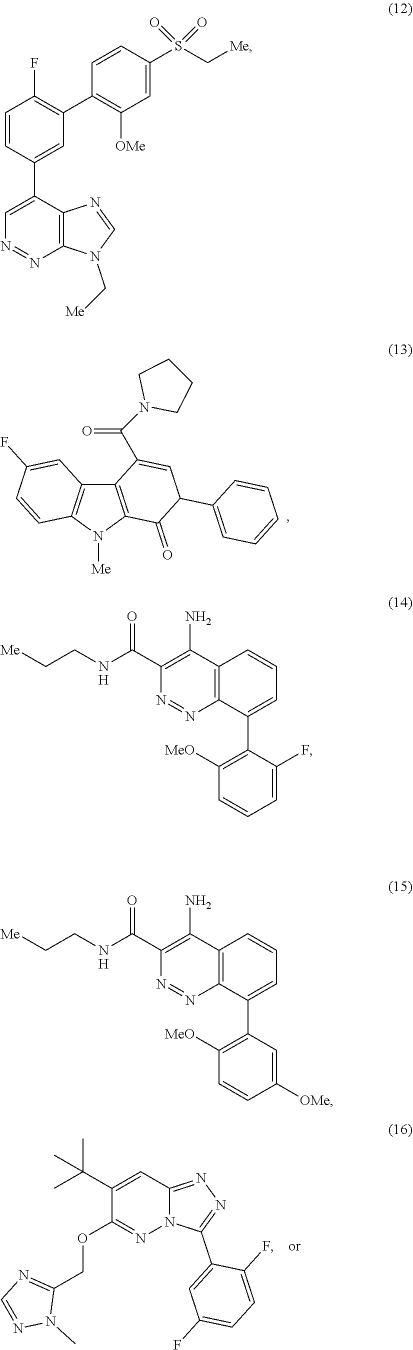

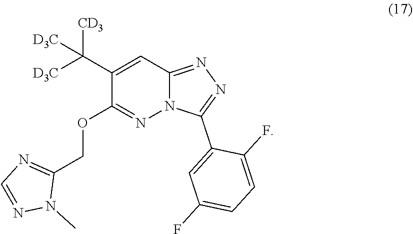 [SMILES: [2H]C([2H])([2H])C(c1cc2nnc(-c3cc(F)ccc3F)n2nc1OCc1ncnn1C)(C([2H])([2H])[2H])C([2H])([2H])[2H]]

Also contemplated herein are deuterated forms of a compound described herein. A deuterated form of a compound can include a replacement of at least one hydrogen with at least one deuterium. In some cases, a deuterated compound for use in treating pain as described herein can be a compound of formula (17), or a pharmaceutically acceptable salt or solvate thereof.

In certain embodiments, the compound is selected from the compounds depicted in FIG. 4, such as L-838417, TPA023 (MK-0777), TPA123, MRK-409(MK-0343), NS11394, Ocinaplon(DOV-273547), Compound 1, TP003, N-Desmethylclobazam 1, 2, 3, 4 and 5, Hz-166, MP-III-080, KRM-II-81, PF-06372865, SL65.1498, AZD7325, AZD6280, L-838417, CTP-354, and pharmaceutically acceptable salts or solvates thereof. In certain embodiments, the compound is Compound 1, or a pharmaceutically acceptable salt or solvate thereof,

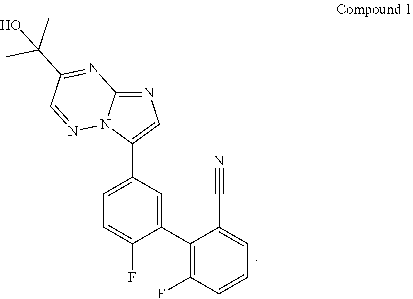 [SMILES: CC(C)(O)c1cnn2c(-c3ccc(F)c(-c4c(F)cccc4C#N)c3)cnc2n1]

In certain embodiments, a GABA_{A} receptor modulator as described herein is Compound 1.

### Pharmaceutical Compositions

Also disclosed herein are pharmaceutical compositions comprising a GABA_{A} receptor modulator as described herein. In some embodiments, a pharmaceutical composition can comprise 1, 2, 3, 4, 5, 6, 7, 8, 9, 10, 11, 12, 13, 14, 15, 16, 17, 18, 19, 20 or more GABA_{A} receptor modulators disclosed herein.

In some instances, a pharmaceutical composition can comprise a GABA_{A} receptor modulator described herein and at least one of: an excipient, a diluent, or a carrier. In some cases, a GABA_{A} receptor modulator described herein can be dissolved or suspended in a diluent or carrier.

In some embodiments, a pharmaceutical composition can comprise an excipient. An excipient can be an excipient described in the Handbook of Pharmaceutical Excipients, American Pharmaceutical Association (1986).

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In some embodiments an excipient can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical composition.

In some embodiments an excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments a pharmaceutical composition can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

The binders that can be used in a pharmaceutical composition can be selected from starches such as potato starch, corn starch, wheat starch; sugars such as sucrose, glucose, dextrose, lactose, maltodextrin; natural and synthetic gums; gelatin; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments a pharmaceutical composition can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical composition can be selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulfates (such as sodium lauryl sulfate, magnesium lauryl sulfate), sodium chloride, sodium benzoate, sodium acetate and talc or a combination thereof.

In some embodiments a pharmaceutical composition can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments a pharmaceutical composition can comprise a disintegrant as an excipient. In some embodiments a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments a disintegrant can be an effervescent disintegrant. Non-limiting examples of suitable effervescent disintegrants can include sodium bicarbonate in combination with citric acid, and sodium bicarbonate in combination with tartaric acid.

In some embodiments an excipient can comprise a flavoring agent. Flavoring agents incorporated into an outer layer can be chosen from synthetic flavor oils and flavoring aromatics; natural oils; extracts from plants, leaves, flowers, and fruits; and combinations thereof. In some embodiments a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments an excipient can comprise a sweetener. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, sylitol, and the like.

In some instances, a pharmaceutical composition can comprise a coloring agent. Non-limiting examples of suitable color agents can include food, drug and cosmetic colors (FD&C), drug and cosmetic colors (D&C), and external drug and cosmetic colors (Ext. D&C). A coloring agent can be used as dyes or their corresponding lakes.

In some instances, a pharmaceutical composition can comprise a diluent. Non-limiting examples of diluents can include water, glycerol, methanol, ethanol, and other similar biocompatible diluents. In some cases, a diluent can be an aqueous acid such as acetic acid, citric acid, maleic acid, hydrochloric acid, phosphoric acid, nitric acid, sulfuric acid, or similar. In some instances, a diluent can be used to titrate a pH of a compound to a pH such as physiological pH to produce a salt as described above. In other cases, a diluent can be selected from a group comprising alkaline metal carbonates such as calcium carbonate; alkaline metal phosphates such as calcium phosphate; alkaline metal sulfates such as calcium sulfate; cellulose derivatives such as cellulose, microcrystalline cellulose, cellulose acetate; magnesium oxide, dextrin, fructose, dextrose, glyceryl palmitostearate, lactitol, caoline, lactose, maltose, mannitol, simethicone, sorbitol, starch, pregelatinized starch, talc, xylitol and/or anhydrates, hydrates and/or pharmaceutically acceptable derivatives thereof or combinations thereof.

In other embodiments, a pharmaceutical composition can comprise a surfactant. Surfactants can be selected from, but not limited to, polyoxyethylene sorbitan fatty acid esters (polysorbates), sodium lauryl sulfate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids or a combination thereof.

A pharmaceutical composition disclosed herein can be formulated into a variety of forms and administered by a number of different means. A pharmaceutical composition can be administered orally, rectally, or parenterally, in formulations containing conventionally acceptable carriers, adjuvants, and vehicles as desired. The term “parenteral” as used herein can include subcutaneous, intravenous, intramuscular, or intrasternal injection and infusion techniques. Administration can include injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration. In some exemplary embodiments, a route of administration can be via an injection such as an intramuscular, intravenous, subcutaneous, or intraperitoneal injection.

Solid dosage forms for oral administration can include capsules, tablets, caplets, pills, troches, lozenges, powders, and granules. A capsule can comprise a core material comprising a nutritive protein or composition and a shell wall that encapsulates a core material. In some embodiments a core material can comprise at least one of a solid, a liquid, and an emulsion. In some embodiments a shell wall material can comprise at least one of a soft gelatin, a hard gelatin, and a polymer. Suitable polymers can include but not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer can function as taste-masking agents.

Tablets, pills, and the like can be compressed, multiply compressed, multiply layered, and/or coated. A coating can be single or multiple. In some embodiments, a coating material can comprise at least one of a saccharide, a polysaccharide, and glycoproteins extracted from at least one of a plant, a fungus, and a microbe. Non-limiting examples can include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments a coating material can comprise a protein. In some embodiments, a coating material can comprise at least one of a fat and/or an oil. In some embodiments the at least one of a fat and/or an oil can be high temperature melting. In some embodiments the at least one of a fat and/or an oil can be hydrogenated or partially hydrogenated. In some embodiments the at least one of a fat and/or an oil can be derived from a plant. In some embodiments the at least one of a fat and/or an oil can comprise at least one of glycerides, free fatty acids, and fatty acid esters. In some embodiments a coating material can comprise at least one edible wax. An edible wax can be derived from animals, insects, or plants. Non-limiting examples can include beeswax, lanolin, bayberry wax, carnauba wax, and rice bran wax. Tablets and pills can additionally be prepared with enteric coatings.

Liquid formulations can include a syrup (for example, an oral formulation), an intravenous formulation, an intranasal formulation, an ocular formulation (e.g. for treating an eye infection), an otic formulation (e.g. for treating an ear infection), an ointment, a cream, an aerosol, and the like. In some instances, a combination of various formulations can be administered. In some embodiments, a tablet, pill, and the like can be formulated for an extended release profile.

Dosing and Administration

In some cases, a GABA_{A} receptor modulator, salt thereof, or pharmaceutical composition comprising a GABA_{A} receptor modulator or salt thereof described herein can be administered at a dose of from about 1 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 35 mg to about 1000 mg, from about 40 mg to about 1000 mg, from about 45 mg to about 1000 mg, from about 50 mg to about 1000 mg, from about 55 mg to about 1000 mg, from about 60 mg to about 1000 mg, from about 65 mg to about 1000 mg, from about 70 mg to about 1000 mg, from about 75 mg to about 1000 mg, from about 80 mg to about 1000 mg, from about 85 mg to about 1000 mg, from about 90 mg to about 1000 mg, from about 95 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg.

In some cases, a GABA_{A} receptor modulator, salt or solvate thereof, or pharmaceutical composition comprising a GABA_{A} receptor modulator or salt thereof described herein can be administered at a dose of about 0.01, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 mg.

In some cases, a GABA_{A} receptor modulator, salt or solvate thereof, or pharmaceutical composition comprising a GABA_{A} receptor modulator or salt thereof described herein can be administered at a dose with respect to a subject body weight. In some cases, a GABA_{A} receptor modulator, salt or solvate thereof, or pharmaceutical composition comprising a GABA_{A} receptor modulator or salt or solvate thereof described herein can be administered at a dose of from about 0.05 mg/kg to about 100 mg/kg, from about 0.005 mg/kg to about 95 mg/kg, from about 0.005 mg/kg to about 90 mg/kg, from about 0.005 mg/kg to about 85 mg/kg, from about 0.005 mg/kg to about 80 mg/kg, from about 0.005 mg/kg to about 75 mg/kg, from about 0.005 mg/kg to about 70 mg/kg, from about 0.005 mg/kg to about 65 mg/kg, from about 0.005 mg/kg to about 60 mg/kg, from about 0.005 mg/kg to about 55 mg/kg, from about 0.005 mg/kg to about 50 mg/kg, from about 0.005 mg/kg to about 45 mg/kg, from about 0.005 mg/kg to about 40 mg/kg, from about 0.005 mg/kg to about 35 mg/kg, from about 0.005 mg/kg to about 30 mg/kg, from about 0.005 mg/kg to about 25 mg/kg, from about 0.005 mg/kg to about 20 mg/kg, from about 0.005 mg/kg to about 15 mg/kg, or from about 0.005 mg/kg to about 10 mg/kg, with respect to a body weight of a subject. In some cases, a GABA_{A} receptor modulator, salt or solvate thereof, or pharmaceutical composition comprising a GABA_{A} receptor modulator or salt or solvate thereof described herein can be administered at a dose of from about 0.2 mg/kg to about 10 mg/kg, from about 0.2 mg/kg to about 9 mg/kg, from about 0.2 mg/kg to about 8 mg/kg, from about 0.2 mg/kg to about 7 mg/kg, from about 0.2 mg/kg to about 6 mg/kg, from about 0.2 mg/kg to about 5 mg/kg, from about 0.2 mg/kg to about 4 mg/kg, from about 0.2 mg/kg to about 3 mg/kg, from about 0.2 mg/kg to about 2 mg/kg, or from about 0.2 mg/kg to about 1 mg/kg, with respect to a body weight of a subject. In some cases, a GABA_{A} receptor modulator, salt or solvate thereof, or pharmaceutical composition comprising a GABA_{A} receptor modulator or salt or solvate thereof described herein can be administered at a dose of from about 0.001 mg/kg, about 0.002 mg/kg, about 0.003 mg/kg, about 0.004 mg/kg, or about 0.005 mg/kg to about 0.01 mg/kg, about 0.02 mg/kg, about 0.03 mg/kg, about 0.04 mg/kg, about 0.05 mg/kg, about 0.1 mg/kg, or about 1 mg/kg, with respect to a body weight of a subject.

In some cases, a GABA_{A} receptor modulator, salt or solvate thereof, or pharmaceutical composition comprising a GABA_{A} receptor modulator or salt thereof described herein can be administered at a dose of less than about 0.001 mg/kg, less than 0.002 mg/kg, less than 0.003 mg/kg, less than 0.004 mg/kg, less than 0.005 mg/kg, less than 0.006 mg/kg, less than 0.007 mg/kg, less than 0.008 mg/kg, less than 0.009 mg/kg, less than 0.01 mg/kg, less than 0.02 mg/kg, less than 0.03 mg/kg, less than 0.04 mg/kg, less than 0.05 mg/kg, less than 0.06 mg/kg, less than 0.07 mg/kg, less than 0.08 mg/kg, less than 0.09 mg/kg, less than 0.1 mg/kg, less than 0.2 mg/kg, less than 0.3 mg/kg, less than 0.4 mg/kg, less than 0.5 mg/kg, less than 0.6 mg/kg, less than 0.7 mg/kg, less than 0.8 mg/kg, less than 0.9 mg/kg, less than 1 mg/kg, less than 1.1 mg/kg, less than 1.2 mg/kg, less than 1.3 mg/kg, less than 1.4 mg/kg, less than 1.5 mg/kg, less than 1.6 mg/kg, less than 1.7 mg/kg, less than 1.8 mg/kg, less than 1.9 mg/kg, less than 2 mg/kg, less than 2.1 mg/kg, less than 2.2 mg/kg, less than 2.3 mg/kg, less than 2.4 mg/kg, less than 2.5 mg/kg, less than 2.6 mg/kg, less than 2.7 mg/kg, less than 2.8 mg/kg, less than 2.9 mg/kg, less than 3 mg/kg, less than 3.1 mg/kg, less than 3.2 mg/kg, less than 3.3 mg/kg, less than 3.4 mg/kg, less than 3.5 mg/kg, less than 3.6 mg/kg, less than 3.7 mg/kg, less than 3.8 mg/kg, less than 3.9 mg/kg, less than 4 mg/kg, less than 4.1 mg/kg, less than 4.2 mg/kg, less than 4.3 mg/kg, less than 4.4 mg/kg, less than 4.5 mg/kg, less than 4.6 mg/kg, less than 4.7 mg/kg, less than 4.8 mg/kg, less than 4.9 mg/kg, less than 5 mg/kg, less than 5.1 mg/kg, less than 5.2 mg/kg, less than 5.3 mg/kg, less than 5.4 mg/kg, less than 5.5 mg/kg, less than 5.6 mg/kg, less than 5.7 mg/kg, less than 5.8 mg/kg, less than 5.9 mg/kg, less than 6 mg/kg, less than 6.1 mg/kg, less than 6.2 mg/kg, less than 6.3 mg/kg, less than 6.4 mg/kg, less than 6.5 mg/kg, less than 6.6 mg/kg, less than 6.7 mg/kg, less than 6.8 mg/kg, less than 6.9 mg/kg, less than 7 mg/kg, less than 7.1 mg/kg, less than 7.2 mg/kg, less than 7.3 mg/kg, less than 7.4 mg/kg, less than 7.5 mg/kg, less than 7.6 mg/kg, less than 7.7 mg/kg, less than 7.8 mg/kg, less than 7.9 mg/kg, less than 8 mg/kg, less than 8.1 mg/kg, less than 8.2 mg/kg, less than 8.3 mg/kg, less than 8.4 mg/kg, less than 8.5 mg/kg, less than 8.6 mg/kg, less than 8.7 mg/kg, less than 8.8 mg/kg, less than 8.9 mg/kg, less than 9 mg/kg, less than 9.1 mg/kg, less than 9.2 mg/kg, less than 9.3 mg/kg, less than 9.4 mg/kg, less than 9.5 mg/kg, less than 9.6 mg/kg, less than 9.7 mg/kg, less than 9.8 mg/kg, less than 9.9 mg/kg, less than 10 mg/kg, less than 11 mg/kg, less than 12 mg/kg, less than 13 mg/kg, less than 14 mg/kg, less than 15 mg/kg, less than 16 mg/kg, less than 17 mg/kg, less than 18 mg/kg, less than 19 mg/kg, less than 20 mg/kg, less than 21 mg/kg, less than 22 mg/kg, less than 23 mg/kg, less than 24 mg/kg, less than 25 mg/kg, less than 26 mg/kg, less than 27 mg/kg, less than 28 mg/kg, less than 29 mg/kg, less than 30 mg/kg, less than 31 mg/kg, less than 32 mg/kg, less than 33 mg/kg, less than 34 mg/kg, less than 35 mg/kg, less than 36 mg/kg, less than 37 mg/kg, less than 38 mg/kg, less than 39 mg/kg, less than 40 mg/kg, less than 41 mg/kg, less than 42 mg/kg, less than 43 mg/kg, less than 44 mg/kg, less than 45 mg/kg, less than 46 mg/kg, less than 47 mg/kg, less than 48 mg/kg, less than 49 mg/kg, or less than 50 mg/kg. In some cases, a GABA_{A} receptor modulator, salt or solvate thereof, or pharmaceutical composition comprising a GABA_{A} receptor modulator or salt thereof described herein can be administered at a dose of at most about 0.001 mg/kg, at most about 0.002 mg/kg, at most about 0.003 mg/kg, at most about 0.004 mg/kg, at most about 0.005 mg/kg, at most about 0.006 mg/kg, at most about 0.007 mg/kg, at most about 0.008 mg/kg, at most about 0.009 mg/kg, at most about 0.01 mg/kg, at most about 0.02 mg/kg, at most about 0.03 mg/kg, at most about 0.04 mg/kg, at most about 0.05 mg/kg, at most about 0.06 mg/kg, at most about 0.07 mg/kg, at most about 0.08 mg/kg, at most about 0.09 mg/kg, at most about 0.1 mg/kg, at most about 0.2 mg/kg, at most about 0.3 mg/kg, at most about 0.4 mg/kg, at most about 0.5 mg/kg, at most about 0.6 mg/kg, at most about 0.7 mg/kg, at most about 0.8 mg/kg, at most about 0.9 mg/kg, at most about 1 mg/kg, at most about 1.5 mg/kg, at most about 2.0 mg/kg or at most about 3.0 mg/kg.

Administration of a GABA_{A} receptor modulator, salt or solvate thereof, or composition can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration of a GABA_{A} receptor modulator, salt or solvate thereof, or composition can be performed at least 1, 2, 3, 4, 5, 6, 7, 8, 9, 10, 11, 12, 13, 14, 15, 16, 17, 18, 19, 20, or 21 times a week. In some cases, administration of a GABA_{A} receptor modulator, salt or solvate thereof, or composition can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

The invention is further illustrated by the following examples, from which further embodiments and advantages can be drawn. These examples are meant to illustrate the invention but not to limit its scope.

### Example 1—Formulation

An exemplary GABA_{A} receptor modulator was formulated prior to administration. The exemplary GABA_{A} receptor modulator was provided as a yellow powder at a purity of 98.87%. The dry solid was dissolved in 0.5% methyl cellulose in water. Various concentrations of the exemplary GABA_{A} receptor modulator were prepared. For example, a 0.6 mg/mL test article was prepared by dissolving 10.9 mg of the phosphate salt of the exemplary GABA_{A} receptor modulator in 14.5 mL of the 0.5% methyl cellulose.

Control solutions of 0.5% methyl cellulose and pregabalin in phosphate buffered saline (PBS) were prepared and set aside for comparison.

### Example 2—Study Design

The purpose of the study is to assess the efficacy of the described compounds in the rat model of fibromyalgia pain.

Inclusion Criteria:

After the pre-treatment baseline assessment on Day 0, only animals that exhibit at least a 20% decrease in thresholds from pre-injury baseline to pre-dosing baseline were included in the study.

Blinding:

All testing was performed in a blinded manner, with all experimenters involved in the study being unaware of the group assignment of any animal they were testing. One staff member prepared the dose formulations, coded the syringes of solutions (e.g. 1-5), and created the blind (treatment key).

Group Assignment:

Animals were assigned to treatment groups based on Day 0 pre-dose von Frey 50% response thresholds so that group means of the mean paw withdrawal latencies were approximately equal. Animals were ranked by pre- and post-reserpine baseline change from lowest to highest, and treatments were assigned randomly within stratified sub-groups according to the total number of treatment groups in the study.

Dosing:

The volume of compound 1 or control article injected was 5 mL/kg. The animals were dosed in sequence based on animal number, so that the distribution of treatment across a given set of animals was not predictable.

Animal Model

Rats were the model of choice for the study of pain due to many similarities of the peripheral and central nervous systems of rats and humans. These similarities are evident both in terms of behavioral responses to painful conditions and in terms of pain relieving effects of various therapeutic agents (i.e. opioids, nonsteroidal anti-inflammatory drugs, anticonvulsants and antidepressants) in both species. Rats are among the best species for determining the predictability of efficacy of therapeutic agents in humans. Further, given the primary goal of these studies is to investigate the pain causing effects of compounds, it is necessary to use vertebrate animals when using whole animal models.

A total of 54 rats were requested for this study to ensure that enough animals were prepared that meet study criteria (n=10 per group, 4 groups). 14 more animals were requested for reserpine treatment as a success rate of ˜70% was expected, i.e., ˜30% of the rats treated with reserpine either are euthanized early for weight loss or do not meet the study inclusion criteria for mechanical sensitivity. Therefore, to ensure 10 rats per study group (n=40), 54 animals were treated with reserpine.

The appropriate group size (n=10) was selected to detect a response difference of 2.5 units with a power of 80% (standard deviation=3). The number of animals requested was consistent with the sample group sizes reported in peer reviewed research articles and in previous studies. Previous experience with the model indicates that a group size of at least 10 animals per group was required to generate interpretable and reproducible results.

50% Response Threshold (g)

Mechanical fibromyalgia-associated allodynia is measured using 8 Semmes-Weinstein filaments (Stoelting©; Wood Dale, Ill., USA) with varying stiffness (0.04, 0.07, 0.16, 0.4, 1.0, 4.0, and 8.0 g) according to the up-down method 1. Animals were placed in individual acrylic chambers on a metal mesh surface and allowed to acclimate to their surroundings for a minimum of 15 minutes before testing. Each filament was presented perpendicular to the plantar surface with sufficient force to cause slight buckling against the paw and held for approximately 4 seconds or until a positive response is noted (paw sharply withdrawn). Testing was initiated with the 0.4 g filament. In the absence of a paw withdrawal response, the next stronger stimulus was presented. In the event of paw withdrawal, the next weaker stimulus was used. This process was repeated until 4 responses after the initial change in response (no response to positive response or positive response to no response) were obtained. If the animal does not respond after reaching the strongest filament or if the animal responded after reaching the weakest filament, the testing is stopped for that time point. The 50% response threshold is calculated using the formula:

50% response threshold (g)=(10(Xf+kδ))/10,000

Xf=value (in log units) of the final von Frey filament used

k=tabular value for the pattern of positive/negative responses (Chaplan et al. 1994, appendix 1, page 62)

δ=mean difference (in log units) between stimuli

The mean and standard error of the mean (SEM) is determined for each paw for each treatment group at each time point.

Experimental Timeline

### Day −7:

Pre-Injury mechanical fibromyalgia-associated allodynia testing (vF)
Reserpine injection

### Day −6:

Reserpine injection

### Day −5:

Reserpine injection

### Day 0:

Mechanical fibromyalgia-associated allodynia testing (vF; time baseline)
Dosing (time 0 min)
Mechanical fibromyalgia-associated allodynia testing (vF; time ˜1 hour)
Mechanical fibromyalgia-associated allodynia testing (vF; time ˜2 hour)
Mechanical fibromyalgia-associated allodynia testing (vF; time ˜4 hour)
Euthanasia

Dosing Regimen

| Group | | | Dose | Dose Vol. | | Day of Admin./ |
|---|---|---|---|---|---|---|
| # | Treatment | N | (mg/kg) | (ml/kg) | Route | Frequency |
| 1 | Vehicle | 10 | 0 | 5 | PO | Day 0/SID |
| 2 | Compound 1 | 10 | 0.3 | 5 | PO | Day 0/SID |
| 3 | Compound 1 | 10 | 3 | 5 | PO | Day 0/SID |
| 4 | Pregabalin | 10 | 30 | 5 | PO | Day 0/SID |

Statistical Analysis

TABLE 1
Development of Persistent Pain - Statistical Table

| Subject | | Parametric Statistical Analysis Methods for |
|---|---|---|
| name(s) | Group(s) | Hg Testing |
| Assess Pain | 1 | Analysis: Wilcoxon matched-pairs signed-rank |
| State | | test |
| | | Post-hoc test: NA |
| | | Comparison: NA |
| | | Time Point(s): Pre-injury vs. Pre-dose |
| Assess | 1, 2, 3 | Analysis: Kruskal-Wallis one-way ANOVA |
| Effect of | | Post-hoc test: Dunn's |
| Test Articles | | Comparison: Ipsilateral Group 2, 3 vs. vehicle |
| | | Group 1 |
| | | Time Point(s): All |
| Assess | 1, 4 | Analysis: Mann-Whitney test |
| Effect of | | Post-hoc test: NA |
| Positive | | Comparison: Ipsilateral Group 4 vs. vehicle |
| Control | | Group 1 |
| Article | | Time Point(s): All |

The level of significance for all analyses was set at p = 0.05.

### Example 3—Treatment of Fibromyalgia-Associated Allodynia

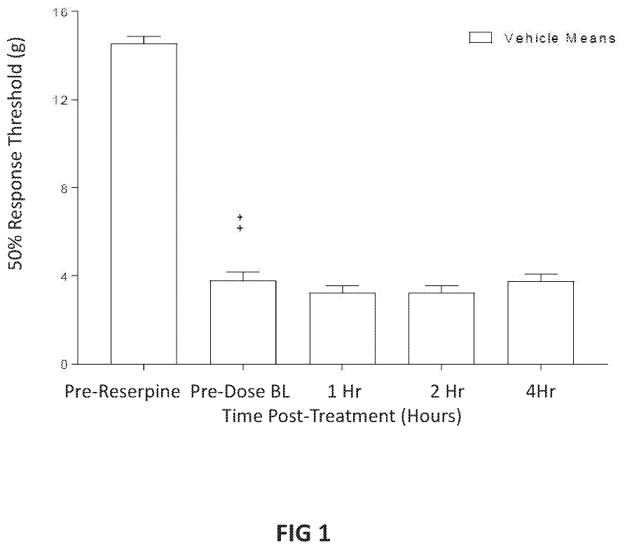

To verify the development of mechanical fibromyalgia-associated allodynia due to reserpine treatment, median ipsilateral and contralateral paw response thresholds from the vehicle group were assessed prior to dosing and at post-dosing baseline. Mean response thresholds were compared using a Wilcoxon matched-pairs signed-rank test. Pre-dose baseline tests were significantly lower (FIG. 1), indicating persistent pain due to reserpine injections. The pre-reserpine baseline versus day 0 baseline post-reserpine had a P value of 0.0020.

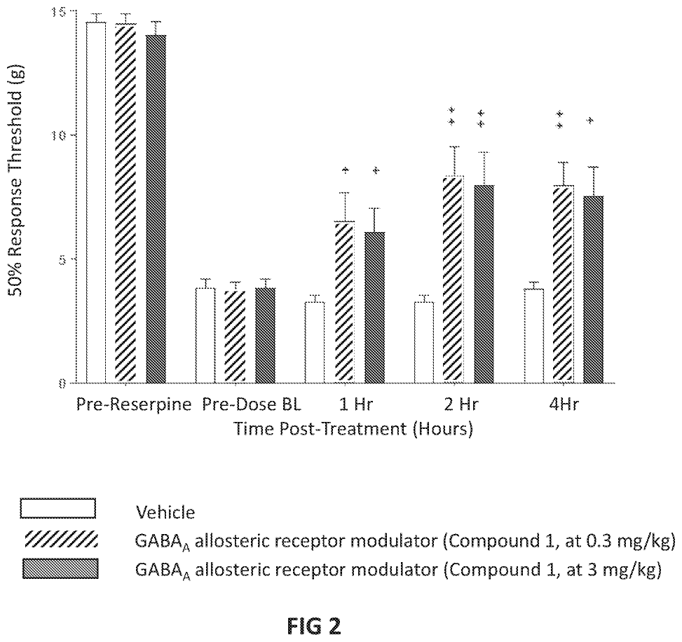

Four days after reserpine injections (study day 0), mechanical fibromyalgia-associated allodynia was assessed at baseline (prior to dosing) and 1, 2, and 4 hours post-dosing with either vehicle or the test article, an exemplary GABA_{A} receptor modulator. FIG. 2 illustrates that oral administration of an exemplary GABA_{A} receptor modulators (i.e., Compound 1) at 0.3 mg/kg and 3 mg/kg each produced a significant reversal of mechanical fibromyalgia-associated allodynia induced by reserpine treatment across the tested time points. Results for the exemplary GABA_{A} receptor modulator of the present disclosure, i.e., Compound 1, are illustrated in Table 2 below.

TABLE 2
Assessment of Test Article Administration,
Pharmacological Assessment Period - Statistical Table
Kruskal-Wallis, two-tailed, Vehicle,
Compound 1 (0.3 mg/kg), Compound 1 (3 mg/kg)

| | | | Dunn's Multiple Comparison | |
|---|---|---|---|---|
| | | | vs Vehicle (Adjusted P Value) | |
| Kruskal-Wallis statistic | | | Compound 1 | Compound 1 |
| Time Point | K | P Value | 0.3 mg/kg | 3 mg/kg |
| Day −7, Baseline | 0.5833 | 0.9002 | >0.9999 | >0.9999 |
| Day 0, Baseline | 0.0400 | 0.9979 | >0.9999 | >0.9999 |
| Day 0, 1-hour | 7.727 | 0.0210 | 0.0769 | 0.0713 |
| Day 0, 2-hour | 12.44 | 0.0020 | 0.0033 | 0.0064 |
| Day 0, 4-hour | 10.31 | 0.0058 | 0.0042 | 0.0388 |

### Example 4—Positive Control

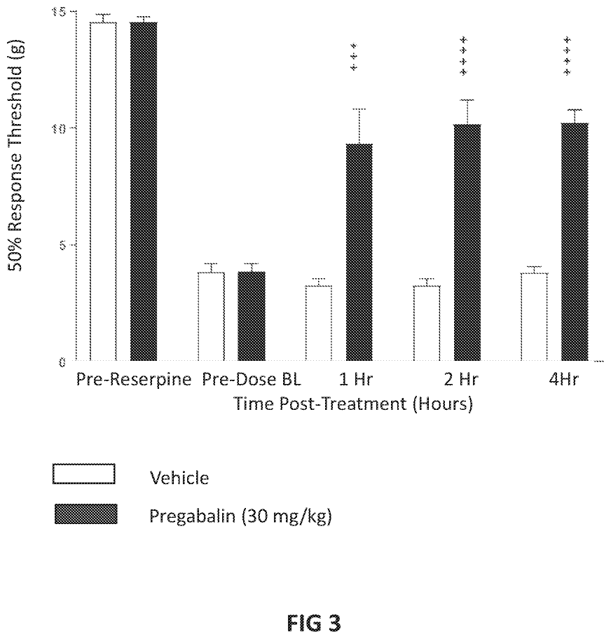

Four days after reserpine injections (study day 0), mechanical fibromyalgia-associated allodynia was assessed at baseline (prior to dosing) and approximately 1, 2, and 4 hours after administration of vehicle, compound 1, or pregabalin. FIG. 3 illustrates that oral Administration of pregabalin (30 mg/kg) significantly increased median response thresholds at all of the time points following dosing. The results are tabulated in Table 3 below.

TABLE 3
Development of Persistent Pain - Statistical Table
Unpaired t-test, two-tailed, Ipsilateral vs. Contralateral

| Time Point | Mann-Whitney U | P Value |
|---|---|---|
| Pre-reserpine BL | 46.5 | >0.9999 |
| Day 0 BL | 49.5 | 0.9871 |
| Day 0, 1-hr | 8 | 0.0007 |
| Day 0, 2-hr | 0 | <0.0001 |
| Day 0, 4 hr | 0 | <0.0001 |

### Example 5—Comparative Analysis

Methods were carried out as described as in Examples 2-4. The mean paw response thresholds for each treatment regimen are depicted in Table 4 below:

TABLE 4
Paw Response Threshold - Means and SEM
Mean Paw Response Thresholds (g) - Pharmacological Assessment

| Time Point | Treatment | Mean | SEM |
|---|---|---|---|
| Pre-reserpine | Vehicle | 14.55 | 0.3202 |
| | 0.3 mg/kg Compound 1 | 14.50 | 0.3651 |
| | 3 mg/kg Compound 1 | 14.01 | 0.5664 |
| | 30 mg/kg pregabalin | 14.55 | 0.2991 |
| Day 0, Baseline | Vehicle | 3.82 | 0.3756 |
| | 0.3 mg/kg Compound 1 | 3.68 | 0.3969 |
| | 3 mg/kg Compound 1 | 3.82 | 0.3820 |
| | 30 mg/kg pregabalin | 3.84 | 0.3625 |
| Day 0, 1 hr | Vehicle | 3.56 | 0.3106 |
| | 0.3 mg/kg Compound 1 | 6.52 | 1.144 |
| | 3 mg/kg Compound 1 | 6.08 | 0.9543 |
| | 30 mg/kg pregabalin | 9.34 | 1.485 |
| Day 0, 2 hr | Vehicle | 3.26 | 0.2813 |
| | 0.3 mg/kg Compound 1 | 8.36 | 1.147 |
| | 3 mg/kg Compound 1 | 7.98 | 1.331 |
| | 30 mg/kg pregabalin | 10.17 | 1.021 |
| Day 0, 4 hr | Vehicle | 3.79 | 0.2763 |
| | 0.3 mg/kg Compound 1 | 7.96 | 0.9253 |
| | 3 mg/kg Compound 1 | 7.54 | 1.157 |
| | 30 mg/kg pregabalin | 10.23 | 0.5544 |

Discussion

Pain was induced by three daily doses of 1 mg/kg reserpine, and mechanical fibromyalgia-associated allodynia was assessed using von Frey filaments. Mechanical fibromyalgia-associated allodynia was measured prior to and the first reserpine injection and 4 days following the third injection. GABA_{A} receptor modulators (0.3 or 3 mg/kg), pregabalin (30 mg/kg) or vehicle (0.5% methylcellulose) were administered 4 days after the last reserpine injection, with mechanical fibromyalgia-associated allodynia being assessed prior to, and at 1, 2, and 4 hours post-administration to determine the efficacy of GABA_{A} receptor modulators in this model.

The objective of this study was to assess the efficacy of GABA_{A} positive allosteric receptor modulators at two doses against fibromyalgia pain in a rat model. 40 animals in this study met the inclusion criteria after pre-dose baseline testing, operationally defined as at least a 20% decrease in thresholds from pre-injury baseline to pre-dosing baseline. Mechanical fibromyalgia-associated allodynia persisted in vehicle-treated animals throughout the pharmacological assessment period (1, 2, and 4 hours post-dosing).

Administration of the positive control, pregabalin (30 mg/kg, PO), significantly reversed mechanical fibromyalgia-associated allodynia at the 1, 2, and 4 hour time points.

There was significant reduction in mechanical fibromyalgia-associated allodynia observed at 1, 2, and 4 hours post-dosing orally for both GABA_{A} receptor modulator concentrations (0.3 or 3 mg/kg). The efficacy of the two doses were generally similar at all time points, and did not appear to be waning at either dose level at the final (4 hour) time point. Accordingly, it is understandable that multiple dosages are tolerated and effective across a wide range. As such, additional dosages are described above in the section titled Dosing and Administration.

In summary, this study is valid with significant fibromyalgia-associated allodynia induced during the model creation process that was reversible by the administration of the positive control, pregabalin (30 mg/kg. P0). Administration of GABA_{A} receptor modulators at both 0.3 mg/kg and 3 mg/kg significantly reversed the fibromyalgia-associated allodynia seen in this model using the dosing regimen and time points tested in this study.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating fibromyalgia comprising administering to a subject in need thereof a therapeutically effective amount of a compound that is a GABA_{A} receptor modulator.

2. The method of claim 1, wherein the compound is a positive GABA_{A} receptor modulator.

3. The method of claim 1, wherein the compound is a positive allosteric GABA_{A} receptor modulator.

4. The method of claim 1, wherein the compound is an α1, α2, α3, or α5 GABA_{A} receptor modulator.

5. The method of claim 4, wherein the compound is a positive allosteric α2 or α3 GABA_{A} receptor modulator.

6. The method of claim 1, wherein the compound is of the general formula (1a), general formula (1b), general formula (1c), or a pharmaceutically acceptable salt or solvate thereof, 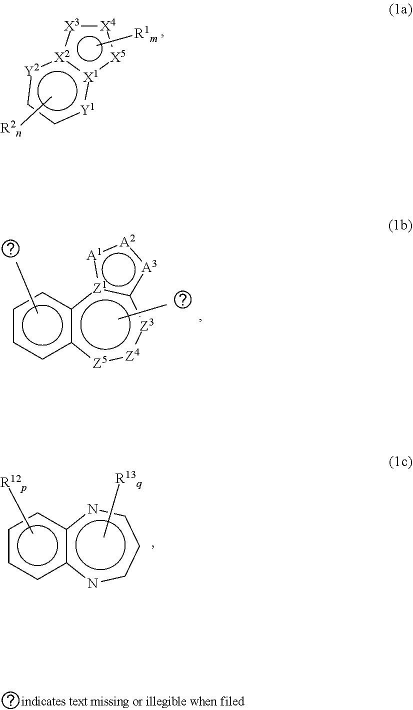
wherein
X¹, X², X³, X⁴ and X⁵ are each independently —C, —N, —S or —O, wherein at least two of X¹, X², X³, X⁴ and X⁵ are —N,
Y¹ and Y² are each independently —C or —N,
m of R¹ₘ is 1, wherein R¹ is an unsubstituted phenyl, a phenyl substituted with C₁-C₄-alkyl, F, Cl, Br, I, —CN, a substituted or unsubstituted biphenyl or —(C═O)—R³, wherein R³ is a substituted or unsubstituted aryl or 5- to 6-membered heteroaryl,
n of R²ₙ is 1 or 2, wherein each R² is independently a substituted or unsubstituted C₃-C₈ cycloalkyl, a substituted or unsubstituted C₁-C₆ alkyl, a substituted or unsubstituted C₁-C₆ alcohol, a substituted or unsubstituted 6-membered heteroaryl, a halogen, or —O—CH₂—R⁴, wherein R⁴ is a substituted or unsubstituted 5- or 6-membered heteroaryl,
Z¹, Z³, Z⁴, and Z⁵ are each independently —C, —N, —S or —O,
A¹, A² and A³, are each independently —C, —N, —C(C═O)—O—R⁷, or 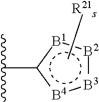
wherein
R⁷ is alkyl,
B¹, B², B³, and B⁴ are each independently —C, —N, or —O,
s of R²¹ₛ is 1, 2, 3 or 4, and
R²¹ is hydrogen or C₁-C₆ alkyl,
l of R⁵ₗ is 1 or 2, wherein each R⁵ is independently a C₁-C₄ alkinyl or a halogen,
k of R⁶ₖ is 1, 2, 3 or 4, wherein each R⁶ is independently a substituted or unsubstituted aryl, a substituted or unsubstituted heteroaryl, a substituted or unsubstituted C₁-C₃ alkyl, or hydrogen,
p of R¹²ₚ is 1 or 2, wherein each R¹² is independently a substituted or unsubstituted C₁-C₄-alkyl, I, Br, Cl or F, and
q of R¹³_{q} is 1, 2, 3 or 4, wherein each R¹³ is independently a substituted or unsubstituted aryl, a substituted or unsubstituted heteroaryl, a substituted or unsubstituted C₁-C₃ alkyl, oxygen or hydrogen.

7. The method of claim 6, wherein the compound is of the general formula (2), (3), (4), (5), (1c), (7), or a pharmaceutically acceptable salt or solvate thereof,

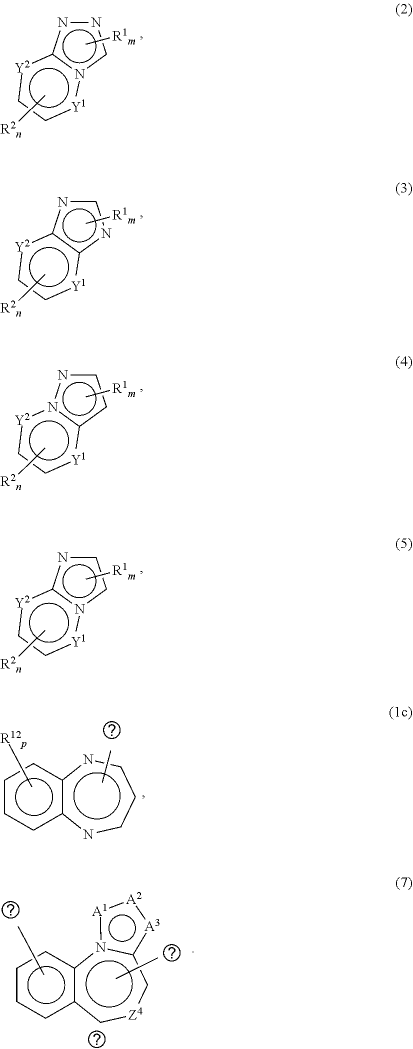

8. The method of claim 6, wherein the compound is of the general formula (2a), (3a), (4a), (5a), (5b), (1c), (7a), or a pharmaceutically acceptable salt or solvate thereof,

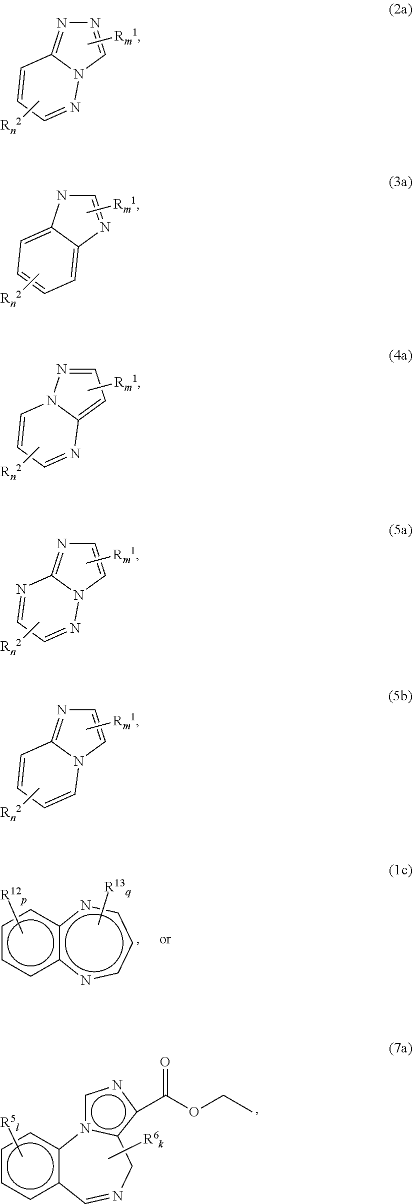

9. The method of claim 6, wherein
m of R¹ₘ is 1, and wherein R¹ is:
an unsubstituted phenyl,
a substituted phenyl comprising C₁-C₄-alkyl, F, Cl, Br, I, —CN as substituents,
an unsubstituted biphenyl,
a substituted biphenyl comprising at least one —CN as a substituent,
a substituted biphenyl comprising at least one —CN as a substituent, or —(C═O)—R³, wherein R³ is pyridine.

10. The method of claim 6, wherein the compound is of the general formula (2a′), (3a′), (4a′), (5a), (5b′), (VI), (7a′), or a pharmaceutically acceptable salt or solvate thereof, 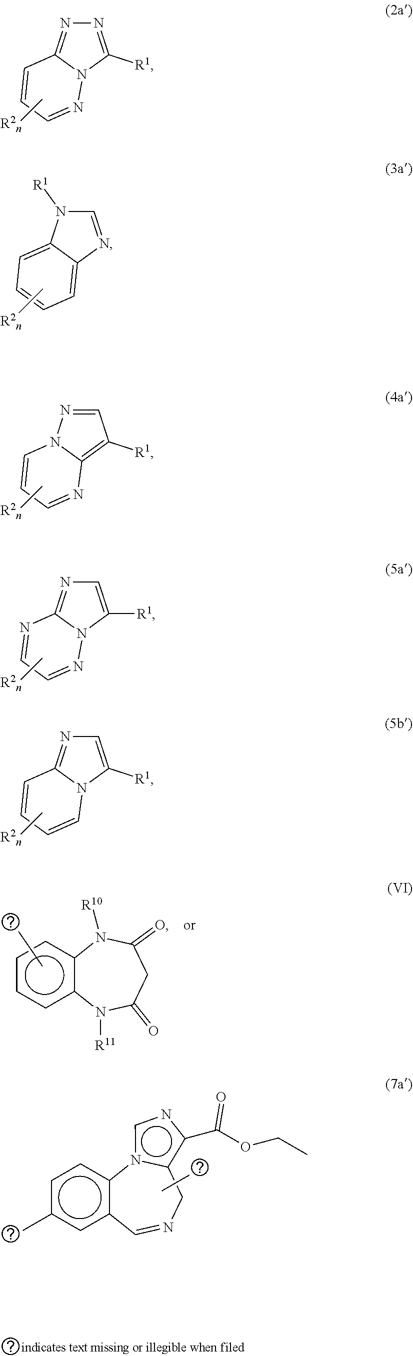
wherein
R₁₀ is a substituted or unsubstituted aryl, a substituted or unsubstituted C₁-C₃ alkyl or hydrogen,
R₁₁ is a substituted or unsubstituted aryl, a substituted or unsubstituted C₁-C₃ alkyl, or hydrogen, or
p of R¹²ₚ is 1 and R¹² is I, Br, Cl or F.

11. The method of claim 6, wherein the compound is of the general formula (2a″), (3a″), (4a″), (5a″), (5b″), (VIa″), (7a″), or a pharmaceutically acceptable salt or solvate thereof, 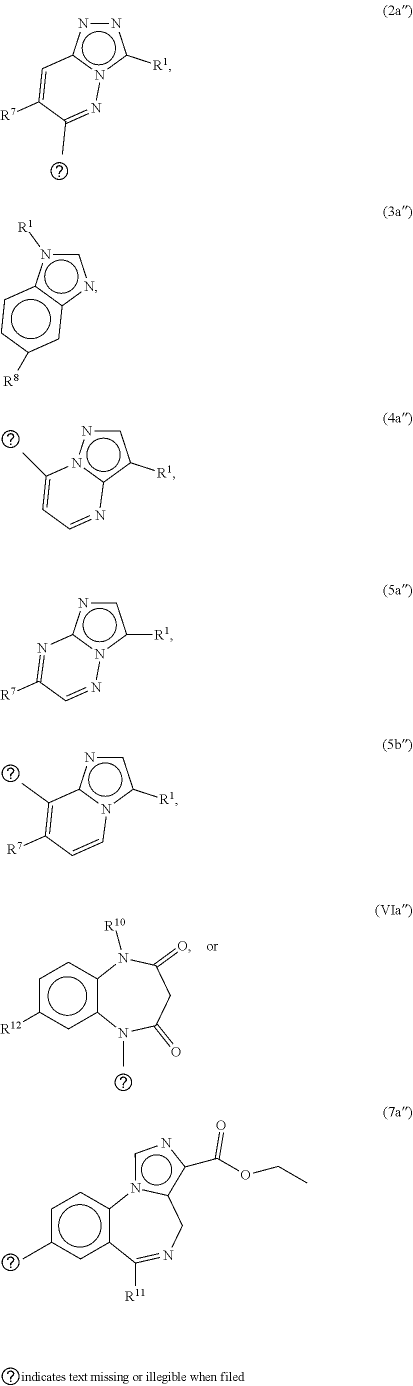
wherein R⁷ is:
an unsubstituted C₁-C₆ alkyl,
an unsubstituted C₃-C₈ cycloalkyl,
an unsubstituted C₁-C₆ alcohol,
R⁸ is:
—O—CH₂—R⁴, wherein R⁴ is a substituted or unsubstituted 5-membered heteroaryl, or an unsubstituted C₁-C₆ alcohol,
R⁹ is:
an unsubstituted C₆ heteroaryl, or
a halogen, or
R⁹ is an unsubstituted 6-membered heteroaryl in formula (4a″) or a halogen in formula (5b″),
R¹⁰ is a C₁-C₃ alkyl or hydrogen,
R¹¹ is a substituted or unsubstituted aryl or heteroaryl,
R¹⁴ is a substituted or unsubstituted aryl or heteroaryl,
R¹² is I, Cl, Br or F, or
R⁵ is C₂ alkinyl or I.

12. The method of claim 6, wherein the compound is of the general formula (8), or a pharmaceutically acceptable salt or solvate thereof: 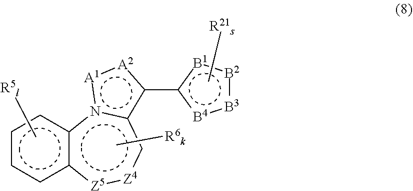
wherein
Z⁴ and Z⁵ are independently —C, —N, —S or —O,
A¹ and A² are independently —C, —N or —C(C═O)—O—R⁷, wherein R⁷ is alkyl,
B¹, B², B³, and B⁴ are independently —C, —N, or —O,
l of R⁵ₗ is 1 or 2, wherein R⁵ is C₁-C₄ alkinyl or a halogen,
k of R⁶ₖ is 1, 2, 3 or 4, wherein each R⁶ is independently a substituted or unsubstituted aryl, a substituted or unsubstituted heteroaryl, a substituted or unsubstituted C₁-C₃ alkyl, or hydrogen, and
s of R²¹ₛ, is 1, 2, 3 or 4, wherein each R²¹ is independently hydrogen or C₁-C₆ alkyl.

13. The method of claim 6, wherein the compound is of the general formula (5″), or a pharmaceutically acceptable salt or solvate thereof:

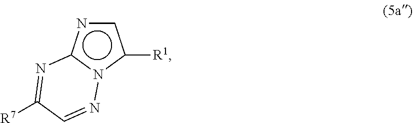

14. The method of claim 13, wherein R¹ is:
an unsubstituted biphenyl,
a substituted biphenyl comprising at least one —CN as a substituent, or
a substituted biphenyl comprising at least one F as a substituent.

15. The method of claim 14, wherein R¹ is a substituted biphenyl comprising —CN and F.

16. The method of claim 13, wherein R⁷ is an unsubstituted C₁-C₆ alcohol.

17. A method of treating fibromyalgia comprising administering to a subject in need thereof a therapeutically effective amount of a compound that is a GABA_{A} receptor modulator, wherein the compound is 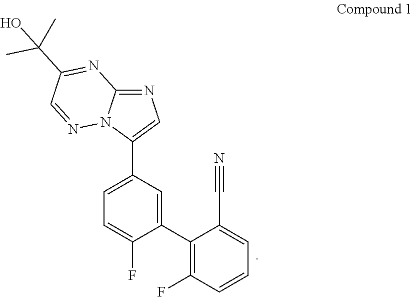 [SMILES: CC(C)(O)c1cnn2c(-c3ccc(F)c(-c4c(F)cccc4C#N)c3)cnc2n1]
or a pharmaceutically acceptable salt or solvate thereof.

18. The method of claim 1, wherein the subject is a human.

19. The method of claim 1, wherein the subject is a dog.

20. The method of claim 1, wherein the method comprises administering the compound, or a pharmaceutically acceptable salt or solvate thereof, at a dose of from about 0.001 mg/kg per day to about 3.0 mg/kg per day of a body weight of the subject.

21. A method of treating pain in a subject comprising administering to the subject an amount of a pharmaceutical composition comprising a compound selected from the following: 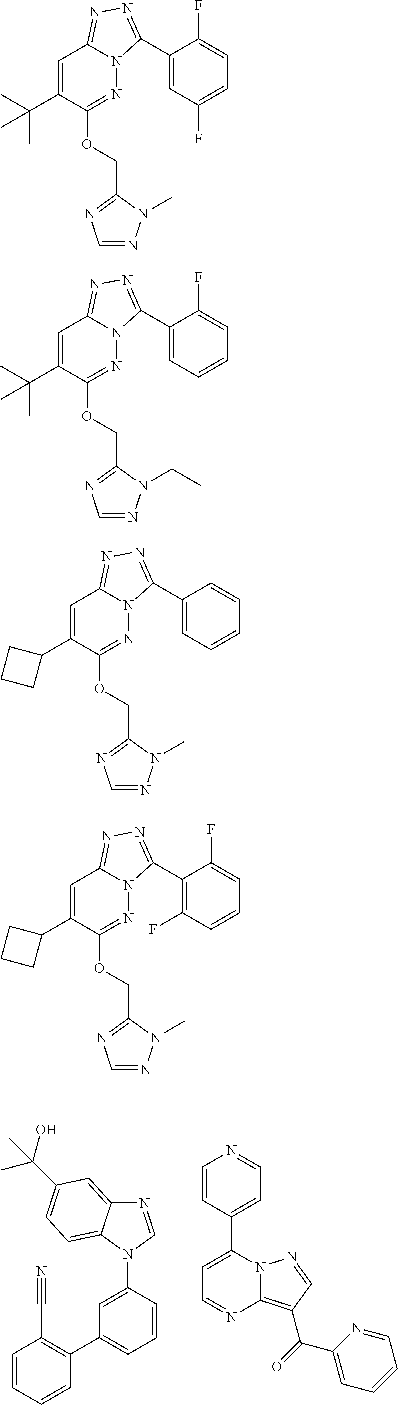 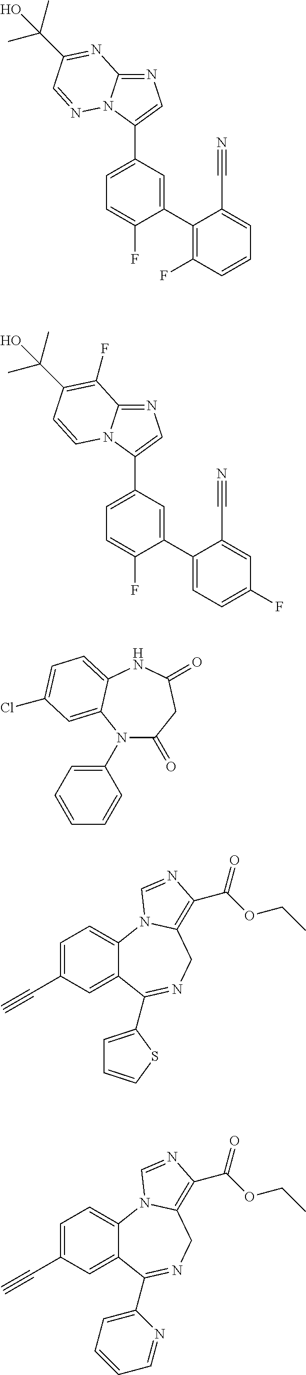 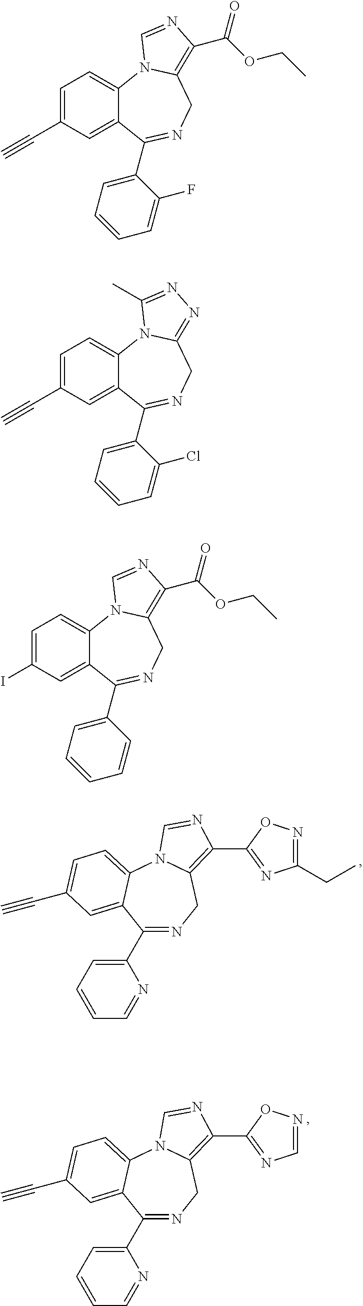 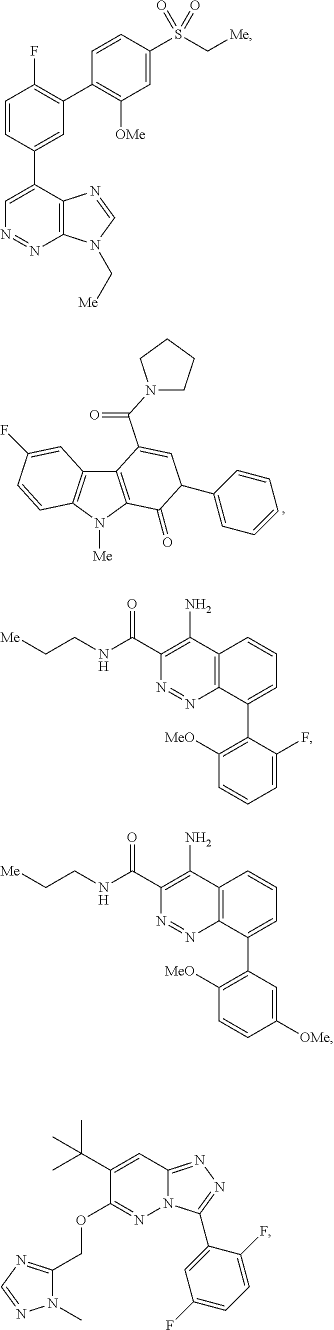 [SMILES: CCn1cnc2c(-c3ccc(F)c(-c4ccc(S(=O)(=O)CC)cc4OC)c3)cnnc21 ; Cn1c2c(c3cc(F)ccc31)C(C(=O)N1CCCC1)=CC(c1ccccc1)C2=O ; CCCNC(=O)c1nnc2c(-c3c(F)cccc3OC)cccc2c1N ; CCCNC(=O)c1nnc2c(-c3cc(OC)ccc3OC)cccc2c1N ; Cn1ncnc1COc1nn2c(-c3cc(F)ccc3F)nnc2cc1C(C)(C)C] 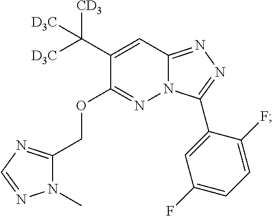 [SMILES: [2H]C([2H])([2H])C(c1cc2nnc(-c3cc(F)ccc3F)n2nc1OCc1ncnn1C)(C([2H])([2H])[2H])C([2H])([2H])[2H]]
and pharmaceutically acceptable salts or solvents thereof, wherein the amount is effective to treat the pain when the compound, or a pharmaceutically acceptable salt or solvate thereof, is administered at a dose of from about 0.001 mg/kg per day to about 3.0 mg/kg per day of a body weight of the subject.

22. The method of claim 21, wherein the compound is: 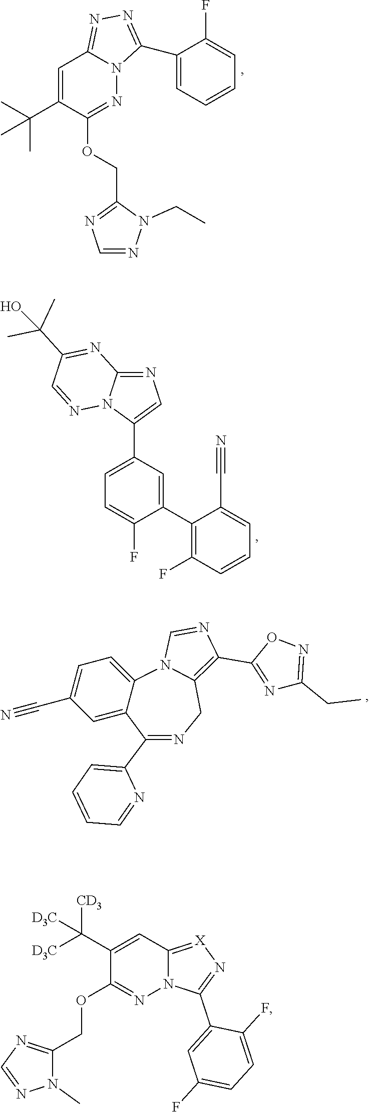 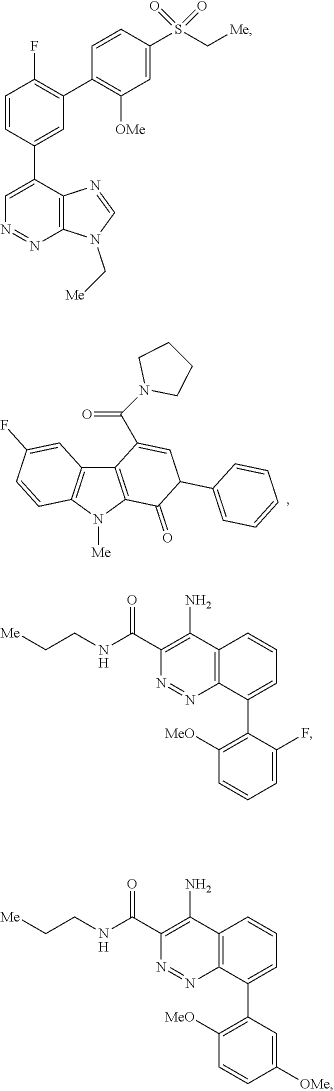
or a pharmaceutically acceptable solvate thereof.

23. The method of claim 21, wherein the compound is 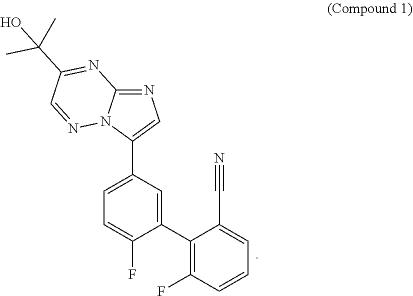 [SMILES: CC(C)(O)c1cnn2c(-c3ccc(F)c(-c4c(F)cccc4C#N)c3)cnc2n1]
or a pharmaceutically acceptable salt or solvate thereof.

24. The method of claim 21, wherein the amount is effective to treat the pain when the compound, or a pharmaceutically acceptable salt or solvate thereof, is administered at a dose of at most about 0.3 mg/kg per day of a body weight of the subject.

25. The method of claim 21, wherein the amount is effective to treat the pain when the compound, or a pharmaceutically acceptable salt or solvate thereof, is administered at a dose of at most about 0.03 mg/kg per day of a body weight of the subject.

26. The method of claim 21, wherein the pain is related to central nervous system sensitization.

27. The method of claim 21, wherein the pain is chronic.

28. The method of claim 21, wherein the subject is a human.

29. The method of claim 21, wherein the subject is a dog.

30. The method of claim 21, wherein the pharmaceutical composition further comprises a pharmaceutically acceptable excipient, diluent, or carrier.

31. The method of claim 21, wherein the pharmaceutical composition comprises a carrier, and wherein the carrier is methyl cellulose.

32. The method of claim 21, wherein the administering comprises an oral administration.

33. The method of claim 21, wherein the administering is performed at least once a day.

34. A method of treating fibromyalgia in a subject in need thereof comprising administering to the subject a pharmaceutical composition comprising a compound of: 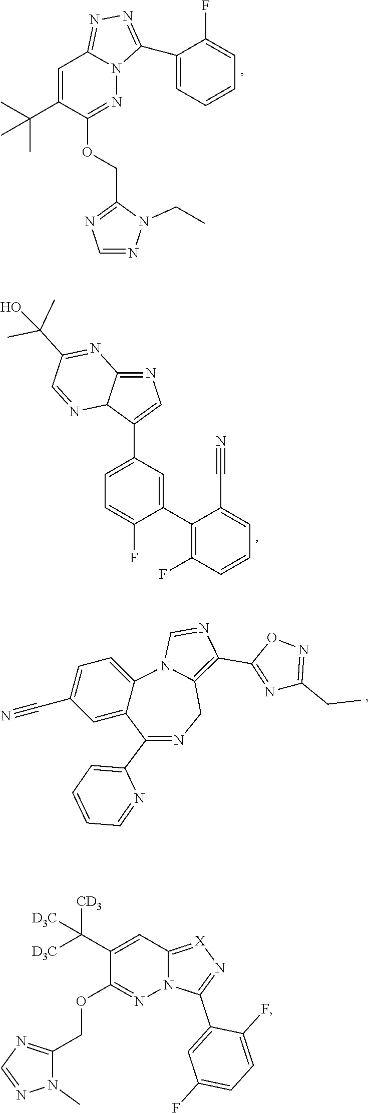 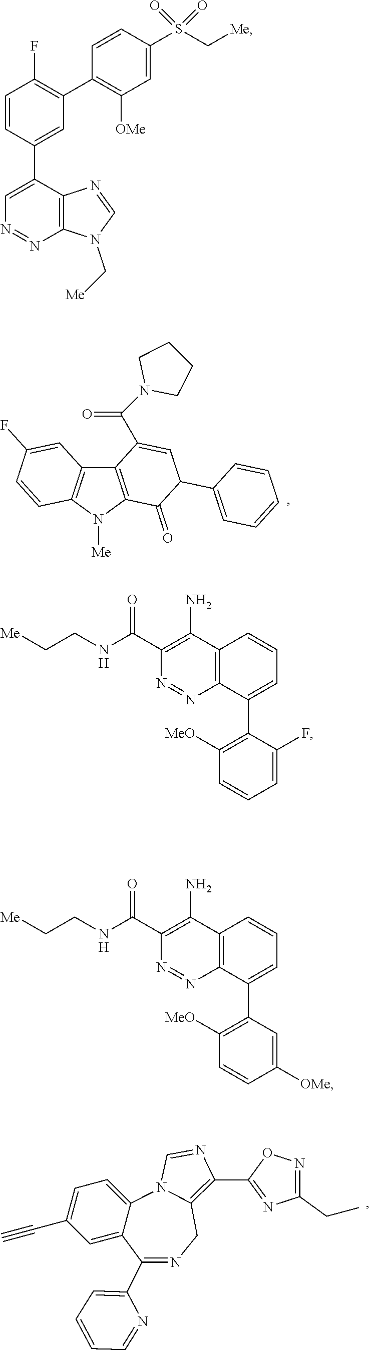
or a pharmaceutically acceptable salt or solvate thereof, and a pharmaceutically acceptable excipient, diluent, or carrier.

35. The method of claim 34, wherein the pharmaceutical composition comprises 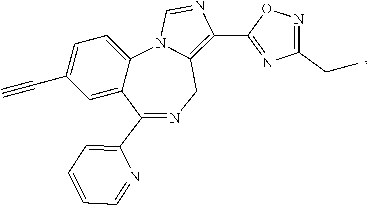 [SMILES: C#Cc1ccc2c(c1)C(c1ccccn1)=NCc1c(-c3nc(CC)no3)ncn1-2]
or a pharmaceutically acceptable salt or solvate thereof.

36. The method of claim 34, wherein the pharmaceutical composition comprises 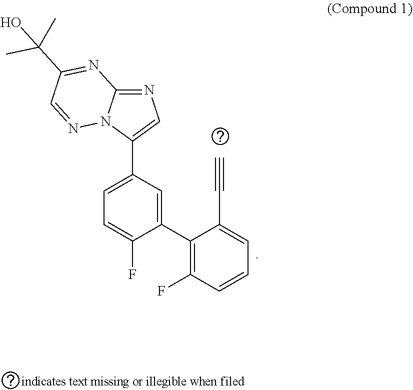 [SMILES: C#Cc1cccc(F)c1-c1cc(-c2cnc3nc(C(C)(C)O)cnn23)ccc1F]
or a pharmaceutically acceptable salt or solvate thereof.

37. The method of claim 34, wherein the compound or a pharmaceutically acceptable salt or solvate thereof is administered at a dose of from about 0.001 mg/kg per day to about 3.0 mg/kg per day of a body weight of the subject.

38. The method of claim 34, wherein the compound or a pharmaceutically acceptable salt or solvate thereof is administered at a dose of at most about 0.3 mg/kg per day of a body weight of the subject.

39. The method of claim 34, wherein the compound or a pharmaceutically acceptable salt or solvate thereof is administered at a dose of at most about 0.03 mg/kg per day of a body weight of the subject.

40. The method of claim 1, wherein the subject has a fibromyalgia-associated allodynia.
